(19) United States
(12) Patent Application Publication
(10) Pub. No.: US 2008/0311580 A1
(43) Pub. Date: Dec. 18, 2008

(54) Novel genes encoding proteins involved in proanthocyanidin synthesis

(75) Inventors: Sharon Abrahams, Cook (AU); Anthony Richard Ashton, Cook (AU); Gregory John Tanner, O'Connor (AU); Philip John Larkin, Weston (AU)

Correspondence Address: Cooper & Dunham LLP, 1185 Avenue of the Americas, New York, NY, 10036, US

(73) Assignee: Commonwealth Scientific and Industrial Research Organization

(21) Appl. No.: 12/011,673

(22) Filed: Jan. 28, 2008

Related U.S. Application Data
(63) Continuation of application No. 11/076,164, filed on Mar. 9, 2005, now abandoned.
(60) Provisional application No. 60/552,068, filed on Mar. 9, 2004.

(51) Int. Cl. C12Q 1/68

(52) U.S. Cl. 435/6

(57) ABSTRACT

This inventions provides an isolated protein or polypeptide having activity in the synthesis of proanthocyanidin (PA) polymer from epicatechin or catechin in plants, and which is not naturally regulated by the TT2 or TT8 regulators, or a fragment comprising at least about 10 contiguous amino acids derived from said protein or polypeptide, particularly TDS1, TDS2, TDS3, TDS4, TDS5, or TDS6 protein, or fragment thereof.

Also provided are nucleic acid encoding such proteins or polypeptides; plant cells and plants containing and expressing such nucleic acids; antibodies that bind to such proteins or polypeptides; and related methods.

### FIELD OF THE INVENTION

The present invention relates generally to isolated proteins or polypeptides which are involved in proanthocyanidin (PA) synthesis and vacuole development in plants, and nucleic acid molecules encoding same and their use in regulating the biosynthesis and accumulation of proanthocyanidins in plants. The isolated proteins or polypeptides and nucleic acid molecules of the present invention are useful for modifying the pasture quality of legumes, and, in particular, for producing bloat-safe forage crops, or crops having enhanced nutritional value, enhanced disease resistance or pest resistance, or enhanced malting qualities.

### GENERAL

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of said steps or features.

Throughout this specification, unless the context requires otherwise the word “comprise”, and variations such as “comprises” and “comprising”, will be understood to imply the inclusion of a stated integer or step or group of integers or steps but not the exclusion of any other integer or step or group of integers or steps. The present invention is not to be limited in scope by the specific embodiments described herein, which are intended for the purposes of exemplification only. Functionally-equivalent products, compositions and methods are clearly within the scope of the invention, as described herein.

Bibliographic details of the publications referred to by author in this specification are collected at the end of the description. Reference herein to prior art, including any one or more prior art documents, is not to be taken as an acknowledgment, or suggestion, that said prior art is common general knowledge in Australia or forms a part of the common general knowledge in Australia.

As used herein, the term “derived from” shall be taken to indicate that a particular integer or group of integers has originated from the species specified, but has not necessarily been obtained directly from the specified source.

This specification contains nucleotide sequence information prepared using the program Patentin Version 3.1, presented herein after the claims. Each nucleotide sequence is identified in the sequence listing by the numeric indicator <210> followed by the sequence identifier (e.g. <210>1, <210>2, etc). The length, type of sequence (DNA, protein (PRT), etc) and source organism for each nucleotide sequence are indicated by information provided in the numeric indicator fields <211>, <212> and <213>, respectively. Nucleotide sequences referred to in the specification are defined by the term “SEQ ID NO:”, followed by the sequence Identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymidine, Y represents a pyrimidine residue, R represents a purine residue, M represents Adenine or Cytosine, K represents Guanine or Thymidine, S represents Guanine or Cytosine, W represents Adenine or Thymidine, H represents a nucleotide other than Guanine, B represents a nucleotide other than Adenine, V represents a nucleotide other than Thymidine, D represents a nucleotide other than Cytosine and N represents any nucleotide residue.

### BACKGROUND TO THE INVENTION

Pasture bloat is a serious risk for cattle grazing on forage legumes. Bloat often results in loss of livestock, and productivity may also be reduced considerably by the stress of sub-lethal bloat. The fear of bloat and the required vigilance also has a negative impact on dairy farmers lifestyle.

Bloat is a major constraint on dairy farm profitability, and also impacts significantly on beef production. Because of high nutritive value, white clover and lucerne are used extensively in the dairy industry. Accordingly, there is a clear need in the dairy industry for the production of bloat-safe lucerne and white clover crops.

### DESCRIPTION OF THE PRIOR ART

It is known that bloat is caused by the production of a highly stable protein foam in the rumen during the initial rapid fermentation of fresh legume forage. There is negative correlation between the level of condensed tannins in the foliage of legumes and the ability of particular legumes to induce bloating in livestock animals such as cattle, which have been grazed thereon (Lyttleton, 1971; Li et al., 1996). Furthermore, Tanner et al. (1995) have demonstrated that the presence of foliar proanthocyanidin significantly reduces the compressive strength of protein foams formed from red clover leaf protein. There is also correlation between the presence of condensed tannins in forage crops such as Lotus comiculatus, Onobrychis viciifolia and Trifolium arvense, and the levels of post-rumen protein availability and protein loss in rumenants.

In general, there is a higher efficiency of protein utilization by rumenous livestock animals fed on forage crops which contain condensed tannins than by animals fed on crops with low tannin content (Terrill et al, 1992; McNabb et al, 1993; Wang et al, 1994; Lee et al, 1995; Niezen et al, 1995). Without tannins, the rapid release of soluble protein from the soft legume leaf cells results in more protein than can be incorporated into rumen microbial protein. The excess soluble protein is broken down to ammonia which is absorbed and excreted as urea. This represents a major wastage of dietary protein; approximately 30-40% of dietary protein may be lost due to rumen degradation (Barry and Reid, 1985).

Flavonoids are a diverse group of secondary metabolites that includes the monomeric flavanols and anthocyanins, as well as the polymeric proanthocyanidins (PA) or condensed tannins. The anthocyanin and PA biosynthetic pathways in Arabidopsis share common intermediates to cyanidin, which can be diverted to PA synthesis via anthocyanidin reductase, also known as BANYULS (BAN), or to anthocyanin synthesis by UDP-glucose flavonoid 3-O-glucosyl transferase (UFGT). In other plants an alternate branch point exists at 2,3-trans-3,4-c/s-leucocyanidin, which can be used by the enzyme leucoanthocyanidin reductase to make catechin (Tanner and Kristiansen, 1993). In Arabidopsis, although anthocyanins and flavonols are widespread in the plant, PA synthesis occurs only in a single endothelial cell layer in the developing seed coat. The Arabidopsis transparent testa (tt) mutants, which have a pale seed phenotype, define many of the common biochemical steps in the formation of anthocyanin and PA, such as chalcone synthase, chalcone isomerase (CHI), flavanone 3-hydroxylase, flavanone 3′-hydroxylase and DFR (Feinbaum and Ausubel, 1988; Schoenbohm et al., 2000; Shirley et al., 1992). However, it has become clear that the tt mutants define genes involved not only in anthocyanin synthesis, but also those specifically involved in PA biosynthesis. For example. TT genes such as TT12 and TT2 appear to be involved in PA biosynthesis or the regulation of genes involved in the early steps of PA biosynthesis, rather than anthocyanin synthesis (Debeaujon et al., 2001; Nest et al., 2001). Indeed, we have recently shown that the TANNIN DEFICIENT SEEM (TDS4) gene encodes leucoanthocyanidin reductase (LDOX), and that in Arabidopsis, LDOX is involved not only in anthocyanin synthesis but also PA synthesis (Abrahams et al., 2003). This finding suggests the need for a re-evaluation of the distinction between the anthocyanin and PA biosynthetic pathways.

### SUMMARY OF THE INVENTION

In work leading up to the present invention, the inventors have isolated nucleotide sequences encoding proteins or polypeptides which are involved in PA synthesis in plants, in particular nucleotide sequences encoding proteins or polypeptides involved in assembly of PA polymer from epicatechin or catechin.

Accordingly, in one aspect the present invention provides an isolated protein or polypeptide having activity in the synthesis of proanthocyanidin (PA) polymer from epicatechin or catechin in plants, and which is not naturally regulated by the TT2 or TT8 regulators, or a fragment comprising at least about 10 contiguous amino acids derived from said protein or polypeptide.

The isolated proteins or polypeptides of the present invention are members of the TDS (Tannin Deficient Seed) family of proteins, encoded by the tds genes identified by screening mutants in the PA pathway in Arabidopsis (Abrahams et al, 2002), but do not include the TDS4 protein encoded by the tds4 gene which has been identified as leucoanthocyanidin dioxygenase (LDOX) (Abrahams et al, 2003) and which is active in an earlier part of the PA synthesis pathway, specifically in the synthesis of the monomer epicatechin, or the TT12 protein which is a MATE transporter implicated in transport of PA intermediates into the vacuole (Debeaujon et al., 2001). These proteins or polypeptides are referred to herein, for convenience, as “TDS proteins”.

Specifically, in this aspect the present invention relates to an isolated protein or peptide selected from the group consisting of the TDS1, TDS2, TDS3, TDS5 and TDS6 proteins, or a fragment thereof.

Preferably, the isolated protein or peptide is the TDS6 or TDS2 protein hereinafter described in detail, or a fragment thereof. As disclosed herein, the TDS6 protein is a chalcone isomerase (CHI)-like protein that acts in the synthesis of PA from epicatechin or catechin, while the TDS2 protein, which includes a single C2 domain, is involved in controlling the release of PA related intermediates from vesicles into the vacuole. The protein may be an enzyme such as an isomerase, epimerase or a PA condensing (polymerising) enzyme.

In another aspect, the present invention provides an isolated protein or polypeptide which comprises (i) an amino acid sequence selected from the group consisting of SEQ ID NO: 2 and SEQ ID NO: 4, or an orthologue or homologue thereof; (ii) an amino acid sequence having at least 40% identity overall to an amino acid sequence of (i) above; or (iii) a fragment comprising at least about 10 contiguous amino acids derived from (i) or (ii).

The present invention also provides an isolated nucleic acid molecule comprising a nucleotide sequence selected from the group consisting of: (1) a nucleotide sequence that encodes a protein or polypeptide having activity in the synthesis of proanthocyanidin (PA) polymer from epicatechin or catechin in plants, and which is not naturally regulated by the TT2 or TT8 regulators, (ii) a nucleotide sequence that encodes a fragment comprising at least about 10 contiguous amino acids derived from said protein or polypeptide; and (iii) a nucleotide sequence that is complementary to (i) or (ii). The isolated nucleic acid molecule comprises DNA and/or RNA.

In this aspect, the present invention relates to an isolated nucleic acid molecule that encodes a protein or polypeptide selected from the group consisting of the TDS1, TDS2, TDS3, TDS5 and TDS6 protein, or a fragment thereof. These nucleic acid molecules are referred to herein, for convenience, as “tds nucleic acid molecules”. More particularly, the isolated nucleic acid molecule encodes the TDS6 or TDS2 proteins described herein in detail, or a fragment thereof.

In another aspect, the present invention extends to an isolated nucleic acid molecule comprising a nucleotide sequence selected from the group consisting of:
(i) a nucleotide sequence having at least about 40% identity overall to SEQ ID NO: 1 or SEQ ID NO: 3, or to a coding region thereof;
(ii) a nucleotide sequence that encodes a protein or polypeptide having at least about 40% identity overall to SEQ ID NO: 2 or SEQ ID NO: 4;
(iii) a nucleotide sequence that encodes a fragment comprising at least about 10 contiguous amino acids derived from the protein or polypeptide of (ii);
(iv) a nucleotide sequence that hybridises under at least low stringency conditions to at least about contiguous nucleotides of any one of (i) to (iii); and
(v) a nucleotide sequence that is complementary to any one of (i) to (iv).

This invention clearly extends to any gene constructs that comprise the isolated nucleic acid molecule of the present invention, such as, for example, any expression gene constructs produced for expressing said nucleic acid molecule in a bacterial, insect, yeast, plant, fungal, or animal cell. Accordingly, a further aspect of the present invention is directed to a gene construct comprising an isolated nucleic acid molecule as described above. The gene construct preferably comprises the isolated nucleic acid molecule operably linked to a heterologous promoter which is capable of expression in a plant cell.

A further aspect of the invention contemplates an isolated cell such as a plant cell comprising a non-endogenous tds nucleic acid molecule or gene construct as described above, preferably wherein said tds nucleic acid molecule is present in said cell in an expressible format.

A further aspect of the invention contemplates a transformed plant comprising a non-endogenous tds nucleic acid molecule as described above introduced into its genome, in an expressible format. Preferably, the transformed plant of the invention further expresses a non-endogenous TDS protein encoded by the nucleic acid molecule in at least some cells or tissues. This aspect of the invention clearly extends to any plant parts, or progeny plants, that are derived from the primary transformed plant.

A still further aspect of the invention contemplates a method of enhancing the expression of a TDS protein in a plant or plant tissues comprising introducing to the genome of said plant a non-endogenous tds nucleic acid molecule in an plant-expressible format.

A still further aspect of the invention contemplates a method of reducing the expression of a TDS protein in a plant or plant tissues comprising introducing to the genome of said plant a molecule selected from the group consisting of: an antisense molecule, a PTGS molecule, and a co-suppression molecule, wherein said molecule comprises at least about 20 contiguous nucleotides of a tds nucleic acid molecule or complementary to a tds nucleic acid molecule, in an plant-expressible format A still further aspect of the invention contemplates a method of reducing the expression of a TDS protein in a plant or plant tissues comprising introducing to the genome of said plant a ribozyme molecule, wherein said molecule comprises at least two hybridising regions each of at least 5 contiguous nucleotides complementary to a tds nucleic acid molecule, separated by a catalytic domain capable of cleaving an RNA encoding a TDS protein of the invention, in an plant-expressible format.

The present invention further extends to the use of the transformed plants and methods described herein to reduce the severity or incidence of bloat in pasture animals.

### BRIEF DESCRIPTION OF THE DRAWINGS

FIG. 1. The first part of the anthocyanin and proanthcyanidin (PA) synthesis pathway in plants and the structure of PA-related intermediates is shown, from chalcone synthase (CHS) to flavonol synthase (FLS). The difference between 2,3-c/s and 2,3-trans isomers of flavan-3-ols is shown, using catechin and epicatechin as examples (inset). Other abbreviations: CHI, chalcone isomerase; F3H, flavanone 3D-hydroxylase; F3′H, flavonoid 3′ hydroxylase.

FIG. 2. The second part of the anthocyanin and PA synthesis pathway in plants and the structure of PA-related Intermediates is shown, from dihydroquercetin to PA. The branch between anthocyanin and PA biosynthesis occurs at cyanidin. Abbreviations: DFR, dihydroflavonol reductase; LAR, leucoanthocyanidin reductase; LDOX, leucoanthocyanidin dioxygenase, BAN, anthocyanidin reductase; UFGT, UDPG glucose-flavonoid 3-O-glucosyl transferase. The enzymatic steps altered in the transparent testa (TT) mutants referred to in the text are shown, the regulatory gene affected is indicated in parentheses. The positions of the genetic mutations in the TDS mutants are indicated.

FIG. 3. Structure of the flavan-3-ols (I and II) and isomers of the flavan-3,4-diola (III to VI). The position of the A, B and C rings, and the isomerisation of the hydroxyl at the 3 position on the C ring is shown. For compound I: when R, R1=H the compound is 2,3-trans-afzelechin; when R=OH, R1=H: 2,3-trans-catechin; when R, R1=OH: 2,3-trans-gallocatechin. For compound II, when R, R1=H: 2,3-cis-epiafzelechin; when R=OH, R1=H: 2,3-cis-epicatechin; when R, R1=OH: 2,3-cis-epigallocatechin. Compound III: 2,3-trans-3,4-cis-leucocyanidin. Compound IV: 2,3-trans-3,4-trans-leucocyanidin. Compound V: 2,3-cis-3,4-trans-epileucocyanidin. Compound VI: 2,3-cis-3,4-cis-epileucocyanidin. The extension unit identified in Arabidopsis PA is represented by 2,3-c/s-3,4-trans-epi-leucocyanidin. The flavan-3,4-diol often shown in pathways is 2,3-trans-3,4-c/s-leucocyanidin.

FIG. 4. Wild type and mutant mature seed stained with DMACA. A to I, pools of mature seed including Ws-2, tds1, tds2, tds3-1, tds4, tds5, tds6, U8-4 and U7-3 showing differences in staining with DMACA. J and K, enlarged images comparing Ws-2, tds4 and H7-3 (J) and Ws-2, ban, tt4 and tds2 (K). The bar represents 0.05 mm (A to I) and 0.025 mm (J, K).

FIG. 5. Quantitation of anthocyanin and PA. A, quantitation of leaf anthocyanin as a % of wild type values, measured in duplicate, for different genotypes as indicated under each bar. B, mature seed anthocyanin shown as a % of wild type, measured in duplicate. C, PA measured in mature seed for Ws-2 tds4, Col-7, tt7-3 and tds6, measured in duplicate. Results shown as a % relative to Ws-2 wild type. Error bars represent standard deviation.

FIG. 6. Nucleotide and encoded amino acid sequences of TDS6 cDNA from Arabidopsis.

FIG. 7. TDS6 gene structure and the T-DNA insertion sites in the TDS6 gene that create the tds6 mutants. The diagram shows the intron/exon arrangement of the TDS6 gene and the positions of the T-DNA insertions in tds6-1 and tds6-2. The positions of primer sites are indicated by small triangles.

FIG. 8. Mature Arabidopsis seeds unstained (A, C, E) or stained (B, D, F, G, H) with DMACA. Wild-type (A and B), tds6-1 (C and D), tds6-2 (E and F), or Ws6-1 transformed with 35S-TDS6 line 2 (G) and 35S-TDS6 line 3 (H).

FIG. 9. Quantitation of epicatechin monomer and PA (polymer) of complemented tds6-1 transgenics.

FIG. 10. TDS6 is a very late PA biosynthetic gene that is not regulated by TT2 or TT8. RT-PCR analysis of TDS6 and 7712 mRNA expression in Arabidopsis tissues. L, leaf; St, stem; Fb, flower buds; 1-6, siliques from two terminal cell stage until walking stick stage of development; H2A, histone H2A

FIG. 11. RT-PCR analysis of HISTONE, CHS, TT12, DFR and 7DS6 mRNA expression in developing siliques from Col7, tt2 and tt8 plants.

FIG. 12. Sequence alignment of TDS6 and CHI proteins produced by ClustalW. Fully conserved amino acids are represented by an asterisk (*), and conservation of strong groups indicated by a colon (:) in the consensus line. Dashes have been introduced to maintain homology. Abbreviations: Osa, Oryza sativa (AAM13448); Hvu, Hordeum vulgare (AAM13449); Zma, Zea mays (Q08704); Ath, Arabidopsis thaliana (CAB94981); Csl, Citrus sinensis (BAA36552); Vvi, Vitus vinifera (P51117); Eum, Elaeagnus umbellate (065333); Sme, Saussurea medusa (AAM48130); Phy, Pelunia hybrids (P11650); Gma, Glycine max (AAK69432); Pvu, Phasaolus vulgaris (P14298); Msa1, Medicago sativa (P28012); TDS6, A. thaliana (NP_{—}568154).

FIG. 13. ClustalW alignment of TDS6 and CHI proteins represented as a tree.

FIG. 14. Quantitation of epicatechin and PA in maturing siliques and expression of the TDS6 gene

A. Graphical representation of epicatechin and PA amounts extracted from maturing Arabidopsis siliques.
B. TLC showing the difference between epicatechin and PA fractions isolated from developing siliques. The volume loaded was normalised on dry weight measurements. The upper TLC shows the ethyl acetate fraction of 70% acetone extracts 1 to 10, containing mostly epicatechin monomer and some dimer, corresponding to samples 1 to 10 shown in A. The lower TLC shows the range of PA polymers remaining in the aqueous phase, after the removal of acetone and ethyl acetate extraction. PA, Onobrychis PA; ec, epicatechin; d, catechin monomer; c2, catechin dimer; c3, catechin trimer.
C. RT-PCR analysis of RNA extracted from samples 1 to 10, showing the expression patterns of TDS6 and histone H2A.

FIG. 15. Nucleotide and encoded amino acid sequences of TDS2 cDNA from Arabidopsis.

FIG. 16. TDS2 gene arrangement and TDS2 protein structure and sequence compared to other C2 domain containing proteins. A. The T-DNA insertion site in the TDS2 gene that created the tds2-1 mutation. B. The relative position of the single C2 domain (rectangle) in the TDS2 protein is shown schematically. C. Sequence comparison between the TDS2 and PKC C2 domains. Gaps (dashes) have been introduced to maximise similarity. The A (DPYW), B (KLTK) and C (VNPEWNEDLTL) subdomains are shaded. Amino acid differences at positions 22, 23, 33, 46, 48, 49, 54, 56, 60, 78, 81, 91, 95, and 102 with respect to the TDS2 amino acid sequence are conservative.

FIG. 17. A comparison of C2 domains and their relative positions in a number of protein families.

FIG. 18. Mature Arabidopsis seeds unstained (left panels) or stained (right panels) with DMACA. Wild-type Ws-2 (A and B), tds2 (C and D), 35S:TDS2-2 (E and F), 35S:TDS2-9 (G and H) and 35S:TDS2-10 (I and J).

FIG. 19. Quatitation of epicatechin and PA polymers extracted from mature seed of Ws-2, tds2 and lines transformed with 35S:TDS construct.

FIG. 20. TDS2 expression. A. RT-PCR plus Southern blot analysis of TDS2 and TT12 mRNA expression in Arabidopsis tissues. L, leaf; St, stem; F, flowers; 1-6, siliques from two-terminal cell stage until walking stick stage of embryo development; H2A, histone H2A. B. RT-PCR plus Southern blot analysis of the expression of TDS2 CHS, TT12 and DFR in wild-type, tt2 and H8 mutant silique material. H2A, histone H2A.

FIG. 21. Localisation of PA-related intermediates in Ws-2 and tds2 developing siliques. Light microscopy of developing Arabidopsis seeds stained with DMACA (A and B) or treated with osmium tetroxide and sectioned (C and D). v, vacuole; em, embryo; sc, seed coal.

FIG. 22. Endothelial cells of the tds2 mutant have numerous small vesicles located at the tonoplast. Osmium tetroxide treated EM sections of Ws-2 (A) and tds2 (B and C) developing seeds at the torpedo stage of development v, vacuole; cw, cell wall; c, cytoplasm.

### DETAILED DESCRIPTION OF THE INVENTION

One aspect of the present invention provides an isolated TDS protein or polypeptide having activity in the synthesis of proanthocyanidin (PA) polymer from epicatechin or catechin in plants, and which is not naturally regulated by the TT2 or TT8 regulators, or a fragment comprising at least about 10 contiguous amino acids derived from said protein or polypeptide. The isolated TDS protein or polypeptide does not include the TDS4 protein encoded by the tds4 gene which has been Identified as leucoanthocyanidin dioxygenase (LDOX) (Abrahams et al, 2003) and which is active in an earlier part of the PA synthesis pathway, specifically in the synthesis of the monomer epicatechin, or the TT12 protein which is a MATE transporter implicated in transport of PA intermediates into the vacuole (Debeaujon et al., 2001). The isolated TDS protein or polypeptide preferably is an enzyme selected from the group consisting of isomerase, epimerase and PA condensing enzyme. The substrates for such enzymes may be flavanoids such as flavanols or flavan-diols. The enzymes may catalyse the conversion of a leucocyanidin to the extension units used as monomers in PA synthesis, flavan-3,4-diols. It is preferred that fragments of the TDS protein or polypeptide have the same enzyme activity or biological activity as the full-length proteins disclosed herein.

Preferably, the isolated TDS protein is the TDS6 or TDS2 protein, or a fragment thereof.

The TDS 6 protein of the present invention includes four conserved sequences motifs. Consensus sequences for these four motifs are:

| | |
|---|---|
| Motif 1: | [KN] [PALT] L [SACHP] L [LMV] [GA] |
| | [QHNSTY] G [ILV] TD [IMV] E IHF [LI] [QH] |
| | [VI] K [FLY] [TNY] [AS] [IV] BVY [LI] |
| | [DEH] [PKST] |
| Motif 2: | [VI] VV [IA] KE [IL] KG [AS] QYGVQLE |
| Motif 3: | [VI] RDR [LV] [ASV] [AE] [EADIV] D [KL] |
| | [YF]] [ED] [ED] [ED] EE [TE] [EAST] LEK |
| | [VIL] [VAS] [GDE] FFQ [SAG] KYF [KR] |
| Motif 4: | ENANVV |

Most likely sequence for these four motifs of the TDS 6 protein are:

| | |
|---|---|
| Motif 1: | KPLSLLGQGITDIEIHFLQVKFTAIGVYLDP |
| Motif 2: | RVVVIKEIKGAQYGVQLE |
| Motif 3: | VRDRLAEEDKYEEEEETELEKVVGFFQSKYFK |
| Motif 4: | ENANVV. |

(Motif 1 corresponds to amino acids 21-51 in Arabidopsis: Motif 2 corresponds to amino acids 89-106 in Arabidopsis; Motif 3 corresponds to amino acids 109-140 in Arabidopsis: Motif 4 corresponds to amino acids 173-178 in Arabidopsis).

In a particularly preferred embodiment of the invention, the isolated TDS protein comprises the amino acid sequence set forth in SEQ ID NO: 2 or SEQ ID NO: 4.

Preferably, the isolated protein is substantially free of conspecific proteins.

Fragments of the isolated TDS protein of the present invention are useful for the purposes of producing antibodies against one or more B-cell or T-cell epitopes of the protein, which antibodies may be used, for example, to identify cDNA clones encoding homologues of the exemplified cDNA clones provided herein, or in immunohistochemical staining to determine the site of expression of the TDS protein. Those skilled in the art will appreciate that longer fragments than those consisting of only 10 amino acids in length may have improved utility than shorter fragments. Preferably, a fragment of a TDS protein of the invention will comprise at least about 20 contiguous amino acid residues, and more preferably at least about 50 contiguous amino acid residues derived from the native protein. Fragments derived from the internal region, the N-terminal region, or the C-terminal region of the native enzyme are encompassed by the present invention.

Fragments and isolated TDS proteins contemplated herein include modified peptides in which ligands are attached to one or more of the amino acid residues contained therein, such as a hapten; a carbohydrate; an amino acid, such as, for example, lysine; a peptide or polypeptide, such as, for example, keyhole limpet haemocyanin (KLH), ovalbumin, or phytohaemagglutinin (PHA); or a reporter molecule, such as, for example, a radionuclide, fluorescent compound, or antibody molecule. Glycosylated, fluorescent, acylated or alkylated forms of the subject peptides are particularly contemplated by the present invention. Additionally, homopolymers or heteropolymers comprising two or more copies of the subject TDS protein are contemplated herein. Procedures for derivatizing peptides are well-known in the art.

Notwithstanding that the present inventors have exemplified the TDS proteins of the invention from Arabadopsis, the invention clearly extends to isolated TDS proteins from other plant species, and, in the case of isolated proteins prepared by recombinant means, from any cellular source that supports the production of a recombinant TDS protein. Accordingly, the present invention clearly encompasses orthologues and homologues of the TDS proteins and fragments described herein.

In the present context, “homologues” of the TDS protein of the present invention refer to those proteins having a similar sequence to the TDS protein, while “orthologues” of the TDS protein are functionally equivalent homologues, that is homologues which have a similar activity to the TDS protein, notwithstanding any amino acid substitutions, additions or deletions thereto. An orthologue or homologue of the TDS proteins exemplified herein may be isolated or derived from the same or another plant species.

For example, the amino acids of a TDS protein may be replaced by other amino acids having similar properties, for example hydrophobicity, hydrophilicity, hydrophobic moment, charge or antigenicity, and so on. Substitutions encompass amino acid alterations in which an amino acid is replaced with a different naturally-occurring or a non-conventional amino acid residue.

Conservative amino acid substitutions are particularly contemplated herein for the production of orthologues or homologues of the TDS protein, such as, for example Gly [symbol] Ala; Ser [symbol] Thr, Met [symbol] Val [symbol] Ile [symbol] Leu; Asp [symbol] Glu; Lys [symbol] Arg; Asn [symbol] Gln; or Phe [symbol] Trp [symbol] Tyr. Such conservative substitutions will not generally inactivate the activity of the TDS protein.

The non-conservative substitution of one or more amino acid residues in the native TDS protein for any other naturally-occurring amino acid, or for a non-naturally occurring amino acid analogue, is also contemplated herein. Such substitutions generally involve modifications to charge, in particular charge reversals, or changes to the hydrophobicity of the TDS protein, and, more preferably, will modify the activity of the protein.

Amino acid substitutions are typically of single residues, but may be of multiple residues, either clustered or dispersed.

Orthologues and homologues of the isolated TDS proteins, wherein amino acid resides are deleted, or alternatively, additional amino acid residues are inserted are also contemplated herein. Amino acid deletions will usually be of the order of about 1-10 amino acid residues, and may occur throughout the length of the polypeptide. Insertions may be of any length, and may be made to the N-terminus, the C-terminus or be internal. Generally, insertions within the amino acid sequence will be smaller than amino- or carboxyl-terminal fusions and of the order of 1-4 amino acid residues. It is preferred that deletions or substitutions in TDS6 are in the regions of the protein outside of the conserved motifs described above.

The TDS protein of the present invention may comprise an amino acid sequence having at least about 40% identity overall to an amino acid sequence selected from the group consisting of: SEQ ID NO: 2 and SEQ ID NO: 4.

Preferably, the percentage identity overall to an amino acid sequence presented herein is at least about 50%, more preferably at least about 60%, even more preferably at least about 70%, even more preferably at least about 80%, even more preferably at least about 90%, and even more preferably at least about 95% or 99%. Those skilled in the art will be aware that the particular percentage identity between two or more amino acid sequences in a pairwise or multiple alignment may vary depending on the occurrence, and length, of any gaps in the alignment. Preferably, for the purposes of defining the percentage identity to the amino acid sequences listed herein, reference to a percentage identity between two or more amino acid sequences shall be taken to refer to the number of identical residues between said sequences as determined using any standard algorithm known to those skilled in the art that maximizes the number of identical residues and minimizes the number and/or length of sequence gaps in the alignment. For example, amino acid sequence identities or similarities may be calculated using the GAP programme and/or aligned using the PILEUP programme of the Computer Genetics Group, Inc., University Research Park, Madison, Wis., United States of America. Alternatively or in addition, wherein more than two amino acid sequences are being compared, the ClustalW programme of Thompson et al (1994) can be used.

Those skilled in the art will be aware that the percentage identity to a particular sequence is related to the phylogenetic distance between the species from which the sequences are derived, and as a consequence, those sequences from species distantly-related to Arabidopsis are likely to have functionally-equivalent TDS proteins, albeit having a low percentage identity to SEQ ID NO: 2 or SEQ ID NO: 4 at the amino acid sequence level. Such distantly-related TDS proteins may be isolated without undue experimentation using the isolation procedures described herein, and as a consequence, are clearly encompassed by the present invention.

Preferred sources of the TDS proteins of the present invention include any plant species known to produce tannins, and more particularly, catechin or epicatechin, in the seed coat, testa, pericarp, leaf, floral organ, or root. For example, preferred sources include those fodder or forage legumes, companion plants, food crops, trees, shrubs, or ornamentals selected from the group consisting of; Acacia spp., Acer spp., Actinidia spp., Aesculus spp., Agathis spp., Albizia spp., Alsophila spp., Andropogon spp., Arachis spp, Areca spp., Astelia spp., Astragalus spp., Baikiaea spp., Betula spp., Bruguiera spp., Burkea spp., Butea spp., Cadaba spp., Calliandra spp, Camellia spp., Canna spp., Cassia spp., Centroema spp, Chaenomeles spp., Cinnamomum spp., Coffea spp., Colophospermum spp., Coronillla spp., Cotoneaster spp., Crataegus spp., Cupressus spp., Cyathea spp., Cyclonia spp., Cryptomeria spp., Cymbopogon spp., Cynthea dealbata, Cyclonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia dura, spp., Eleusine coracana, Eragrestis spp., Erythrina spp, Eucalyptus robusta, Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Gilricldia spp, Gossypium hirsutum, Grevillea spp., Guibourtia coleosperma, Hedysarum spp., Hemarthia altissima, Heteropogon contortus, Hordeum vulgare, Hyparmenia rufa, Hypericum erectum, Hyperthelia dissoluta, Indigo incarnata, Iris spp., Leptarrhena pyrolifolia, Lespediza spp., Leucaena leucocephala, Loudetia simplex, Lotonus bainesli, Lotus spp., Macrotyloma axillara, Malus spp., Manihot esculenta, Medicego sativa, Metasequoia glyptostroboides, Musa sapientum, Onobrychls spp., Ornithopus spp., Peltophorum africanum, Persea gratissima, Phaseolus atropurpureus, Phoenix canadensis, Phormium cookianum, Photinia spp., Plcea glauca, Pinus spp., Podocaipus totara, Pogonarthria spp., Populus×euramericana, Prosopis cineraria, Pseudotsuga menziesii, Pterolobium stellatum, Pyrus communis, Quercus spp., Rhaphiotepsis umbellate, Rhopalostylis sapida, Rhus natalensis, Ribes spp., Robinia pseudoacacia, Rosa centifolla, Rubus spp., Salix spp., Schyzachyrium sanguineum, Sdadopitys verticillata, Sequoia sempervirens, Sequoiadendron giganteum, Sorghum bicobr, Sporobolus fimbrlatus, Stiburus alopecuroides, Stylosanthos humills, Tadehagi spp, Taxodium distichum, Themeda triandra, Trifolium spp., Triticum spp., Tsuga heterophylla, Vaccinium spp., Vicia sativa, Vitis vinifera, Watsonia pyramidata, and Zantedeschia aethioplca.

Even more preferably, the TDS protein of the invention is derived from a plant selected from the group consisting of: D. uncinatum, Medicago sativa, Medicago truncatula, Trifolium repens, Lotus corniculatus, Lotus japonicus, Nicotiana tabacum, Vitis vinifera, Camellia sinensis, Hordeum vulgare, Sorghum bicolor, Populus trichocarpa, Forsythia×intermedia, Thuja plicata, Pinus radiata, Pseudotsuga menziesii, and A. thaliana.

The seeds of any plant, or a tissue, cell or organ culture of any plant, are also preferred sources of the TDS protein.

The teaching provided herein clearly enables those skilled in the art to isolate a TDS protein of plants without undue experimentation. For example, the amino acid sequence of a Arabidopsis TDS protein, or the amino acid sequence of a fragment thereof, can be used to design antibodies for use in the affinity purification of immunologically cross-reactive proteins from other plants. Those skilled in the art will recognize that such immunologically cross-reactive proteins are likely to be TDS proteins, particularly if peptide fragments having amino acid sequences that are not highly-conserved between TDS and other proteins are used as immunogens to elicit the production of those antibodies. Alternatively, such antibodies can be used to isolate cDNA clones that express immunologically cross-reactive proteins according to any art-recognized protocol, such as, for example, the procedure disclosed by Huynh et al. (1985), and the expressed protein subsequently isolated or purified. The isolation or purification of the expressed protein is facilitated by expressing the TDS protein as a fusion protein with a tag, such as, for example, glutathione-S-transferase, FLAG, or oligo-Histidine motifs. Alternatively, the TDS protein may be expressed as an inclusion body, or targeted to a specific organelle (e.g. a plastid, vacuole, mitochondrion, nucleus, etc) to facilitate subsequent isolation. Procedures for recombinantly-expressing proteins, and for sequestering and/or purifying recombinantly-expressed proteins, are well-known to those skilled in the art. Accordingly, the present invention is not to be limited by the mode of purification of exemplified herein.

A further aspect of the present invention provides an antibody molecule prepared by a process comprising immunizing an animal with an immunologically-effective amount of an isolated TDS protein or a fragment comprising at least about 10 contiguous amino acids in length of said TDS protein, and isolating a monoclonal or polyclonal antibody from said animal.

This aspect of the invention clearly extends to any monoclonal or polyclonal antibody that binds to a TDS protein or to a fragment comprising at least about 10 contiguous amino acids in length of said TDS protein.

The term “antibody” as used herein, is intended to include fragments thereof which are also specifically reactive with a TDS protein of the present invention, or with a fragment thereof as described herein. Antibodies can be fragmented using conventional techniques and the fragments screened for utility in the same manner as for whole antibodies. For example, F(ab′)2 fragments can be generated by treating antibody with pepsin. The resulting F(ab′)2 fragment can be treated to reduce disulfide bridges to produce Fab′ fragments.

Those skilled in the art will be aware of how to produce antibody molecules when provided with the TDS protein or a fragment thereof, according to the embodiments described herein. For example, polyclonal antisera or monoclonal antibodies can be made using standard methods. A mammal, (e.g., a mouse, hamster, or rabbit) can be immunized with an immunogenic form of the polypeptide which elicits an antibody response in the mammal. Techniques for conferring immunogenicity on a polypeptide include conjugation to carriers or other techniques well known in the art. For example, the polypeptide can be administered in the presence of adjuvant. The progress of immunization can be monitored by detection of antibody titers in plasma or serum. Standard ELISA or other immunoassay can be used with the immunogen as antigen to assess the levels of antibodies. Following immunization, antisera can be obtained and, if desired IgG molecules corresponding to the polyclonal antibodies may be isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art. For example, the hybridoma technique originally developed by Kohler and Milstein (1975) as well as other techniques such as the human B-cell hybridoma technique (Kozbor et al., 1983), the EBV-hybridoma technique to produce human monoclonal antibodies (Cole et al., 1985), and screening of combinatorial antibody libraries (Huse et al., 1989). Hybridoma cells can be screened immunochemically for production of antibodies which are specifically reactive with the polypeptide and monoclonal antibodies isolated.

As with all immunogenic compositions for eliciting antibodies, the immunogenically effective amounts of the protein of the invention must be determined empirically. Factors to be considered include the immunogenicity of the native protein, whether or not the protein will be complexed with or covalently attached to a hapten, or carrier protein, or other carrier, and route of administration for the composition, i.e. intravenous, intramuscular, subcutaneous, etc., and the number of immunizing doses to be administered. Such factors are known in the vaccine art and it is well within the skill of immunologists to make such determinations without undue experimentation.

Preferably, the immunogen comprises the full-length TDS protein, or alternatively, a peptide comprising at least about 10 contiguous amino acids of the full-length polypeptide, such as, for example, an internal or N-terminal peptide fragment.

To enhance their immunogenicity, it is well-known to conjugate small peptide fragments to a hapten, such as, for example, dinitrophenyl (DNP), m-maleimidobenzoyl-N-hydroxyl-N-hybroxysuccinimide ester (MBS), or m-amino benzene sulphonate. A “hapten” is a non-immunogenic molecule that will react with a preformed antibody induced by an antigen or carrier molecule. Alternatively, the immunogenicity of small peptide fragments may be enhanced by conjugating the peptide to a carrier molecule, such as, for example, an antigenic peptide or protein, that may be conjugated to a hapten. As will be known to those skilled in the art, a “carrier” is generally an antigenic molecule. Preferred carrier molecules for this purpose include ovalbumin, KLH, and PHA.

In a particularly preferred embodiment, the immunogenic TDS protein consists of the full-length polypeptide, or a fragment thereof comprising at least 12 or at least about 30 contiguous amino acid sequences thereof.

It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody.

Immunoassays are useful in detecting the presence of a TDS protein, or synthetic peptide derivative thereof, in a cell, particularly a plant cell. Such an immunoassay is of particular use in determining whether a plant has the capability to produce condensed tannins. Immunoassays are also useful for the quantitation of said TDS protein in a cell, in particular for screening genetic stocks for breeding programmes. The invention described herein extends to all such uses of immunointeractive molecules and diagnostic assays requiring said immunoassays for their performance.

A wide range of immunoassay techniques may be such as those described in U.S. Pat. Nos. 4,016,043, 4,424,279 and 4,018,653. These methods may be employed for detecting a proanthocyanidin biosynthetic enzyme or synthetic peptide derivative thereof. For example, an antibody against the TDS protein or a synthetic peptide derivative thereof (hereinafter referred to as “the antigen”), can be immobilized onto a solid substrate to form a first complex and a biological sample derived from a test sample brought into contact with the bound antigen. After a suitable incubation, sufficient to allow formation of an antibody-antigen secondary complex, a second antibody capable of binding to the antigen and labeled with a reporter molecule is added and incubated, allowing sufficient time for the formation of a tertiary complex of antibody the antigen-labeled antibody. Any unreacted material is washed away, and the presence of the tertiary complex is determined by observation of a signal produced by the reporter molecule.

The results may either be qualitative, by simple observation of the visible signal, or they may be quantitated by comparison with a control sample containing known amounts of immunogen.

Variations of this assay include a simultaneous assay, in which both sample and labeled antibody are added simultaneously to the bound antibody, or a reverse assay in which the labeled antibody and sample to be tested are first combined, incubated and then added simultaneously to the bound antibody. These techniques are well known to those skilled in the art, and the possibility of minor variations will be readily apparent. The antibodies may be monoclonal or polyclonal.

The solid substrate is typically glass or a polymer, the most commonly used polymers being cellulose, polyacrytamide, nylon, polystyrene, polyvinyl chloride or polypropylene. The solid supports may be in the form of tubes, beads, discs or microplates, or any other surface suitable for conducting an immunoassay. The binding processes are well known in the art and generally consist of cross-linking covalently binding or physically adsorbing the molecule to the insoluble carrier.

As used herein, the term “reporter molecule” shall be taken to mean a molecule which, by its chemical nature, produces an analytically identifiable signal which allows the detection of antigen-bound antibody. Detection may be either qualitative or quantitative. The most commonly used reporter molecule in this type of assay is an enzyme, fluorophore, or radionuclide. In the case of an enzyme immunoassay, the report molecule is an enzyme, preferably conjugated to the second antibody. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable color change. It is also possible to employ fluorogenic substrates, which yield a fluorescent product.

Conjugation of a hapten, carrier, or reporter molecule, can be achieved using glutaraldehyde, or periodate. As will be readily recognized, however, a wide variety of different conjugation techniques exist which are readily available to the skilled artisan.

Alternatively, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state of excitability in the molecule, followed by emission of the light at a characteristic color visually detectable with a light microscope. As in the EIA, the fluorescent labeled antibody is allowed to bind to the first antibody-hapten complex. After washing off the unbound reagent, the remaining complex is then exposed to the light of the appropriate wavelength, the fluorescence observed indicates the presence of the hapten of interest. Immunofluorescence and EIA techniques are both very well established in the art and are particularly preferred for the present method. However, other reporter molecules, such as radioisotope, chemiluminescent or bioluminescent molecules, may also be employed. It will be readily apparent to the skilled technician how to vary the procedure to suit the required purpose.

Those skilled in the art will recognize that cross-reactive proteins (i.e. proteins that bind to anti-TDS protein antibodies) are most likely to be TDS proteins. Accordingly, the antibodies described herein are useful for isolating or purifying TDS proteins from any plant, by standard procedures of affinity purification using antibodies. Alternatively, they are used for isolating nucleic acid expressing said TDS proteins, from any source, using any art-recognized procedure. Alternatively, the antibodies can be used to immunoprecitiate or inhibit TDS protein activity present in cell extracts in vitro. Alternatively, they can be used to localize TDS protein activity in cells, such as, for example, by immunohistochemical staining of plant tissue sections.

A further aspect of the present invention provides an isolated nucleic acid molecule comprising a nucleotide sequence selected from the group consisting of:
(i) a nucleotide sequence that encodes a TDS protein or polypeptide having activity in the synthesis of proanthocyanidin (PA) polymer from epicatechin or catechin in plants, and which is not naturally regulated by the TT2 or TT8 regulators;
(ii) a nucleotide sequence that encodes a fragment comprising at least about 10 contiguous amino acids derived from said protein or polypeptide; and
(iii) a nucleotide sequence that is complementary to (i) or (ii).

The isolated nucleic acid molecule of the invention can be derived from any plant species. The present invention is not to be limited by the species origin of nucleic acid encoding the TDS protein. Without limiting the scope of the invention, preferred plant sources include those plants referred to in the index to the International Code of Botanical Nomenclature (Tokyo Code) as adopted by the Fifteenth International Botanical Congress, Yokohama, August-September 1993 (published as International Code of Botanical Nomenclature (Tokyo Code) Regnum Vegetabile 131, Koeltz Scientific Books, Königstein, ISBN 3-87429-367-X or 1-878762-66-4 or 80-901699-1-0). More preferably, the isolated nucleic acid of the invention is derived from a plant listed supra.

Even more preferably, the nucleic acid of the invention is derived from a plant selected from the group consisting of: D. uncinatum, Medicago sativa, Medicago truncatuta, Trifolium repens, Lotus corniculatus, Lotus japonicus, Nicotiana tabacum, Vitis vinifera, Camellia sinensis, Hordeum vulgare, Sorghum bicolor, Populus trichocarpa, Forsythia×intermedia, Thuja piicata, Pinus radiate, Pseudotsuga menziesii, and A. thaliana.

The nucleic acid of the invention may be in the form of RNA or DNA, such as, for example, single-stranded, double-stranded or partially double-stranded cDNA, genomic DNA, oligonucleotides, or DNA amplified by polymerase chain reaction (PCR); or a mixed polymer comprising RNA and DNA.

Nucleic acid of the present invention may be derived by organic synthesis based upon the nucleotide sequence of a naturally-occurring tds gene, or from a tds gene per se. Reference herein to a “tds gene” is to be taken in its broadest context and includes a member selected from the group consisting of:
(i) a classical genomic gene encoding all or part of a TDS protein, and consisting of transcriptional and/or translational regulatory sequences and/or a coding region and/or untranslated sequences (i.e. introns, 5′- and 3′-untranslated sequences);
(ii) mRNA or cDNA encoding all or part of a TDS protein, said mRNA or cDNA corresponding to the coding regions (i.e. exons) and 5′- and 3′-untranslated sequences of the genomic gene;
(iii) a synthetic or fusion molecule encoding all or part of a TDS protein; and
(iv) a complementary nucleotide sequence to any one of (i) to (iii).

Preferred tds genes of the present invention are derived from naturally-occurring sources using standard recombinant techniques, such as, for example, mutagenesis, to introduce single or multiple nucleotide substitutions, deletions and/or additions relative to the wild-type sequence.

It is clearly within the scope of the present invention to include any nucleic acid comprising a nucleotide sequence complementary to a tds gene as defined herein, in particular complementary nucleotide sequences that are useful as hybridization probes, or amplification primers, for isolating or identifying a tds gene, or for reducing the level of expression of an endogenous tds gene in a cell, tissue, organ, or whole plant. Such complementary nucleotide sequences may be in the form of RNA, such as, for example, antisense mRNA, or a ribozyme; DNA, such as, for example, single-stranded or double-stranded cDNA, genomic DNA, single-stranded or double-stranded synthetic oligonucleotides, or DNA amplified by polymerase chain reaction (PCR); or a mixed polymer comprising RNA and DNA. As will be known to those skilled in the art, sequences complementary to the coding region and/or non-coding region of a gene may be useful for such applications.

An antisense molecule is nucleic acid comprising a nucleotide sequence that is complementary to mRNA, or a DNA strand, that encodes protein, albeit not restricted to sequence having complementarity to the protein-encoding region. Preferred antisense molecules comprise RNA capable of hybridizing to mRNA encoding all or part of a TDS protein. Antisense molecules are thought to interfere with the translation or processing or stability of the mRNA of the target gene, thereby inactivating its expression. Methods of devising antisense sequences are well known in the art and examples of these are can be found in U.S. Pat. No. 5,190,131, European patent specification 0467349-A1, European patent specification 0223399-A1 and European patent specification 0240208, which are incorporated herein by reference. The use of antisense techniques in plants has been reviewed by Bourque (1995) and Senior (1998). Bourque lists a large number of examples of how antisense sequences have been utilized in plant systems as a method of gene inactivation. She also states that attaining 100% inhibition of any enzyme activity may not be necessary as partial inhibition will more than likely result in measurable change in the system. Senior (1998) states that antisense methods are now a very well established technique for manipulating gene expression.

Antisense molecules for TDS genes can be based on the Arabidopsis mRNA sequences or based on homologies with DNA or mRNA sequences derived from other species, for example white clover. These antisense sequences may correspond to the structural genes or for sequences that effect control over the gene expression or splicing event. For example, the antisense sequence may correspond to the targeted coding region of the gene or to the 5′-untranslated region (UTR) or the 3′-UTR or combination of these. It may be complementary in part to Intron sequences, which may be spliced out during or after transcription, preferably only to exon sequences of the target gene. In view of the generally greater divergence of the UTRs, targeting these regions provides greater specificity of gene inhibition. The length of the antisense sequence should be at least 19 contiguous nucleotides, preferably at least 30 or 50 nucleotides, and more preferably at least 100, 200, 500 or 1000 nucleotides. The full-length sequence complementary to the entire gene transcript may be used. The length is most preferably 100-2000 nucleotides. The degree of homology of the antisense sequence to the targeted transcript should be at least 85%, preferably at least 90% and more preferably 95-100%. The antisense RNA molecule may of course comprise unrelated sequences which may function to stabilize the molecule.

In the present context, a “ribozyme” is a synthetic RNA molecule which comprises one or preferably two hybridizing sequences, each of at least about 5-20 contiguous nucleotides in length, capable of hybridizing to mRNA encoding a TDS protein, and possessing an endoribonuclease activity that is capable of catalytically cleaving said mRNA. Ribozymes can cleave the mRNA molecules at specific sites defined by the hybridizing sequences. The cleavage of the RNA inactivates the expression of the target gene. The ribozymes may also act as an antisense molecule, which may contribute to the gene inactivation. The ribozymes contain one or more catalytic domains, preferably of the hammerhead or hairpin type, between the hybridizing sequences. Other ribozyme motifs may be used including RNAseP, Group I or II introns, and hepatitis delta virus types. Reference is made to European patent specification 0321201 and U.S. Pat. No. 6,221,661. The use of ribozymes to inactivate genes in transgenic plants has been demonstrated. As with antisense molecules, ribozymes may target regions in the mRNA other than those of the protein-encoding region, such as, for example, in the untranslated region of a tds gene.

The term “untranslated region” in this context means a region of a genomic gene or cDNA that is normally transcribed in a cell but not translated into an amino acid sequence of a TDS protein. Accordingly, the term “untranslated region” includes nucleic acid comprising a nucleotide sequence derived from the 5′-end of mRNA to immediately preceding the ATG translation start codon; nucleic acid comprising a nucleotide sequence from the translation stop codon to the 3′-end of mRNA; and any intron sequence that is cleaved from a primary mRNA transcript during mRNA processing.

The present invention further encompasses within its scope nucleic acid molecules comprising a first sense nucleotide sequence derived from mRNA, or a DNA strand, encoding a TDS protein, and a second antisense nucleotide sequence complementary to mRNA encoding a TDS protein, such as for example, in the form of a post-transcription gene silencing (PTGS) molecule. The first and second sequences may be linked in head-to-head or tail-to-tail (inverted) configuration. As with antisense molecules or ribozymes, such molecules need not be derived exclusively from the open reading frame of a tds gene. Sequences derived from untranslated regions, in particular the 5′ or 3′ untranslated regions, may be preferred for the sense nucleotide sequence. Preferred PTGS molecules will have a region of self-complementarity and be capable of forming a hairpin loop structure, such as those described in International Patent Application No. PCT/IB99/00606. Whilst not being bound by any theory or mode of action, a PTGS molecule has the potential to sequester sense tds-encoding mRNA in a cell, such that the sequestered mRNA is degraded. In a preferred embodiment, the sense and antisense sequences are separated by a spacer region that comprises an intron which, when transcribed into RNA, is spliced out. This arrangement has been shown to result in a higher efficiency of gene silencing (Smith et al., 2000). The double-stranded RNA region may comprise one or two or more RNA molecules, transcribed from either one DNA region or two or more. The presence of the double stranded molecule is thought to trigger a response from an endogenous plant system that destroys both the double stranded RNA and also the homologous RNA transcript from the target plant gene, efficiently reducing or eliminating the activity of the target gene. The length of the sense and antisense sequences that hybridise should each be at least 19 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of homology of the sense and antisense sequences to the targeted transcript should be at least 85%, preferably at least 90% and more preferably 95-100%. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters such as a U6 promoter.

The antisense, cosuppression or double stranded RNA molecules may also comprise a largely double-stranded RNA region, preferably comprising a nuclear localization signal, as described in PCT/AU03/00292. In a preferred embodiment, the largely double-stranded region is derived from a PSTVd type viroid or comprises at least 35 CUG trinucleotide repeats.

Preferred nucleic acid encoding a TDS protein will be in the form of sense nucleic acid. In the present context, the term “sense nucleic acid” shall be taken to mean RNA or DNA comprising a nucleotide sequence derived from the strand of DNA or RNA that encodes a full-length TDS protein, or a part thereof, including both coding and non-coding sequences. As will be known to those skilled in the art, sense nucleic acid may be used to for the purposes of ectopically expressing mRNA, or protein, in a cell, or alternatively, to down-regulate expression (e.g. co-suppression), or to Identify or isolate a tds gene, or to identify or isolate complementary sequences, such as, for example, antisense mRNA. As will be known to those skilled in the art, “co-suppression” is the reduction in expression of an endogenous gene that occurs when one or more copies of said gene, or one or more copies of a substantially similar gene, or fragments thereof, are introduced into the cell. The mechanism of co-suppression is not well understood but is thought to involve post-transcriptional gene silencing (PTGS) and in that regard may be very similar to many examples of antisense suppression or duplex RNA suppression. It involves P introducing an extra copy of a gene or a fragment thereof into a plant in the sense orientation with respect to a promoter for its expression. The size of the sense fragment, its correspondence to target gene regions, and its degree of homology to the target gene are as for the antisense sequences described above. In some instances the additional copy of the gene sequence interferes with the expression of the target plant gene. Reference is made to Patent specification WO 97/20936 and European patent specification 0465572 for methods of implementing co-suppression approaches. As will be known to those skilled in the art, whilst the coding region of a gene is required to ectopically-express protein in a cell, the coding region and/or non-coding region of a gene may be useful for other applications referred to herein.

Sense nucleic acid molecules will preferably comprise the full-length open reading frame of an endogenous tds gene, however may be less than full-length. It will be apparent from the definition of the term “tds gene” provided herein above, that the present invention encompasses within its scope any nucleic acid fragment of the full-length open reading frame of a tds gene, that is at least useful as a hybridization probe or amplification primer for isolating a tds gene, or for modifying the level of expression of an endogenous tds gene.

Preferred fragments of a tds gene of the invention, for isolating or identifying homologous genes in the same or another species, are derived from the open reading frame. In the present context, an “open reading frame” is any nucleotide sequence encoding an amino acid sequence of a TDS protein, and preferably, at least about 10 contiguous amino acids of a TDS protein.

As will be known to those skilled in the art, where homologous tds gene sequences are from divergent species to the species from which the fragment is derived, fragments of at least about 20 nucleotides in length from within the open reading frame of the tds gene, more preferably at least about 30-50 nucleotides in length, and more preferably at least about 100 nucleotides in length, or 500 nucleotides in length, are preferred.

In the case of fragments for isolating or identifying an identical target tds gene, or a tds gene from a closely-related species, the fragment may be derived from any part of a known tds gene, such as, for example, from the open reading frame, an untranslated region, or an intron, or promoter sequence.

In the present context, the term “promoter” means a nucleotide sequence comprising a transcriptional regulatory sequence for initiation of transcription, such as, for example, the TATA box which is required for accurate transcription initiation, with or without a CCAAT box sequence and additional cis-acting regulatory elements (i.e. upstream activating sequences, enhancers and silencers). Preferred promoters are those derived from a tds gene, or those that may alter tds gene expression in response to developmental and/or external stimuli, or in a tissue-specific manner.

Preferably, a nucleotide sequence that encodes a TDS protein or a complementary nucleotide sequence thereto is selected from the group consisting of:
(I) a nucleotide sequence having at least about 40% identity overall to a SEQ ID NO: 1 or SEQ ID NO: 3, or to a coding region thereof;
(ii) a nucleotide sequence that encodes a protein or polypeptide having at least about 40% Identity overall to SEQ ID NO: 2 or SEQ ID NO: 4;
(iii) a nucleotide sequence that encodes a fragment comprising at least about 10 contiguous amino acids derived from the protein or polypeptide of (II);
(iv) a nucleotide sequence that hybridizes under at least low stringency conditions to at least about 20 contiguous nucleotides of anyone of (i) to (III); and
(v) a nucleotide sequence that is complementary to any one of (i) to (iv).

Preferably, the percentage identity overall to a nucleotide sequence presented herein is at least about 50%, more preferably at least about 60%, even more preferably at least about 70%, and even more preferably, at least about 80%, and still even more preferably at least about 90%. In preferred embodiments, the invention provides nucleotide sequences which have at least 40%, 50%, 60%, 70%, 80% or even 90% nucleotide sequence identity to the coding region of SEQ ID NO: 1 or SEQ ID NO: 3.

Similarly, it is preferred for the percentage identity overall to an amino acid sequence presented herein, is at least about 40%, more preferably about 50%, even more preferably at least about 60%, and even more preferably at least about 70%, and still even more preferably at least about 80%, and even more preferably at least about 90%.

For the purposes of defining the level of stringency in a hybridization to any one of the nucleotide sequences disclosed herein, a low stringency hybridization may comprise a hybridization and/or a wash carried out using a salt concentration equivalent to SSC buffer in the range of 2×SSC to 6×SSC buffer; a detergent concentration in the range of 0.1% (w/v) SDS to 1% (w/v) SDS; and a temperature in the range of between ambient temperature to about 42° C. Those skilled in the art will be aware that several different hybridization conditions may be employed. For example, Church buffer may be used at a temperature in the range of between ambient temperature to about 45° C.

Preferably, the stringency of hybridization is at least moderate stringency, even more preferably at high stringency. Generally, the stringency is increased by reducing the concentration of SSC buffer, and/or increasing the concentration of SDS in the hybridization buffer or wash buffer and/or increasing the temperature at which the hybridization and/or wash are performed. Conditions for hybridizations and washes are well understood by one normally skilled in the art. For example, a moderate stringency hybridisation may comprise a hybridization and/or wash carried out using a salt concentration in the range of between about 1×SSC buffer and 2×SSC buffer; a detergent concentration of up to about 0.1% (w/v) SDS; and a temperature in the range of about 45° C. to 55° C. Alternatively, Church buffer may be used at a temperature of about 55° C., to achieve a moderate stringency hybridization. A high stringency hybridisation may comprise a hybridization and/or wash using a salt concentration in the range of between about 0.1×SSC buffer and about 1×SSC buffer, a detergent concentration of about 0.1% (w/v) SDS; and a temperature of about 55° C. to about 65° C., or alternatively, a Church Buffer at a temperature of at least 65° C. Variations of these conditions will be known to those skilled in the art.

Clarification of the parameters affecting hybridization between nucleic acid molecules, is provided by Ausubel et al. (1987).

Although the present inventors have successfully isolated the tds gene using oligonucleotide primers of only about 20 nucleotides in length, those skilled in the art will recognize that the specificity of hybridization increases using longer probes, or primers, to detect genes in standard hybridization and PCR protocols. Such approaches are facilitated by the provision herein of full-length cDNAs from a number of diverse species. For example, persons skilled in the art are readily capable of aligning the nucleotide sequences or amino acid sequences provided herein to identify conserved regions thereof, to facilitate the identification of sequences from other species or organisms. For example, conserved regions of the TDS protein may facilitate the preparation of a hybridization probe, or primer, comprising at least about 30 nucleotides in length. Accordingly, preferred nucleotide sequences according to this embodiment of the invention will hybridize to at least about 30 contiguous nucleotides, more preferably at least about 50 contiguous nucleotides, even more preferably at least about 100 contiguous nucleotides, and still even more preferably at least about 500 contiguous nucleotides.

In a particularly preferred embodiment, the nucleic acid of the invention comprises the sequence set forth in SEQ ID NO: 1 or SEQ ID NO: 3, a coding region thereof, or a sequence complementary thereto.

The present invention clearly encompasses within its scope those nucleic acid molecules from organisms other than those plants specifically described herein that encode TDS proteins, and have sequence homology to the exemplified sequences of the invention. Accordingly, in a further embodiment, the present invention provides an isolated nucleic acid molecule comprising a nucleotide sequence that encodes a TDS protein or a fragment thereof, wherein said nucleic acid molecule is isolated by a process comprising:
(1) hybridizing a probe or primer comprising at least about 20 contiguous nucleotides of SEQ ID NO: 1 or SEQ ID NO: 3 or a degenerate or complementary nucleotide sequence thereto, to nucleic acid of plants;
(ii) detecting said hybridization;
(iii) isolating the hybridized nucleic acid; and
(iv) determining the amino acid sequence encoded by the hybridized nucleic acid or the function of said amino acid sequence so as to determine that the hybridized nucleic acid encodes said TDS protein.

The use of probes or primers encoding fragments of the amino acid sequence set forth in SEQ ID NO: 2 or SEQ ID NO: 4 are also contemplated herein, the only requirement being that such probes or primers are capable of hybridizing to a tds gene.

The related sequence being identified may be present in a gene library, such as, for example, a cDNA or genomic gene library.

The library may be any library capable of maintaining nucleic acid of eukaryotes, such as, for example, a BAG library, YAC library, cosmid library, bacteriophage library, genomic gene library, or a cDNA library. Methods for the production, maintenance, and screening of such libraries with nucleic acid probes or primers, or alternatively, with antibodies, are well known to those skilled in the art. The sequences of the library are usually in a recombinant form, such as, for example, a cDNA contained in a virus vector, bacteriophage vector, yeast vector, baculovirus vector, or bacterial vector. Furthermore, such vectors are generally maintained in appropriate cellular contents of virus hosts.

In particular, cDNA may be contacted, under at least low stringency hybridization conditions or equivalent, with a hybridization-effective amount of a probe or primer.

In one embodiment, the detection means is a reporter molecule capable of giving an identifiable signal (e.g. a radioisotope such as ³²P or ³⁵S or a biotinylated molecule) covalently linked to the isolated nucleic acid molecule of the invention. Conventional nucleic acid hybridization reactions, such as, for example, those described by Ausubel et al., are encompassed by the use of such detection means.

In an alternative method, the detection means is any known format of the polymerase chain reaction (PCR). According to this method, degenerate pools of nucleic acid “primer molecules” of about 20-50 nucleotides in length are designed based upon any one or more of the nucleotide sequences disclosed herein, or a complementary sequence thereto, in one approach related sequences (i.e. the “template molecule”) are hybridized to two of said primer molecules, such that a first primer hybridizes to a region on one strand of the double-stranded template molecule and a second primer hybridizes to the other strand of said template, wherein the first and second primers are not hybridized within the same or overlapping regions of the template molecule and wherein each primer is positioned in a 5′- to 3′-orientation relative to the position at which the other primer is hybridized on the opposite strand. Specific nucleic acid molecule copies of the template molecule are amplified enzymatically, in a polymerase chain reaction (PCR), a technique that is well known to one skilled in the art. McPherson et al (1991) describes several formats of PCR.

The primer molecules may comprise any naturally occurring nucleotide residue (i.e. adenine, cytidine, guanine, and thymidine) and/or comprise inosine or functional analogues or derivatives thereof, capable of being incorporated into a polynucleotide molecule. The nucleic acid primer molecules may also be contained in an aqueous mixture of other nucleic acid primer molecules or be in a substantially pure form.

Preferably, the sequence detected according to this embodiment originates from a plant as listed supra.

The present invention clearly extends to any gene constructs that comprise the isolated nucleic acid molecule of the present invention, such as, for example, any expression gene constructs produced for expressing said nucleic acid molecule in a bacterial, insect, yeast, plant, fungal, or animal cell.

Accordingly, a further aspect of the present invention is directed to a gene construct comprising an isolated nucleic acid that encodes a TDS protein or a biologically active fragment thereof, or complementary nucleotide sequence thereto. The invention also provides a gene construct encoding an inhibitory molecule such as, for example, an antisense, ribozyme, PTGS or co-suppression molecule that is capable of inhibiting tds gene activity in a cell. In a preferred embodiment, the invention provides a chimeric gene construct in which the coding region encoding a TDS protein or a biologically active fragment thereof is capable of being expressed from a promoter that does not naturally control expression of the TDS protein (heterologous promoter).

Those skilled in the art will also be aware that expression of a tds gene, or a complementary sequence thereto, in a cell, requires said gene to be placed in operable connection with a promoter sequence. The choice of promoter for the present purpose may vary depending upon the level of expression required and/or the tissue, organ and species in which expression is to occur.

References herein to placing a nucleic acid molecule under the regulatory control of a promoter sequence mean positioning said molecule such that expression is controlled by the promoter sequence. A promoter is usually, but not necessarily, positioned upstream, or at the 5′-end, of the nucleic acid molecule it regulates. Furthermore, the regulatory elements comprising a promoter are usually positioned within 2 kb of the start site of transcription of the gene. In the construction of heterologous promoter/structural gene combinations, it is generally preferred to position the promoter at a distance from the gene transcription start site that is approximately the same as the distance between that promoter and the gene it controls in its natural setting (i.e., the gene from which the promoter is derived). As is known in the art, some variation in this distance can be accommodated without loss of promoter function. Similarly, the preferred positioning of a regulatory sequence element with respect to a heterologous gene to be placed under its control is defined by the positioning of the element in its natural setting (i.e., the gene from which it is derived). Again, as is known in the art, some variation in this distance can also occur.

Examples of promoters suitable for use in gene constructs of the present invention include promoters derived from the genes of viruses, yeast, moulds, bacteria, insects, birds, mammals and plants, preferably those capable of functioning in isolated yeast or plant cells. The promoter may regulate expression constitutively, or differentially, with respect to the tissue in which expression occurs. Alternatively, expression may be differential with respect to the developmental stage at which expression occurs, or in response to external stimuli such as physiological stresses, or temperature.

Examples of promoters useful for expression in plants include the CaMV 35S promoter, NOS promoter, octopine synthase (OCS) promoter, Arabidopsis thaliana SSU gene promoter, the meristem-specific promoter (meri1), napin seed-specific promoter, actin promoter sequence, sub-clover stunt virus promoters (International Patent Application No. PCT/AU95/00552), and the like. In addition to the specific promoters identified herein, cellular promoters for so-called housekeeping genes are useful. Promoters derived from genomic gene equivalents of the cDNAs described herein are particularly contemplated for regulating expression of tds genes, or complementary sequences thereto, in plants. Inducible promoters, such as, for example, a heat shock-inducible promoter, heavy metal-inducible promoter (e.g. metallotheinin gene promoter), ethanol-inducible promoter, or stress-inducible promoter, may also be used to regulate expression of the introduced nucleic acid of the invention under specific environmental conditions.

For certain applications, it is preferable to express the tds gene of the invention specifically in particular tissues of a plant, such as, for example, to avoid any pleiotropic effects that may be associated with expressing sak) gene throughout the plant in particular, the tds gene may be expressed in a tissue-specific manner in parts or tissues of the plant in which the gene is not expressed in wild type plants, for example in the leaves or stems or seeds or storage organs of the plant. As will be known to the skilled artisan, tissue-specific or cell-specific promoter sequences may be required for such applications. For expression in particular plant tissues, reference is made to the publicly available or readily available sources of promoter sequences known to those skilled in the art.

For expression in yeast or bacterial cells, it is preferred that the promoter is selected from the group consisting of: GAL1, GAL10, CYC1, CUP1, PGK1, ADH2, PHOS, PRB1, GUT1, SP013, ADH1, CMV, SV40, LACZ, T3, SP6, T5, and 77 promoter sequences.

The gene construct may further comprise a terminator sequence and be introduced into a suitable host cell where it is capable of being expressed to produce a recombinant dominant-negative polypeptide gene product or alternatively, a co-suppression molecule, a ribozyme, gene silencing or antisense molecule.

The term “terminator” refers to a DNA sequence at the end of a transcriptional unit which signals termination of transcription. Terminators are 3′-non-translated DNA sequences containing a polyadenylation signal, which facilitates the addition of poly(A) sequences to the 3″-end of a primary transcript.

Terminators active in cells derived from viruses, yeast, moulds, bacteria, insects, birds, mammals and plants are known and described in the literature. They may be isolated from bacteria, fungi, viruses, animals and/or plants.

Examples of terminators particularly suitable for use in the gene constructs of the present invention include the nopaline synthase (NOS) gene terminator of Agrobacterium tumefaciens, the terminator of the Cauliflower mosaic virus (CaMV) 35S gene, the zein gene terminator from Zea mays, the Rubisco small subunit (SSU) gene terminator sequences, subclover stunt virus (SCSV) gene sequence terminators (International Patent Application No. PCT/AU95/00552), and the terminator of the Flavaria bidentis malic enzyme gene meA3 (International Patent Application No. PCT/AU95/00552).

Those skilled in the art will be aware of additional promoter sequences and terminator sequences suitable for use in performing the invention. Such sequences may readily be used without any undue experimentation.

The gene constructs of the invention may further include an origin of replication sequence which is required for replication in a specific cell type, for example a bacterial cell, when said gene construct is required to be maintained as an episomal genetic element (e.g. plasmid or cosmid molecule) in said cell.

Preferred origins of replication for use in bacterial cells include, but are not limited to, the f1-ori and colE1 origins of replication. The 2-micron origin of replication may be used in gene constructs for use in yeast cells.

The gene construct may further comprise a selectable marker gene or genes that are functional in a cell into which said gene construct is introduced. As used herein, the term “selectable marker gene” includes any gene which confers a phenotype on a cell in which it is expressed to facilitate the identification and/or selection of cells which are transfected or transformed with a gene construct of the invention or a derivative thereof.

Suitable selectable marker genes contemplated herein include the ampicillin resistance (Amp^{r}), tetracycline resistance gene (Tc^{r}), bacterial kanamycin resistance gene (Kan^{r}), phosphinothricin resistance gene, neomycin phosphotransferase gene (nptll), hygromycin resistance gene, β-glucuronidase (GUS) gene, chloramphenicol acetyltransferase (CAT) gene and luciferase gene, amongst others.

In a preferred embodiment of the invention, the gene construct is a binary gene construct, more preferably a binary gene construct comprising a selectable marker gene selected from the group consisting of: bar, nptll and spectinomycin resistance genes. Those skilled in the art will be aware of the chemical compounds to which such selectable marker genes confer resistance.

In an even more preferred embodiment, the binary construct comprises the Streptomyces hygroscopicus bar gene, placed operably in connection with the CaMV 35S promoter sequence. Still more preferably, the binary construct comprises the Streptomyces hygroscopicus bar gene, placed operably in connection with the CaMV 35S promoter sequence and upstream of the terminator sequence of the octopine synthase (ocs) gene.

A further aspect of the invention contemplates an isolated cell comprising a heterologous tds gene, preferably wherein said tds gene is present in said cell in an expressible format.

As used herein, the word “cell” shall be taken to include an isolated cell, or a cell contained within organized tissue, a plant organ, or whole plant.

Preferably the cell is a bacterial cell, such as, for example, E. coli or A. tumefaciens, or a plant cell, such as a legume, more particularly a fodder or forage legume such as Medicago spp. and Trifolium spp. Even more preferably, the cell is an Agrobacterium tumefaciens strain carrying a disarmed Ti plasmid, such as, for example, the Agrobacterium tumefaciens strain is designated AGL1 (Lazo et al., 1991). However, as will be understood by those skilled in the art, the isolated nucleic add of the present invention may be introduced to any cell and maintained or replicated therein, for the purposes of generating probes or primers, or to produce recombinant TDS protein, or a fragment thereof. Accordingly, the present invention is not limited by the nature of the cell.

Those skilled in the art will be aware that whole plants may be regenerated from individual transformed cells. Accordingly, the present invention also extends to any plant material which comprises a gene construct according to any of the foregoing embodiments or expresses a sense, antisense, ribozyme, PTGS or co-suppression molecule, and to any cell, tissue, organ, plantlet or whole plant derived from said material.

A further aspect of the invention contemplates a transformed plant comprising a non-endogenous tds gene or fragment thereof introduced into its genome, or a nucleotide sequence that is complementary to said tds gene or said fragment, in an expressible format. The non-endogenous tds gene includes genes in which a TDS coding region that is endogenous to the plant is operably under the control of a non-endogenous promoter.

The term “endogenous” as used herein refers to the normal complement of a stated integer which occurs in an organism in its natural setting or native context (i.e. in the absence of any human intervention, in particular any genetic manipulation).

The term “non-endogenous” as used herein shall be taken to indicate that the stated integer is derived from a source which is different to the plant material, plant cell, tissue, organ, plantlet or whole plant into which it has been Introduced. The term “non-endogenous” shall also be taken to include a situation where genetic material from a particular species is introduced, in any form, into an organism belonging to the same species as an addition to the normal complement of genetic material of that organism.

Preferably, the transformed plant of the invention further expresses a non-endogenous TDS protein. This aspect of the invention clearly extends to any plant parts, or progeny plants, that are derived from the primary transformed plant.

Preferably, the plant, material, plant cell, tissue, organ, plantlet or whole plant comprises or is derived from a fodder crop, companion plant, food crop, tree, shrub or ornamental plant as described herein, or a tissue, cell or organ culture of any of said plants or the seeds of any of said plants, in particular a legume, more particularly a fodder and forage legume such as Medicago spp. and Trifolium spp.

The present invention extends to the progeny and clonal derivatives of a plant according to any one of the embodiments described herein.

As will be known those skilled in the art, transformed plants are generally produced by introducing a gene construct, or vector, into a plant cell, by transformation or transfection means. The isolated nucleic acid molecule of the invention, especially the tds gene of the invention, or a gene construct comprising same, is introduced into a cell using any known method for the transfection or transformation of a plant cell. Wherein a cell is transformed by the gene construct of the invention, a whole plant may be regenerated from a single transformed cell, using methods known to those skilled in the art.

By “transfect” is meant that the tds gene or a PTGS molecule, antisense molecule, co-suppression molecule, or ribozyme comprising sequences derived from the tds gene, is introduced into a cell without integration into the cell's genome. Alternatively, a gene construct comprising said gene, said molecule, or said ribozyme, placed operably under the control of a suitable promoter sequence, can be used.

By “transform” is meant the tds gene or a PTGS molecule, antisense molecule, co-suppression molecule, or ribozyme comprising sequences derived from the tds gene, is introduced into a cell and integrated into the genome of the cell. Alternatively, a gene construct comprising said gene, said molecule, or said ribozyme, placed operably under the control of a suitable promoter sequence, can be used.

Means for introducing recombinant DNA into plant cells or tissue include, but are not limited to, direct DNA uptake into protoplasts, PEG-mediated uptake to protoplasts, electroporation, microinjection of DNA, microparticle bombardment of tissue explants or cells, vacuum-infiltration of tissue with nucleic acid, and T-DNA-mediated transfer from Agrobacterium to the plant tissue. All of these techniques are well known in the art.

For example, transformed plants can be produced by the method of in plants transformation method using Agrobacterium tumefaciens, wherein A. tumefaciens is applied to the outside of the developing flower bud and the binary vector DNA is then introduced to the developing microspore and/or macrospore and/or the developing seed, so as to produce a transformed seed. Those skilled in the art will be aware that the selection of tissue for use in such a procedure may vary, however it is preferable generally to use plant material at the zygote formation stage for in planta transformation procedures.

A method for the efficient introduction of genetic material into Trifolium repens and regeneration of whole plants therefrom is also described in International Patent Application No. PCT/AU97/00529, Voisey et al (1994), or Larkin et al., (1996).

Alternatively, microparticle bombardment of cells or tissues may be used, particularly in cases where plant cells are not amenable to transformation mediated by A. tumefaciens. In such procedures, microparticle is propelled into a cell to produce a transformed cell. Any suitable biolistic cell transformation methodology and apparatus can be used in performing the present invention. Stomp et al. (U.S. Pat. No. 5,122,466) or Sanford and Wolf (U.S. Pat. No. 4,945,050) discloses exemplary apparatus and procedures. When using biolistic transformation procedures, the genetic construct may incorporate a plasmid capable of replicating in the cell to be transformed. Exemplary microparticles suitable for use in such systems include 1 to 5 micron gold spheres. The DNA construct may be deposited on the microparticle by any suitable technique, such as by precipitation.

A whole plant may be regenerated from the transformed or transfected cell, in accordance with procedures well known in the art. Plant tissue capable of subsequent clonal propagation, whether by organogenesis or embryogenesis, may be transformed with a gene construct of the present invention and a whole plant regenerated therefrom. The particular tissue chosen will vary depending on the clonal propagation systems available for, and best suited to, the particular species being transformed. Exemplary tissue targets include leaf disks, pollen, embryos, immature embryos, scutellum, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The term “organogenesis”, as used herein means a process by which shoots and roots are developed sequentially from a meristematic center.

The term “embryogenesis”, as used herein, means a process by which shoots and roots develop together in a concerted fashion (not sequentially), whether from somatic cells or gametes.

The generated transformed plants may be propagated by a variety of means, such as by clonal propagation or classical breeding techniques. For example, a first generation (or T1) transformed plant may be selfed to give homozygous second generation (or T2) transformant and the T2 plants further propagated through classical breeding techniques.

The generated transformed organisms contemplated herein may take a variety of forms. For example, they may be chimeras of transformed cells and non-transformed cells; clonal transformants (e.g., all cells transformed to contain the expression cassette), grafts of transformed and untransformed tissues (e.g., in plants, a transformed rootstock grafted to an untransformed scion).

The nucleic acid of the invention, and gene constructs comprising same, are particularly useful for modifying levels of condensed tannins in plants. In this respect, the isolated nucleic acid of the invention placed in either the sense or the antisense orientation relative to a suitable promoter sequence, wherein said orientation will depend upon the desired end-result for which the gene construct is intended. The levels of condensed tannins in the plant may be increased or decreased, in parts of the plant or throughout the plant, or increased in at least one tissue and decreased in at least one other tissue, for example increased in the aerial growing parts of a plant but decreased in seed.

Such plants may exhibit a range of desired traits including, but not limited to improved bloat-safety for animals grazing thereupon (i.e. less propensity to induce bloating when ingested), increased efficiency of protein utilization in ruminants with concomitant higher productivity, improved disease- or pest-resistance.

As used herein, “higher productivity” shall be taken to refer to increased production in any biological product or secondary metabolite of an animal species, in particular a livestock animal selected from the list comprising sheep, goats, alpaca, cattle, dairy cattle, amongst others, which is at least partly attributable to said animal being grazed upon or otherwise fed a plant comprising a gene construct of the present invention. Preferably, higher productivity includes increased milk yield, increased meat production or increased wool production.

Food plants comprising higher levels of condensed tannins, which have been produced using the gene constructs of the present invention, afford the benefit of having a longer shelf life than otherwise. Whilst not being bound by any theory or mode of action, the longer shelf life of such food plants is due to the antioxidant and antimicrobial properties of condensed tannins. These effects also provide for the development of new and improved health foods or other foodstuffs with improved anti-oxidant activities and free radical scavenging properties, which are useful in the treatment or prevention of a range of diseases including, but not limited to cancer, rheumatoid arthritis or other inflammatory diseases.

For example, the introduction of additional copies of a tds gene, in the sense orientation, and under the control of a strong promoter, is useful for the production of plants, in particular fodder and forage legumes, which exhibit increased condensed tannin content or more rapid rates of condensed tannin biosynthesis. In this regard, the present inventors have produce TDS protein sequences capable of expressing a functional TDS protein useful for such an application.

Alternatively, gene constructs comprising an tds gene in the sense orientation may be used to complement the existing range of proanthocyanidin genes present in a plant, thereby altering the composition or timing of deposition of condensed tannins. In a preferred embodiment, the proanthocyanidin gene from one plant species is used to transform a plant of a different species, thereby introducing novel proanthocyanidin biosynthetic metabolism to the second-mentioned plant species.

Furthermore, the gene constructs of the invention which express an active TDS protein may be introduced into non-legume companion species which serve as companion plants for bloat-inducing fodder and forage legumes such as lucerne (alfalfa) or white clover. In this embodiment, when the levels of condensed tannins in the companion species are sufficiently high, the bloat-safe companion species counters the action of the bloat-inducing forage-legume when both crops are ingested by a grazing animal. Preferred companion plants include, but are not limited to several species of Lolium, in particular L. perenne.

In a further embodiment, the rate of condensed tannin deposition may be reduced leading to a reduction in the total tannin content of plants by transferring one or more antisense, ribozyme, PTGS, or co-suppression molecules into a plant using a suitable gene construct as a delivery system.

The benefits to be derived from reducing tannin content in plants are especially apparent in fodder crops such as, but not limited to Onobrychis viciifolia, Onithopus pinnatus, Omithpus compressus, Coronilla varia, Lotus corniculatus, Lotus pedunculatus, Lotus purshianus, Lotus angustissimus, Lotus tenuis, Lespediza stipulacea, Desmodium intortum, Desmodium uncinatum, Leucaena leococephala, Macrotyloma axillare, Stylosanthes gracilis, Trifolium dubium, Hordeum vulgare, Vitis vinifera, Calliandra spp, Arachis spp, Brachiaria spp., Codariocalyx spp, Gliricidia spp, Erythrina spp, Flemingia spp, Phyllodium spp., Tadehegi spp. or Dioclea spp., amongst others, where improved palatability or digestibility of said crop is desired.

Benefits are also to be derived in the brewing industry, from reducing the levels of condensed tannins present in barley crops. In particular, the presence of condensed tannins is undesirable in barley seed as it produces hazes in the brewed product, which is currently removed at great cost by filtration means.

The present invention is further described in the following non-limiting Examples. The examples herein are provided for the purposes of exemplification only and should not be taken as an intention to limit the subject invention.

### EXAMPLES

### Example 1

### Materials and Methods

### Materials

Authentic standards of kaempferol, quercetin, myrcetin, naringenin, pelargonidin, cyanidin, delphinidin, catechin and epicatechin were purchased from commercial suppliers. Leucocyanidin was prepared using published methods (Tanner and Kristiansen, 1993).

### PA Mutant Screen.

Seed pools from the Feldmann (6500 T-DNA insertion lines), INRA (second and third set, 3900 lines) and Weigel (first set, 8600 lines) sets of mutants, available through the Arabidopsis Biological Resource Centre (Ohio State University, Columbus), each consisting of pooled seed from 100 T-DNA tagged lines, were screened in the first round. Seed were stained with DMACA reagent (2% dimethylaminocinnamaldehyde (DMACA), in 3M HCl/50% methanol) for one week, and then washed three times with 70% (v/v) ethanol. The stained pools were then examined for seed showing altered proanthocyanidin (PA) expression using a microscope. Wild-type seed stain dark brown under this treatment by reaction of DMACA with PA. A second round of screening consisted of staining 5 pools each of 20 lines that made up the initial pool of 100. When mutant seeds were seen in these two rounds of screening, plants were then grown from selected pools of 20 lines. Seed were sterilised using 0.1% (w/v) mercuric chloride for 15 min, washed three times with H₂O, germinated on MS medium and then transferred to soil after two weeks. Plants were grown in 16 h day at 22° C. and 8 h night at 18° C. in a growth cabinet Whole siliques were stained with DMACA reagent and the individual PA-free or PA-altered mutants from the seed pools identified. The tds6-2 and ff2 seeds corresponded to the Salk_{—}096551 and SALK_{—}005260 lines. Insertion mutant information was obtained from the SIGnAL website at http://signal.salk.edu.

### Genetic Analysis of Mutants

The mutant plants were backcrossed with wild-type plants of A. thaliana ecotypes Col-7, Ws-2 or Ws-4. After selfing the F₁ plants, F₂ seed were collected and stained with DMACA to examine the PA phenotype. After selfing the F2 plants, F3 seed were collected and stained with DMACA to determine segregation of the mutant phenotype. The same process was used for crosses between mutants to determine allelism. Samples of F2 seed were also germinated on MS containing either kanamycin (50 μg/mL) or Basta (5 μg/mL) to assess the segregation of marker genes (nptll or bar) present on the T-DNA. Samples of seed from ban (accession F36), tt1, tt2, tt3, tt7 and tt8 mutants (accessions cs82, cs83, cs84, cs88 and cs111, respectively) and other A. thaliana were obtained from Arabidopsis Biological Resource Centre.

### Anthocyanin and PA Extraction

Leaves and developing siliques collected from at least 10 plants of each type were frozen in liquid N2 and stored at −80° C. Samples were ground in liquid N2 and anthocyanin and PA extracted using 1% HCl in methanol or 70% acetone containing 0.1% ascorbate, respectively, for 16-18 hours at 4° C. This was repeated 2 times, for 2 hours each extraction. The crude anthocyanin preparations were extracted further using Folch partitioning (Folch et al., 1951) with chloroform/H₂O to remove chlorophyll (×2), and then extracted with hexane (×2). To simplify interpretation of chromatograms, glycosides were removed by acid hydrolysis and the free aglycones examined. Samples were hydrolysed by adding an equal volume of 37% HCl and boiling for 15 min. Boiled samples were then extracted into pentan-2-ol, which was evaporated under vacuum centrifugation. Samples were dissolved in 1% HCl in methanol, spotted onto 0.1 mm cellulose TLC plates (Merck), and developed using A & F #9 (HCl: formic acid: H₂O 19:40:41 v/v/v) (Andersen and Francis, 1985). Dried plates were sprayed with 1% methanolic diphenylboryloxyethylamine (NP stain), followed by 5% ethanolic polyethylene glycol 4000 and then analyzed for anthocyanins and flavonols. Images of the plates were recorded in visible light with an HPScanJet 4C/T scanner or photographed under UV illumination at 310 and 365 nm.

The acetone fraction of PA extracts was treated with ethyl acetate to partition the monomers and small oligomers into the ethyl acetate phase from PA polymers which remain in the aqueous phase (Nonaka et al., 1983, 1985). Both fractions were then extracted with hexane (×3) and then chloroform. The ethyl acetate fractions were spotted directly onto cellulose TLC plates, and developed using sBAWC (s-butanol: H₂O: acetic acid: chloroform 70:20:10:10 v/v/v/v) (Kristiansen, 1984). Dried plates were sprayed with DMACA reagent diluted 20-fold in methanol and analyzed for flavan-3-ols. PA samples were depolymerised and converted to anthocyanidins by acid hydrolysis and then analysed as for anthocyanin samples.

### Measurement of Anthocyanin and PA Content

PA monomers and polymer were quantitated using DMACA reagent in a 96 well plate reader (Molecular Devices, Spectra MAX 340 PC). Standard curves were prepared by serial dilution of catechin monomer, trimer and condensed tannin (isolated from O. viciifolia and quantitated by weight) standards (Tanner et al., 1994). The plate was scanned between 600 and 700 nm for a peak at 640 nm within 15 minutes of the addition of DMACA reagent. Samples containing PA showed a precipitate after 2 to 3 hours, whereas small polymer standards did not. This method was also used to detect PA contamination of anthocyanin preparations.

Leaf anthocyanin extracts were scanned from 410 to 600 nm to determine the anthocyanin absorbance peak at about 520-530 nm. It was found that mutants such as tt3 gave a broad peak between 510 to 530 nm, even though they lack anthocyanin. For the purposes of calculation, the OD at 600 nm was subtracted from the peak anthocyanin absorbance value. Seed anthocyanin concentrations were calculated using OD εmax-OD 600 nm gm⁻¹ fresh weight of material. Anthocyanin extracts were analysed similarly, with anthocyanin being expressed for both leaf and seed extracts as a % relative to wild type levels, because this value was constant for replicate experiments performed at different times. Ws-2 and Col-7 wild types were found to have different εmax values (scans not shown) and so each mutant was compared to its wild type.

An aliquot of the ethyl acetate extract was dried by vacuum centrifugation at room temperature. The residue was dissolved in 100 μl of water and analysed by HPLC on an Activon (Australia) Goldpack 3 cm×0.46 cm (ID) column packed with 3μ Exsil 100A, ODS C18 packing and eluted at 2 ml/min with a gradient from solvent A (2% v/v aqueous acetic acid) to 60% solvent B (methanol) over 10 min, and returning to starting conditions over 5 min, with the detector set at 280 nm. The void volume of the column and system was 500 μL. Peaks of interest from wild-type Ws-2 seeds were re-purified as described above but using water as solvent A, and the mass determined using HPLC mass spectrometry.

### Quantitation of Monomer and Polymer During Seed Maturation

PA extractions and quantitation were performed as described above. Approximately 50 to 200 mg wet weight of tissue was ground in liquid N2 using a mortar and pestle for extraction. After extraction in 70% (v/v) acetone and removal of the supernatant, the cell debris was dried in a 70° C. oven until a constant dry weight was obtained on two consecutive days. Duplicate samples were kept at −20° C. in residual 70% acetone, and later used for phloroglucinol analysis of the acetone insoluble fraction. The acetone fraction of PA extracts was treated with ethyl acetate to partition the monomers (and some dinners) into the ethyl acetate phase from PA polymers (trimers and longer) that remain in the aqueous phase (Nonaka et al., 1983). Authentic standards were obtained from commercial sources.

### Microscopy

Fresh siliques were harvested and placed directly into DMACA reagent for 16 to 18 hours, rinsed three times with 70% ethanol and then photographed at 6.3× magnification. Mature dry seed was stained similarly for 7 to 10 days until all seed were stained in wild type samples. Samples for sectioning were fixed in glutaraldehyde, treated with OsO₄ (Nielson and Griffith, 1978), lightly counterstained with toluidine blue, dehydrated, embedded and then cut in 0.5 and 1 uM sections. Images of sections at 20× and 63× magnification were obtained with or without a Nomarsky filter. For transmission electron microscopy, developing siliques were harvested and fixed in 100 mM sodium phosphate buffer pH 7.0 (POa) containing 3% glutaraldehyde, treated with 2% osmium tetroxide in PO₄ as described by Abrahams et al., 2002.

### Analysis of Gene Expression.

Siliques for analysis were measured in length to estimate the stage of maturity and then checked by microscopic observation, either in sections or by dissection. For analysis of the expression of TDS6 mRNA, tt2, #8-5 (Abrahams et al., 2002), Col7 and tds6-1 were grown and leaves, stems, flower buds and 4-5 mm length siliques collected in liquid N2 and stored at −80° C. until use. For the extraction of RNA, monomer and PA from aging siliques, dehiscence was when the youngest silique opened if the end was touched gently. The stages 1 to 10 are pools of three siliques from dehiscence (10), upwards on the stem. Siliques from stages 1 to 10 were dissected to determine the stage of embryo development.

Leaf, stem, flower bud and developing silique material was harvested from Col7 plants for analysis of gene expression using RT-PCR. RNA was isolated using the SV Total RNA isolation System (Promega), with 1% (w/v) polyvinylpyrrolidone being added to the extraction buffer to prevent PA (in seeds) from binding nucleic acids. Two micrograms of RNA was reverse transcribed using Thermoscript RNase H-Reverse Transcriptase (Invitrogen Life Technologies), PCR amplified for 15 cycles using Klentaq polymerase (Clontech). The primers used for TDS6 were designed to amplify across intron 4: primers CFI-for (5′-CCAGTCCAAGTACTTCAAAGCTAACTCCG-3′) and TDS6-3′ (5′-TCCCCCGGGATGGTTCTTAGGTTAAAACTGCGGAG-3′), giving products of 624 bp from cDNA and 1179 bp from genomic DNA templates; for TDS2 expression, the primers were designed to amplify across intron 2 of the TDS2 gene: TDS2-for (5′-CGTCACAGACTCCAATCTTACCGTCC-3′) and TDS2-3′ (5′-CCGAGCTCCCATGCTGTTACTTGGTTTAGTTC-3′), giving products of 291 bp from cDNA and 372 bp from genomic DNA templates; white for the TT12 gene the primers spanned introns 2, 3 and 4: primers TT12-for (5′CGTTCCTCTACTGGTACTCGGGTCC-3′) and TT12-rev (5′-CACAAGCACGATGACACAGAGAAC-3′), giving products of 684 bp from cDNA and 922 bp from genomic DNA. The primers used could therefore discriminate between products amplified from cDNA and residual genomic DNA in the RNA samples in the RT-PCR analysis. The number of rounds of amplification was limited to fifteen, based on optimised conditions. Primer annealing temperatures were optimised using a gradient PCR block (Hybaid). The histone H2A primers were from Devic et al. (1999). Products were separated on a 2% (w/v) agarose gel, the gel blotted onto N+ membrane (Hybond) using 0.4 M NaOH, and probed with DNA fragments of TDS6, TT12, TDS2 and H2A amplified from genomic DNA, sequenced to confirm their identity.

### GFP Localisation

The Green. Fluorescent Protein (GFP) gene was obtained from Dr Jen Sheen, Department of Molecular Biology—Massachusetts General Hospital, its DNA sequence having been modified for optimal expression in plants. The coding region of 7DS2 was amplified by PCR using the primers TDS2-ATG-EcoRI (5′-GGAATTCGTGAGTAAGGAAGAAATAATCAGGAAC-3′) and TDS2-w/o stop HindIII (5′-CCCAAGCTTTAGACCCTTGGAGCCAGGGAGGTC-3′), and inserted into the vector pART7 (Gleave, 1992). Fusions of TDS2:GFP were made by cloning the TDS2 gene, 5′ to and in-frame with GFP in the pART7 vector. The 35S-TDS2:GFP-OCS expression cassette was then sub-cloned into the binary vector pART27 (Gleave, 1992), and transformed into Col-7 and tds2. Transgenic plants were selected by germinating seed on MS media containing hygromycin (10 mg/L)

The coding region of TDS6 was amplified by PCR using the primers TDS6-ATG EcoRI (5′-CGGGATCCAGAGATGGTCATGGTTCACGAGG-3′) and TDS6-w/o stop HindIII (5′-CCCAAGCTTGGTTAAAACTGCGGAGATTG-3′), and inserted into the vector pART7 (Gleave, 1992). Fusions of TDS6:GFP were made by cloning the TDS6 gene, 5′ to and in-frame with GFP in the pART7 vector. The 35S-TDS6:GFP-OCS expression cassette was then sub-cloned into the binary vector pART27 (Gleave, 1992), and transformed into Col-7 and tds6-1. Transgenic plants were selected by germinating seed on MS media containing kanamycin (50 mg/L).

### Confocal Microscopy

For visualisation of GFP, tissue samples were mounted on slides for observation using a Lelca SP2 confocal microscope. The sample was excited at 458 nm, and the emitted fluorescence from 465 to 525 nm was collected. Autofluorescence was monitored by collecting emissions from 600 to 720 nm. Under these conditions fluorescence from chloroplasts in leaves was minimal.

### Example 2

### Isolation of PA Synthesis Mutants

To identify the steps involved in PA biosynthesis, we screened T-DNA tagged mutants available from seed stock centres using the DMACA stain as described above to detect seeds with altered PA synthesis or accumulation. We reasoned that mutants specific for the PA pathway should have normal anthocyanin but altered PA content. After identifying pools containing mutant seed, individual plants were grown from duplicate unstained seed pools, their seeds were collected and stained with DMACA. Ten individual mutants with either reduced PA or an altered pattern of accumulation of PA were identified from the screen. Allelic complementation tests were done by crossing the mutant plants to determine the number of loci represented by the mutants. The results of the complementation analysis and a summary of the mutant phenotypes appear in Table 1. The ten mutants fell into eight complementation groups, representing a mutation frequency of PA-free mutants of at least 1 in 1900 mutants screened. The frequency of anthocyanin positive PA-free mutants was 1 in 2700. Not all of the mutants initially observed in the pools of seed were actually Isolated since some were represented by only 1-2 seed in the initial stained pool that may have failed to germinate or grow to maturity in the duplicate pool, tds1, tds2 and tds3-1 were from the Feldmann collection of mutants in the Ws-2 background, tds3-2 and tds4 were from INRA in the Ws-4 background, and tds5, tds6, tt7-3, tt8-4 and tt8-5 were from the Weigel mutant collection in the Col-7 background.

One of the mutants shared the same phenotype as tt7-1, which is mutated in the F3′H gene (Schoenbohm et al., 2000). Allelism tests confirmed that this mutant was an allele at the 777 locus and it was named tt7-3. Similarly, because of their phenotype, two of the mutants were crossed to tt8-1, and found to be alleles at the TT8 locus and were named tt8-4 and U8-5. The mutants ban (Devic et al., 1999), tt1 and tt2 (Shirley et al., 1995) were specifically of interest because of their potential role in PA biosynthesis, and crosses were performed between ban (F36), tt1-1 and tt2-1 and each new mutant to test for possible allelism. None of these new mutants was allelic to ban, tt1-1 or tt2-1 and so they were named tds for tannin deficient seed.

Reciprocal crosses between the mutants and wild-type plants revealed that all of the F₁ testa exhibited phenotypes conferred by the maternal parent. All F₂ seed displayed a wild-type phenotype. Segregation of the mutant phenotype was observed in F3 seed. These results were consistent with gene expression in maternal tissue and the inheritance of the PA-free phenotype as a recessive trait. The segregation of the mutant and wild-type phenotypes in F₃ seed after crossing to wild type was determined. The segregation of marker genes (kanamycin or herbicide resistance) in the F3 generation was also assessed, independently of the mutant phenotype. Three of the mutant phenotypes, tds4, tt7-3, and tds5, were shown to segregate independently of the resistance markers, indicating that they resulted from a spontaneous mutation or partial T-DNA insertion. The DNA flanking the T-DNA in the tt8-4 mutant allele was obtained by plasmid rescue and the interrupted gene encoded the bHLH protein previously described by Nesi et al., (2000).

Description of the tds Mutant Phenotypes with DMACA Staining

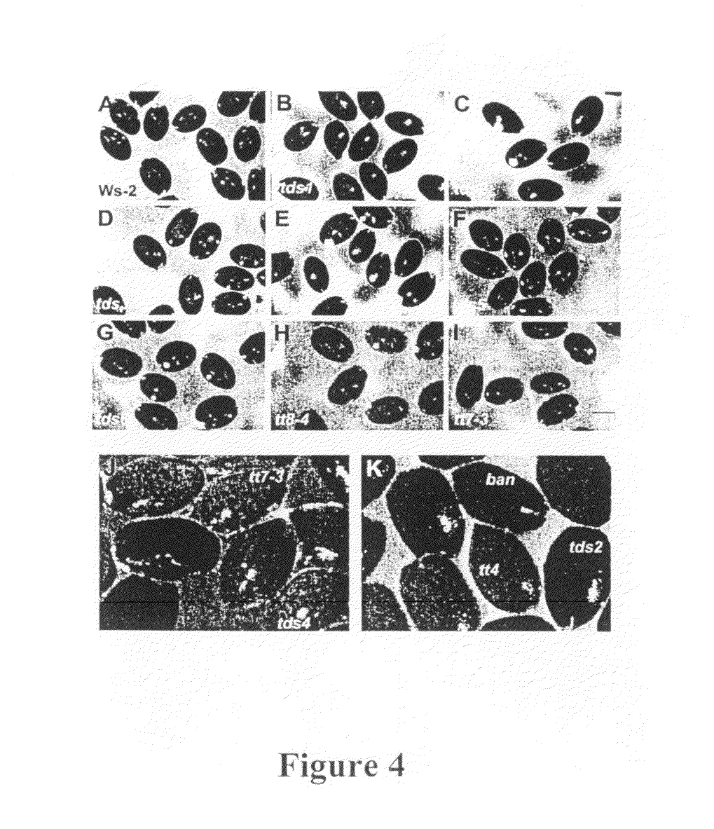

Unstained seed of mutants tds1, tds2, tds3-1, tds5 and tds6 were tan in colour, slightly paler than wild type seed grown under the same conditions whereas tds4 seed were pale yellow in colour. The colour of seed changed with time after harvesting, due to oxidative processes in the seed, in a similar way to that described previously for some tt mutants (Debeaujon et al., 2001). FIG. 4 shows the phenotype of mature seed of the mutants stained with DMACA. Mutants tds1, tds2 and tds5 were DMACA negative apart from a small area at the basal end of the seed (FIG. 4B, C, F) which appeared to accumulate PA. tds3-1 was uniformly DMACA negative, including the basal end of the seed (FIG. 4D). The tds6 mutant had a slight DMACA reaction (FIG. 46), although clearly distinguishable from wild-type (FIG. 4A). Mutants tds4 and tt7-3 (FIG. 4E, I) both showed an altered pattern of PA deposition, tt7-3 showing solid spots, whereas tds4 had a more patchy staining pattern with DMACA. An enlarged image of tds4 and tt7-3 DMACA-stained seed is also shown (FIG. 4J). In addition to the spots of PA, tt7-3 also had spots of anthocyanin. We also observed this spotty pattern of PA and anthocyanin accumulation for tt7-1 in the Landsberg ecotype (Koomneef et al., 1982). tt8-4 was also yellow when not stained and was uniformly DMACA negative (FIG. 4H) including the cells of the basal end of the seed. Mature tt4 and ban seed were also stained and found to be DMACA negative (FIG. 4K), shown in a mixed pool with wild type and tds2 for comparison.

Since only the seed coat produces PA in Arabidopsis, we were able to harvest whole siliques to investigate the accumulation of PA in seeds. Siliques from all stages of development were removed from plants and placed directly into DMACA stain to visualise DMACA reacting PA and precursors of PA. In contrast to the DMACA reaction of mature seed, the developing seed from all the mutants except tt8-4 stained positively with DMACA. Some differences in the intensity of staining were observed, both between mutants and compared with wild type. The mutants tt7-3 and tds4 showed the isolated patches of DMACA staining, observed in mature seeds, throughout their development.

### Qualitative and Quantitative Assessment of Anthocyanin

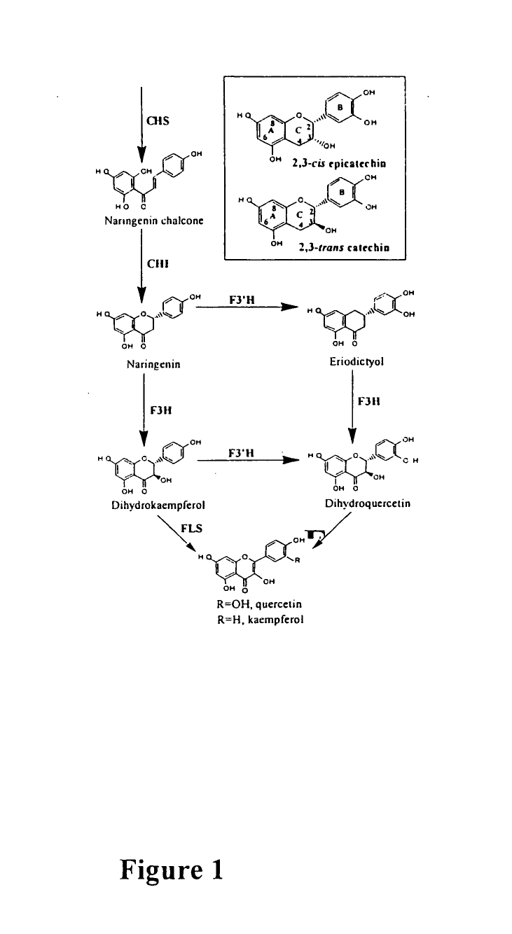

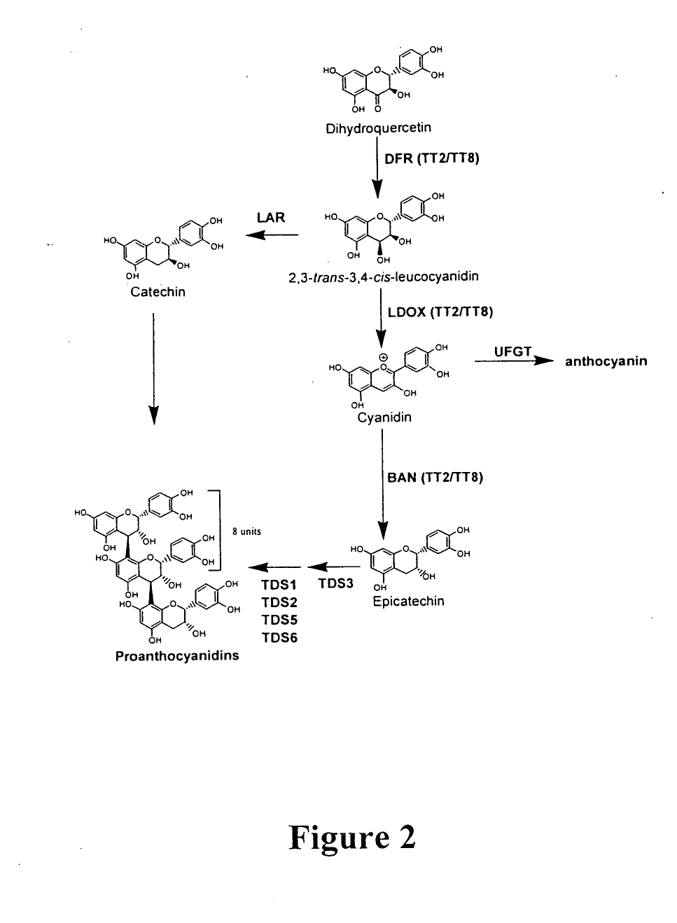

Since anthocyanin and PA synthesis share the same sequence of reactions to the common intermediate leucocyanidin (FIGS. 1 and 2), it was important to determine if the mutants were specific to the PA branch of the pathway. Mutants of specific interest would be expected to be anthocyanin positive and PA-free. For this reason, analyses of tt7-3, ttd-4, U3-1 and ban (F36) mutants were included in this study as a reference point for the identification of the tds mutant phenotype (Koomneef, 1990; Shirley et al., 1992; Albert et al., 1997; Devic et al., 1999; Nesi et al., 2000; Schoenbohm et al., 2000).

Thin-layer chromatography (TLC) was a useful way of comparing whole seed and leaf extracts to visualise flavonols and anthocyanins in the same sample. Although both flavonols and anthocyanins were easily visualised on TLC plates, the NP (Natural Products) stain reagent differentially stained flavonols. Flavonols that contain two adjacent hydroxyl groups on the B-ring (e.g. quercetin) stained orange while those having one hydroxyl group on the B-ring (e.g. kaempferol) stained yellow (Wagner, 1984). The NP stain also enhanced the appearance of anthocyanins, which were present at lower concentrations than the flavonols in the tissues analysed. Since flavonols and anthocyanins may be present in tissues as glycosylated, acylated or other derivatives, it was useful to acid hydrolyse extracts to convert the modified intermediates to their common aglycone form (as shown in FIG. 2).

The flavonols kaempferol and quercetin were present in the seeds of most mutants, including tt3 (which lacks DFR) and ban. The exceptions were tt4, which lacked chalcone synthase and would not be expected to contain these intermediates, and tt7-3, which accumulated only the monohydroxylated kaempferol. The large amount of flavonols present in the seed extract prevented detection of anthocyanins in these samples. Leaf anthocyanin extracts contained only kaempferol and not quercetin, indicating a difference in the expression of F3′H or flavonol synthase genes between leaves and seeds of Arabidopsis. Cyanidin was clearly visible on the TLC of acid hydrolysed leaf anthocyanin extracts. Analysis using TLC showed that most of the mutants accumulated wild type levels of the flavonols kaempferol and quercetin and were therefore mutated in genes acting at or beyond DFR in the pathway.

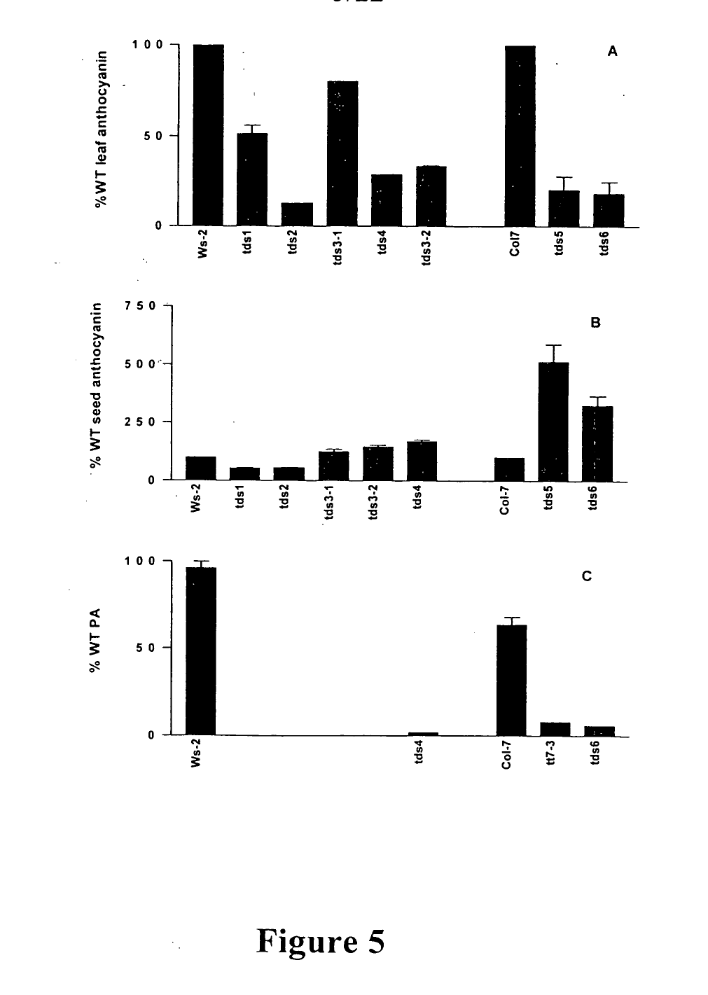

The visible spectra of anthocyanins extracted from leaves and mature seeds were used to determine quantitative and qualitative differences in anthocyanin accumulation. All of the mutants produced 20-80% of wild-type anthocyanin in leaf material, as shown in FIG. 5A. Two of the mutants, tds1 and tds2, showed a decrease in concentration of anthocyanin in mature seeds (FIG. 5B), but all other mutants accumulated 20-500% more anthocyanin than wild type in seeds (FIG. 5B). Leaves produced more anthocyanin than seeds, per gram fresh weight of material, conversely the seeds tended to accumulate larger amounts of flavonols.

### Example 3

### Analysis of PA and its Intermediates In Mutant Plants

Since developing seed of wild-type and tds mutants possessed DMACA-reacting compounds and the mature seed of tds1, tds2, tds3-1 and tds5 did not (FIG. 4), PA was extracted from developing siliques and mature seed and analysed using TLC and HPLC to identify the DMACA reacting compounds. Measurable amounts of PA were extracted from mature seed of tds4, tds6 and tt7-3, but reduced compared to wild-type (FIG. 5C). Mature seed of the other mutants did not accumulate measurable amounts of PA. Col-7 accumulated 30% less of the PA than Ws-2 wild-type mature seed when grown under the same conditions. The two mutants tds4 and f/7-3 that showed a patchy or spotted pattern of PA accumulation also showed a marked decrease in extractable PA, 2 and 8% that of their wild type, respectively. The mutant tds6, which was slightly positive when stained with DMACA, accumulated 6% of the wild-type level of PA in the mature seed. Acid hydrolysis of Ws-2 PA produced cyanidin only.

Extracts were also prepared from developing Arabidopsis siliques, which included seeds up to the late heart stage or walking stick stage of embryo development. The same extract was used to analyse both PA precursors and PA polymer. PA polymer was not detected in the developing seeds of any sample, including wild type. The fraction containing PA precursors was separated using TLC and sprayed with DMACA reagent. A compound was detected in tds1, tds2, tds3-1, tds4, wild type, tds5 and tds6 mutants that was absent from tt8-4 and tt3 control extracts. This compound had the same RF as authentic epicatechin. Since tt3 and tt8-4, both of which lack DFR activity, did not accumulate this intermediate, it might be related to steps downstream of DFR in the PA synthesis pathway (FIG. 2). Extracts from tt7-3 contained a faster migrating compound to that of wild type, which may correspond to afzelechin or epiafzelechin, however standards of these compounds are not available for identification of these intermediates by comparison of RF values. In this solvent the monohydroxylated flavonoid isomers migrate faster than the corresponding dihydroxyiated isomers consistent with our tentative identification of this compound as afzelechin or epiafzelechin.

The extracts containing PA precursors were also analysed using HPLC. A peak with retention time of 4 min was observed in tds1, tds2, tds3-1, Ws-2, tds5 and tds6 samples. Extracts of tt3 and ban did not have this compound, so it might be a product of enzymatic steps beyond DFR and BAN in the pathway. This compound was purified by preparative HPLC methods and found to be DMACA positive and comigrated with the single DMACA-reacting bands in samples except tt7-3 on the TLC. HPLC mass spectrometry was used to determine the compounds molecular mass as 291.2 Daltons, which was the same as both protonated catechin and epicatechin. Because this compound co-migrated with standard epicatechin on both TLC and HPLC, had the same molecular mass as epicatechin, and like epicatechin reacted strongly with DMACA yielding a blue product, it was likely to be epicatechin. In addition to this peak, other mutants produced novel intermediates, or enhanced amounts of intermediates relative to wild-type. The mutant tds4 produced a unique compound with a retention time of 2.92 min and tds4, (ds5 and tds6 produced an unidentified compound with a retention time of 5.3 min, approximately 2-4 fold greater than wild-type.

Microscopic Examination of the Localization of PA in Wild Type, tds4 and tt7-3

When whole wild-type Arabidopsis seedlings were stained with DMACA, only the developing seed coat gave a positive reaction for PA. Sections of developing seeds were treated with OsO₄ to detect the accumulation of PA or its precursors, and then lightly counterstained with toluidine blue to show cell structure in the tissue. The PA was synthesised in wild-type Arabidopsis in the endothelial layer of the testa. PA, or its precursors, was visible as a grey or black deposit in endothelial cells from as early as the two-terminal cell stage of embryo development, which coincided with 18 hours after flowering (Mansfield, 1994). PA appeared in all cells of this layer. In wild-type seeds, the vacuole containing PA occupied almost the entire cell contents, giving the general appearance of containing PA uniformly throughout the endothelial layer of the seed coat. In tt7-3, the PA appeared as discrete spots within cells, reminiscent of the pattern seen with DMACA stain at lower magnification in whole seeds. Higher magnification showed this staining to occur in the vacuole of the cell. PA was visible in tds4 later in development than for wild-type and was associated with small provacuolar bodies that did not appear to fuse with each other or the main vacuole. Generally, more mature seeds were difficult to section due to the treatment with osmium tetroxide.

### Example 4

### Cloning and Structural Analysis of the TDS6 Gene

Recovery of the T-DNA Tagged Sequences in Mutant Line tds6-1

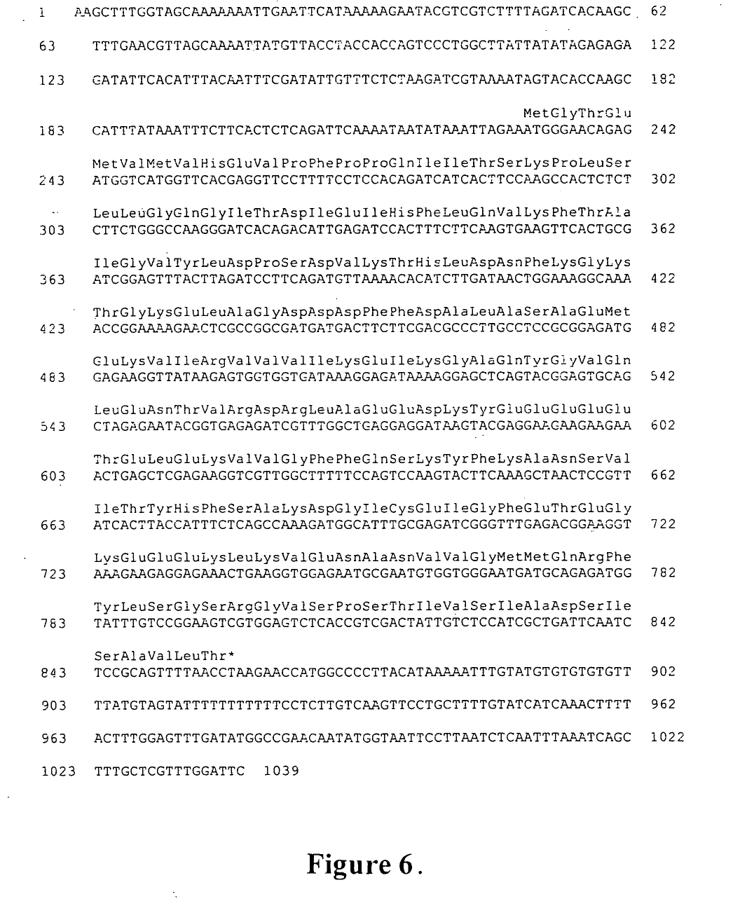

The tds6-1 tagged line was created by insertion of a T-DNA from the vector pSKI015 (Weigel et al., 2000), encoding the bar gene that confers resistance to phosphinothricin into Col7 wild type plants (Weigel lab home page http://www.salk.edu). and was isolated from pool 21292 from the Weigel collection (Abrahams et al., 2002). Genomic DNA was isolated from leaf material of the mutant plant using Plant DNAzol Reagent (Invitrogen Life technologies) and cut with a number of restriction enzymes. When probed with a PCR amplified DNA fragment for the bar gene, at least six hybridising bands were observed, an indication of multiple T-DNA insertion sites. The homozygous recessive tds6-1 plant was crossed with wild-type Col7 plants, DNA was isolated from leaves of F2 plants, and then analysed by Southern blot hybridisation. The F₃ seed from the plants were stained with DMACA to determine seed PA phenotype. One of the resulting tds6 mutant progeny was found to have fewer T-DNA insertions than the tds6-1 parent, and was therefore used for plasmid rescue of the DNA flanking the T-DNA insertions. 20 μg of DNA was cut with BamHI in 50 μl volume, desalted using an S200 spin column (Pharmacia), self ligated in a 60 μL volume and then transformed into TOP10 cells (Invitrogen) [mcrAΔ(mrr-hsdRMS-mcrBC)]. Recovered plasmids were sequenced, and one was found to represent a T-DNA inserted 50 basepairs (bp) upstream of the predicted translation start codon of the gene, At5g05270, the nucleotide sequence of which is shown in FIG. 6.

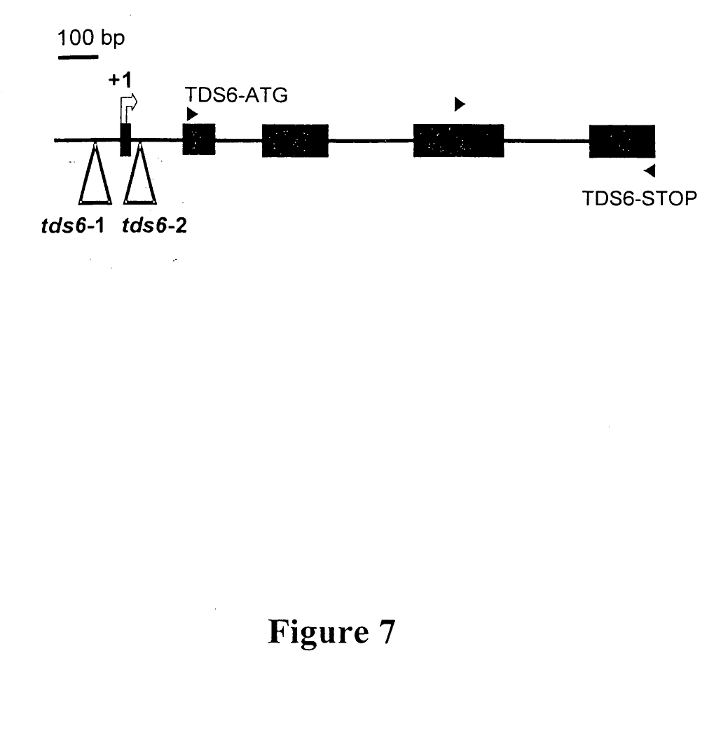

The tds6-2 allele was identified from a search of the databases of T-DNA insertion lines available from the SALK Institute (SALK_{—}096551). The T-DNA was inserted within the first exon of the At5g05270gene (shown schematically in FIG. 7), hereinafter called the TDS6 gene. This line was used to confirm that insertion of a T-DNA into the TDS6 gene caused the mutant phenotype observed in tds6-1.

When the TDS6 gene sequence was initially identified, the gene annotation in the TAIR (The Arabidopsis Information Resource) database suggested that the TDS6 gene consisted of four exons and three introns. Updates of the databases, that Included cDNA sequences, suggested that the TDS6 gene consisted of five exons and four introns, extending the length of the gene in the 5′ direction. This is the gene structure shown in FIG. 7. The PCR primer TDS6-ATG was designed to amplify the 7DS6 gene according to the initial annotation on the database, so the TDS6 protein that complemented the tds6-1 mutation was five amino acids shorter at the N-terminus than the protein predicted from cDNA sequences. In addition to the cDNA sequences available from the database, we amplified TDS6 cDNAs from developing Col7 silique RNA. DNA sequence analysis showed that all cDNAs were identical to those found in the EST database, and were consistent with the gene structure shown in FIG. 7.

### Example 5

### Molecular Complementation of the tds6 Mutation

In order to test for complementation of the PA-deficient mutation in tds6 mutants and thereby confirm the role of TDS6 in PA biosynthesis, a 35S:TDS6 construct was made and expressed in the tds6-1 mutant background. The 35S-TDS6 construct, which lacked the five N-terminal amino acids mentioned above, was made by PCR amplification of the coding region of the TDS6 gene from Col7 genomic DNA using primers TDS6-ATG (5′-CGGGATCCCAGAGATGGTCATGGTTCACGAGG-3′) and TDS6-3′ (5′-TCCCCCGGGATGGTTCTTAGGTTAAAACTGCGGAG-3′). The coding region was inserted into the binary vector pBI121 (Clontech) in the correct orientation relative to the CaMV 35S promoter, replacing the GUS gene. The vector pBI121 carried an Nptll gene that conferred kanamycin resistance. After confirmation of the sequence, the 35S-TDS6 construct was transformed into the tds6-1 mutant background using a modified vacuum infiltration method with Agrobacterium (Bechtold et al., 1993), and the resulting T₁ seed germinated on MS medium containing phosphinothricin (10 mg/ml) and kanamycin (50 mg/ml) to select for transformed plants. T₁ plants were grown, T₂ seed collected and stained using 1.0% (w/v) DMACA. DNA was Isolated from T₁ plants and analysed by Southern blot hybridisation for the presence of bar and Nptll genes, present in the first (mutating) and second (complementing) T-DNAs, respectively.

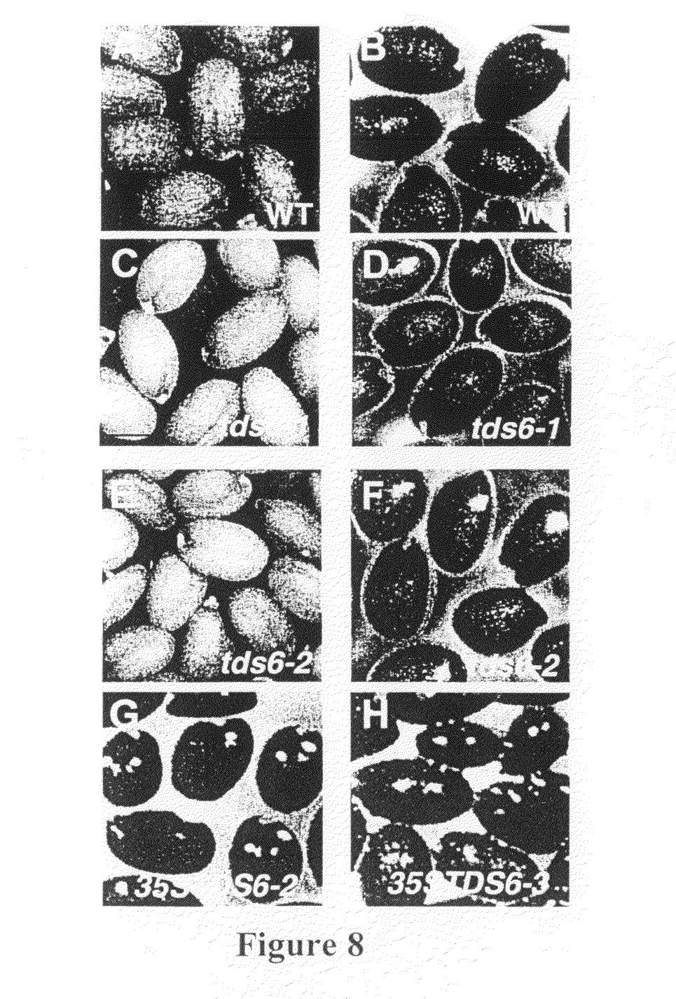

When comparing seedcoats without DMACA staining, tds6-1 and tds6-2 mutant seed were slightly paler than wild-type due to the presence of PA in the wild-type. When stained with DMACA, seeds of tds6-1 and tds6-2 were very similar in appearance, but paler than wild-type seed. Because the seed coat was maternal tissue, we expected to observe delayed inheritance of the transgene in the T₂ seed transformed with 35S:TDS6. FIGS. 8G and 8H show pools of DMACA stained T2 seed from two of the complemented tds6-1 progeny, namely 35S:TDS6-2 and 35S:TDS6-3, respectively. Both pools show near wild-type levels of staining with DMACA. Other pools of T₂ seed showed varying degrees of staining with DMACA, presumably due to differing levels of expression of the 3SS:TDS6 gene due to position effects. These data show that mutation of TDS6 caused the PA-deficient phenotype observed in the tds6 mutants and the role of TDS6 in PA synthesis or accumulation.

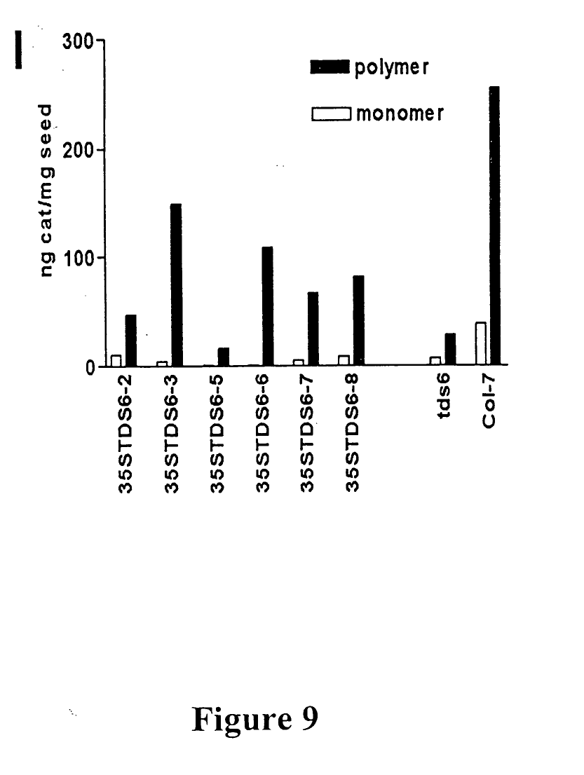

To demonstrate that the level of DMACA staining paralleled the production of PA, both PA precursors (monomers) and PA were extracted from pools of T₂ seed. PA and PA precursors were extracted from approximately 20 mg aliquots of T₂ seed of wild-type, mutant and complemented plants, and quantitated against a catechin standard, using 0.1% (v/v) DMACA reagent diluted in methanol (Abrahams et al., 2002) and a 96 well plate reader. Due to the limited availability of seed from individual plants, samples were not extracted in duplicate. Wild-type produced approximately 255 ng of catechin/mg seed of polymer and 40 ng/mg seed of epicatechin monomer (FIG. 9), whereas tds6-1 produced 30 ng/mg polymer and 5 ng/mg monomer. Different transgenic lines of 35S:TDS6 T2 seed, which reflect the expression of the hemizygous 35S.TDS6 gene, produced between 15 and 150 ng/mg seed polymer (FIG. 9), which could account for the differences in staining observed with DMACA. The measured level of PA in the seeds correlated well with the intensity of staining with DMACA.

T₂ seed from transgenic fine 35S:TDS6-3 in the tds6-1 background, containing the highest level of PA compared with wild-type, was germinated on MS medium without selection and T₃ seed from 52 progeny were analysed using DMACA staining to follow segregation of the phenotypes. DNAs from 24 of the progeny were analysed by Southern blot hybridisation to observe the co-segregation of the T-DNAs and their respective phenotypes. Southern blot analysis of the 35S:TDS6 complemented lines showed that the plants contained both BAR and Nptll genes associated with each of the two T-DNAs present in their genomic DNA. T₂ seed germinated on MS media containing both phosphinothricin and kanamycin, whereas the tds6-1 seed germinated in the presence of phosphinothricin only. T₂ seed from 35S:TDS6-3, producing the highest amount of PA (FIGS. 8 and 9), was germinated on MS media without selection and grown to produce T₃ seed, which was stained with DMACA to observe segregation of complemented WT and tds6-1 phenotypes. From 52 T₂ derived T₃ seed populations, wild-type and tds6-1 seed phenotypes segregated 42:10, which is not significantly different to a 3:1 ratio (P=0.6365). When Southern blot analysis was done using DNA isolated from 24 of the T₂ plants, the wild-type phenotype co-segregated with the 35S:TDS6 transgene. The co-segregation analysis therefore further confirmed that the TDS6 gene was involved in the synthesis of PA.

### Example 6

### Expression of the 7056 Gene

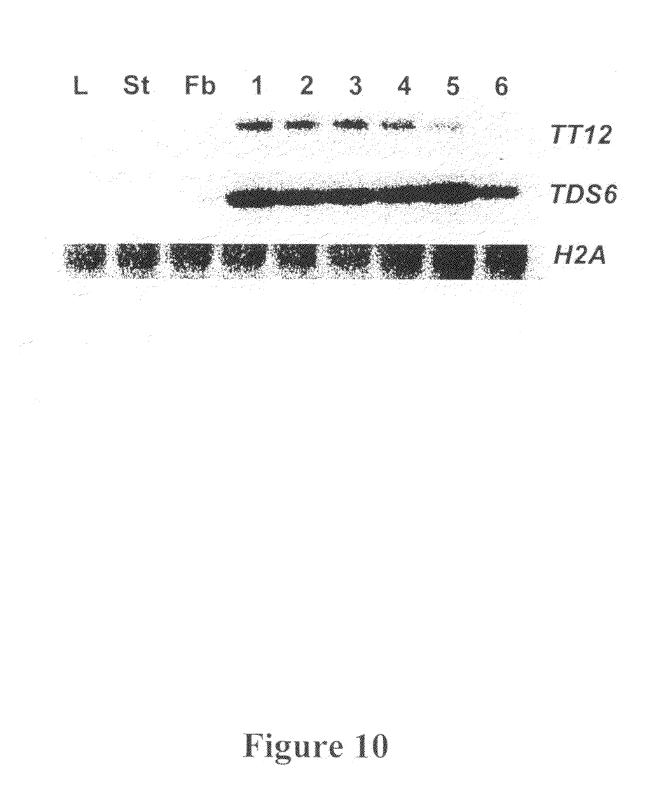

Since PA accumulates only in the endothelial cell layer of seeds of Arabidopsis, we were interested to determine the pattern of expression of the TDS6 gene In Arabidopsis tissues. The expression of the TDS6 mRNA In leaves, stems, flowers and developing siliques was analysed using RT-PCR. TDS6 mRNA was detected in developing siliques, with a trace amount also detected in flowers but not in either leaves or stems (FIG. 10). In comparison, expression of the gene TT12, which encodes a MATE transporter implicated in transport of PA intermediates into the vacuole (Debeaujon et al., 2001), was confined to developing siliques between the two terminal cell and torpedo stages of development, with no expression detected in older samples (FIG. 10). This was the same pattern as previously reported by Debeaujon et al., 2001, indicating that the tissues in the two studies were of similar age and could be compared. TDS6 expression generally coincided with TT12 expression, but continued later into the walking stick or upturned U-stage of embryo development (FIG. 10). The expression of TDS6 mRNA also occurred steadily over a longer period of time than genes such as BAN, the expression of which was limited to early globular stage of embryo development (Devic et al., 1999).

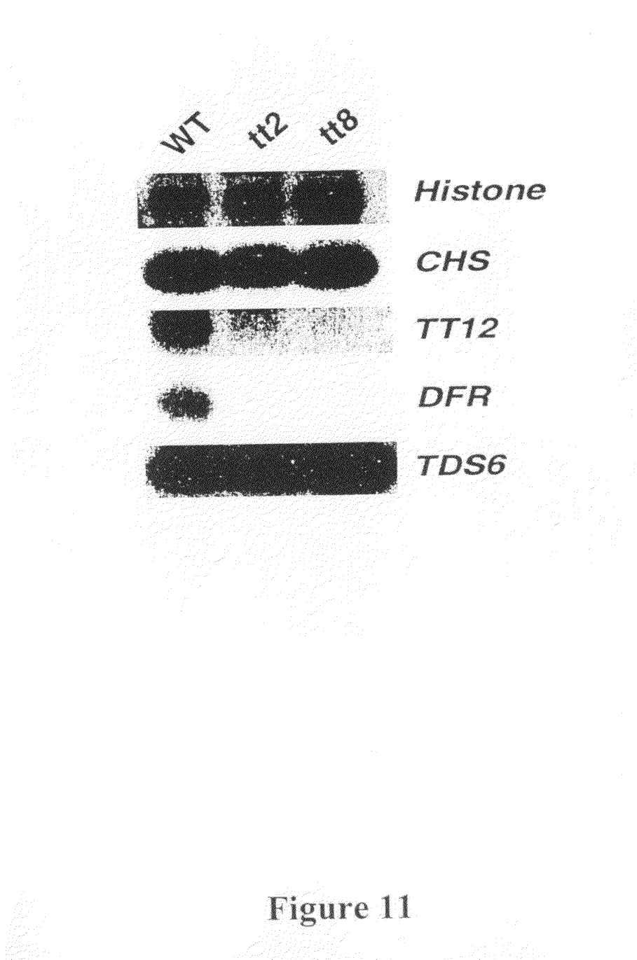

It was known that the regulators, TT2 and TT8, are required for the expression of DFR, LDOX and BAN genes Involved in PA biosynthesis (Nesi et al., 2001) and were involved in the regulation of TT12 expression. The observation that the expression of TT2 occurred up to the torpedo stage of embryo development, whereas TDS6 expression appeared to continue when TT2 mRNA is no longer present, suggested that TDS6 might not be regulated by TT2. To test this, RT-PCR was used to monitor the expression of TDS8 mRNA in tt8 and tt2 mutant backgrounds. FIG. 11 shows expression of CHS, DFR, TT12, TDS6 and histone H2A in wild-type (WT), tt2 and tt8 developing siliques. The CHS gene was included because it is not regulated by TT2 or TT8, as indicated by similar levels of CHS product in WT, tt2 and tt8 samples. In contrast, DFR and TT2 genes were not expressed in tt2 and tt8 mutants, indicating that their expression was regulated by both TT2 and TT8 proteins, as had previously been observed (Nesi et al., 2001). The TDS6 gene was equally expressed in WT, tt2 and tt8 siliques, indicating that TDS6 was not regulated by either TT2 or TT8.

Analysis of the expression patterns of CHS, CHI, LDOX and DFR genes in Arabidopsis seedlings indicated that genes encoding enzyme steps in the anthocyanin pathway could be considered “early” and “late biosynthetic genes” (Kubasek et al., 1992). These terms have also been used to refer to the expression patterns of genes involved in PA biosynthesis in developing seeds. Transcripts for TT2 and so-called “late biosynthetic genes” such as LDOX, BAN and TT12 decreased rapidly from the torpedo stage of embryo development onwards (Nesi et al., 2001). In contrast, the expression of the TDS6 gene occurred throughout seed development, continuing into the walking stick or upturned-U stage of development and then declining steadily until dehiscence. TDS6 is presumably required for PA synthesis after the transport of chain initiation units, extension units, or both has ceased to occur, depending on the turnover rate of the TT12 transporter protein. Therefore, TDS6 might be considered a “very late PA biosynthetic gene”, being involved in an enzymatic step relatively late in the PA biosynthetic pathway.

The finding that TDS6 was not regulated by TT2 or TT8 may suggest that other regulators were required for the control of expression of genes that occur after TT12 including TDS6 and possibly TDS1, TDS2, TDS3 and TDS5, since they are epistatic to BAN. This might explain why, when TT2 is expressed ectopically in Arabidopsis, there was ectopic expression of DFR, LDOX and BAN but not a corresponding appearance of PA (Nesi et al., 2001).

### Example 7

### Predicted Amino Acid Sequence of TDS6 and Homology with Chalcone Isomerase Proteins

Sequence Analysis of TDS6 cDNAs and Predicted Proteins

TDS6-encoding cDNAs were obtained by PCR amplification from developing Col7 silique RNA. PCR products were cloned into pCR2.1 using the Original TA Cloning Kit (Invitrogen), sequenced and analysed using Sequencher software (Gene Codes).

The PSI BLAST program was used to search the database at the National Centre for Biotechnology Information web site http://www.ncbi.nlm.nih.gov/using the predicted protein from At5g02570. EST databases were also searched to find TDS6 homologs. Contigs of overlapping related sequences were created and the identified amino acid sequences were used to perform a ClustalW alignment of the proteins and to create a phylogenetic tree at http://workbench.sdsc.edu site, University of San Diego Supercomputer Centre.

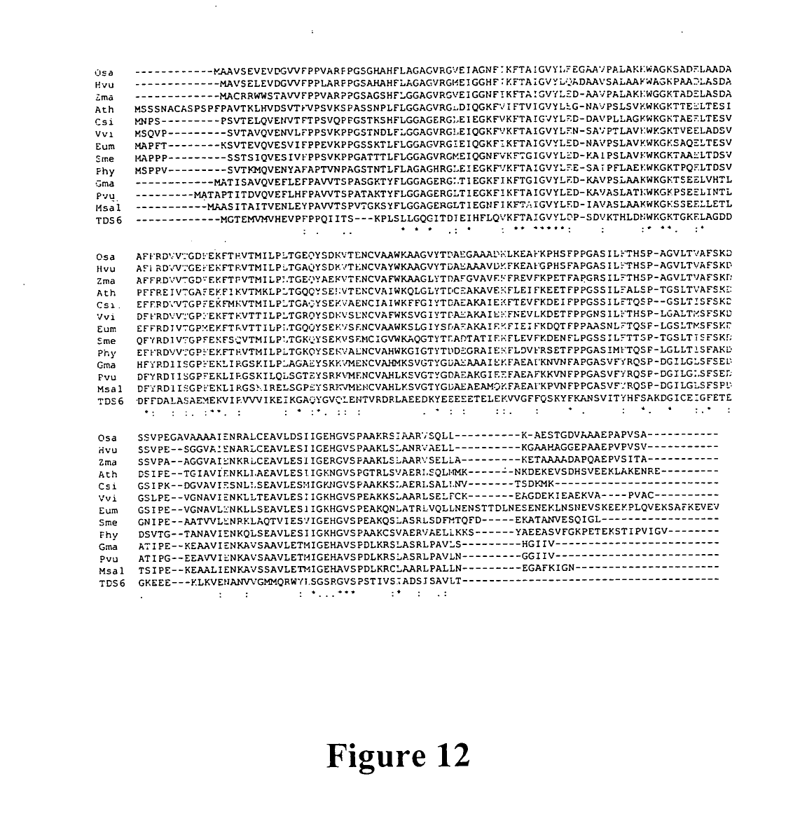

The TDS6 protein sequence predicted from cDNAs encoded a protein of 210 amino acids, shown in FIG. 12 (bottom line). This sequence did not include any apparent signal sequence for trafficking of the protein through the endoplasmic reticulum or for vacuolar localisation.

Homologous TDS6-like proteins were identified in EST databases, including proteins from dicots, legumes and monocots, with 52-70%, 48-62% and 45-60% amino acid identity with Arabidopsis TDS6, respectively. The sequences with the highest similarity to TDS6 were those from Pinus teeda, Gossypium hirsutum and Vitis vinifera, species known to make PA. Others were identified in Glycine max, Medicago truncatula, Triticum aestivum, tomato, Sorghum bicolour, rice, barley, Ipomoea, apricot, apple, Citrus sinensis, and cocao.

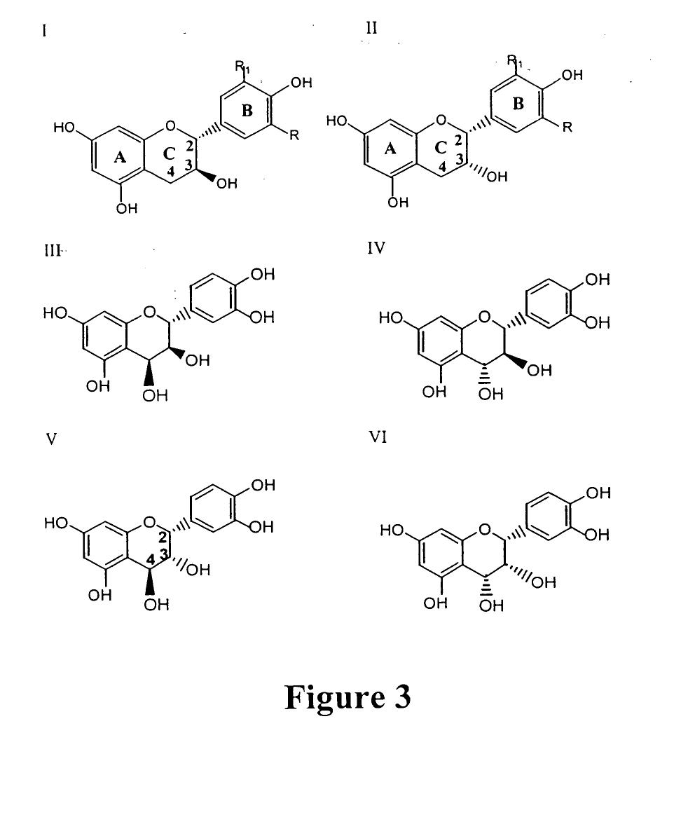

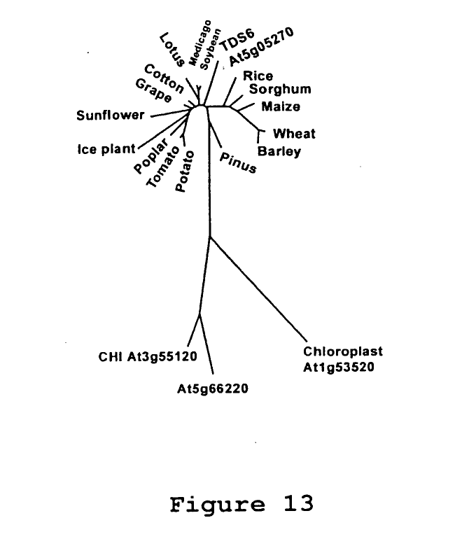

The PSI BLAST search of the NCBI database also identified a number of chalcone isomerase (CHI) sequences that had 27-32% amino acid sequence similarity to the TDS6 predicted protein (see Table 2). TDS6 therefore encodes a CHI-like protein. CHI catalyses the cyclization of naringenin chalcone to naringenin, intermediates common to the anthocyanin and PA synthesis pathways (FIG. 1). The homology of TDS6 with CHI's is spread uniformly throughout the protein. In FIG. 13 the tree produced by ClustalW shows a dumbbell-tike structure, showing that CHI and TDS6 form distinct groups of proteins. The monocot and legume TDS6-like and CHI sequences tend to cluster into distinct groups. TDS6 lacked some of the residues located in the β3a, β3b, α4 and α6 domains associated with the naringenin-binding cleft identified in CHI from Medicago sativa (Jez et al., 2000) (FIG. 3), whilst Thr 190 and Met 191 (or their substitutes Ser and lie in non-legumes), which are suggested to play a role in influencing substrate preference of CHI, are substituted by Tyr and Leu in TDS6. This suggests that TDS6 does not act as a chalcone isomerase in Arabidopsis, but rather catalyses an alternative reaction required in PA biosynthesis.

### Bacterial Expression and Analysis of Enzyme Activity of TDS6

Since the TDS6 protein had about 25% identity to CHI proteins, we tested the ability of TDS6 protein to catalyse the conversion of naringenin chalcone to naringenin. Active CHI proteins from Medicago sativa (Jez et al., 2000) and Pueraria lobata (Terai et al., 1996) had previously been expressed in Escherichia coli and could be used as a positive control. Therefore, a construct was made for the expression of Arabidopsis TDS6 in coli, containing a histidine tag to purify the expressed protein. TDS6 cDNA was amplified from mRNA of Col7 developing siliques, cloned into pQE32 expression vector (Qiagen), the TDS6 sequence confirmed, and the plasmid transformed into the Escherichia coli M15 expression host. The Medicago sativa CHI cDNA was obtained from Joseph Noel (Structural Biology Laboratory, SALK). Aliquots from 18 hour cultures grown at 30° C. were used to inoculate 25 ml of LB media in a 250 ml baffled flasks, grown at 15° C. for 8 hours, induced with IPTG and then incubated for a further 24 hours at 15° C. The cell pellets were resuspended in 10 mM Tris pH 8.0, 1 mM EDTA, 0.1% (v/v) Triton X-100 and 1 M NaCl and then frozen and thawed three times to lyse the cells. The soluble fraction obtained after 30 minutes centrifugation at 10,000 rpm was used in assays for CHI activity. SDS-PAGE of the coli extract showed the 23 kDa TDS6 and CHI proteins in the soluble fraction of cells grown at 15° C. 0.5 gm of Desmodium uncinatum expanding leaves were ground in liquid nitrogen, and extracted in 1 mL extraction buffer (Tanner et al, 2002). E. coli and Desmodium extracts were desalted using a Nap-5 column (Pharmacia), and used to perform CHI assays according to Lister and Lancaster (1996), using a GBC spectrophotometer and software for kinetic analysis. Naringenin chalcone substrate was obtained from Apin Chemicals, and its purity analysed using Beckman HPLC and System Gold software. Aliquots of 20 to 80 μg of soluble protein were used in each assay, performed in triplicate. coli protein extracts were analysed using SDS-PAGE to determine the solubility and size of TDS6 expressed protein.

When the E. coli cultures were grown at 37° C., the TDS6 protein was present in the insoluble fraction of the cell pellet, however, when the culture was grown at 15° C., soluble TDS6 protein could be recovered. Soluble TDS6 protein was tested for CHI activity using naringenin chalcone as a substrate. The rate of change in absorbance at 380 nm, which is used as measure of CHI activity, was no greater in the presence of TDS6 than background rates of naringenin chalcone spontaneous isomerization. However, when a crude desalted extract from PA-rich Desmodium uncinatum leaves was assayed, the rate of CHI activity was found to be 10 mmol/min/mg protein, indicating that the assay was functioning correctly. We also expressed Arabidopsis CHI in E. coli and showed it to be active in the CHI assay.

Although the TDS56 protein was homologous to CHI, they must perform different roles in the flavonoid pathway. CHI is involved in the cyclisation of naringenin chalcone to form naringenin, early in the flavonoid pathway, whereas TDS6 is involved in PA biosynthesis. The tt5 mutant of CHI was unable to produce flavonols, anthocyanin or PA (Koomneef, 1990), and consistent with this, had much paler seed coat than tds6 seed. On the other hand, the tds6-1 mutant was able to make flavonols, anthocyanin and the PA precursor (monomer) epicatechin. Moreover, it seemed from double mutant analysis that the products of the TDS6 catalysed reaction were required downstream of DFR, LDOX (TDS4), BAN and TDS3, to make PA. The CHI enzyme assays, as well as the flavanol-deficient phenotype of the tt5 mutant indicate that TDS6 was not capable of using naringenin chalcone in vitro, and was therefore unlikely to catalyse the CHI reaction in vivo.

The similarity in the structure of CHI and TDS6 flavonoid substrates could, however, account for the overall sequence similarity between the two enzymes. A number of residues that were highly conserved in CHI's were not conserved in TDS6 or TDS6-like proteins, including Arg36, Arg113 and Thr190. Arg113 and T190 are involved in the hydrogen bonding to the 4′ hydroxyl group of naringenin chalcone, and mutations in these residues lead to fourteen and twenty two fold reductions in Kcat and Kcat/Km values for CHI activity (Jez et al., 2002). Similarly, Arg36 was implicated in catalysis (Jez et al., 2002) and mutations that altered its position in the active site led to changes in kinetic parameters. Other residues involved in the hydrogen bond network located at the bottom of the active site cleft were maintained in TDS6, including T48 and Y106 (Jez et al., 2002).

### Example 8

### Analysis of the PA and Monomer Composition of Wild-Type Arabidopsis

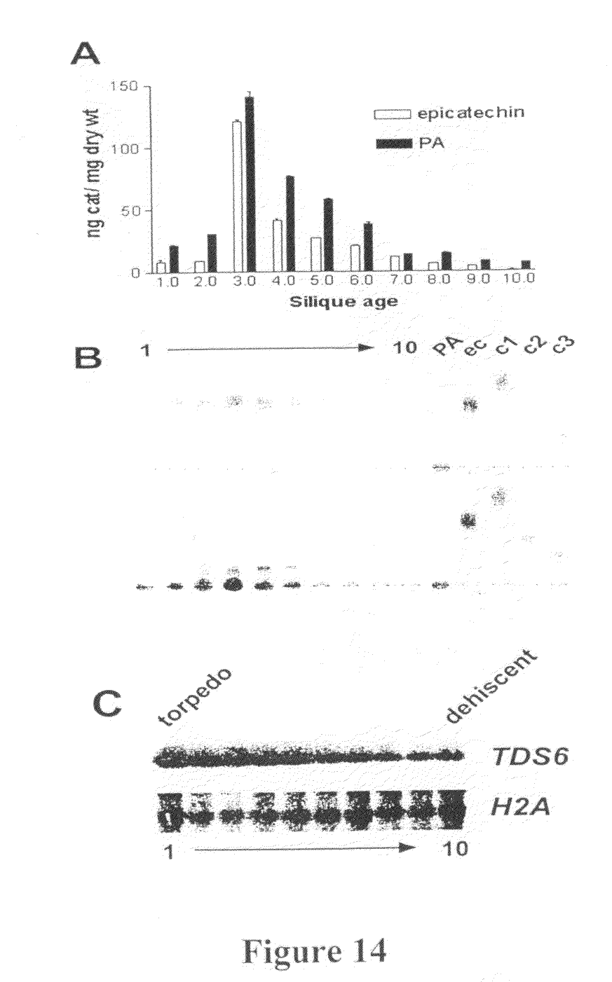

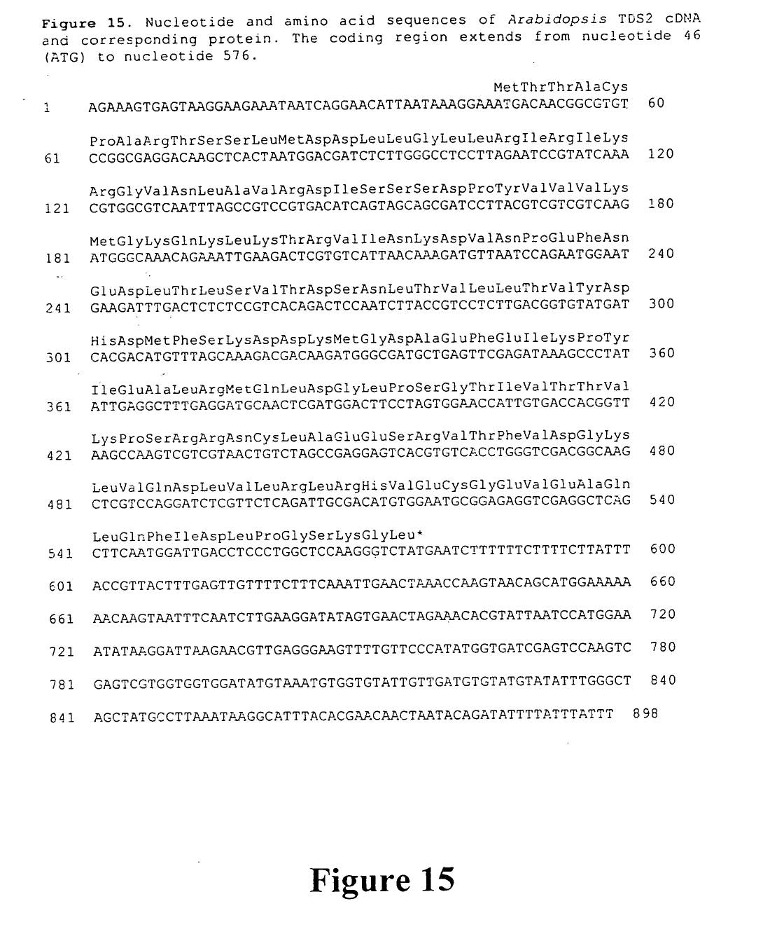

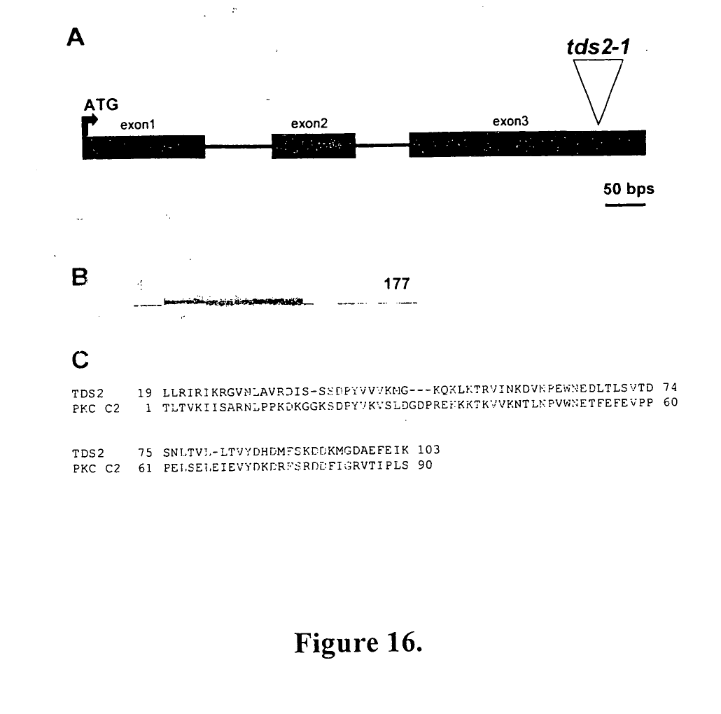

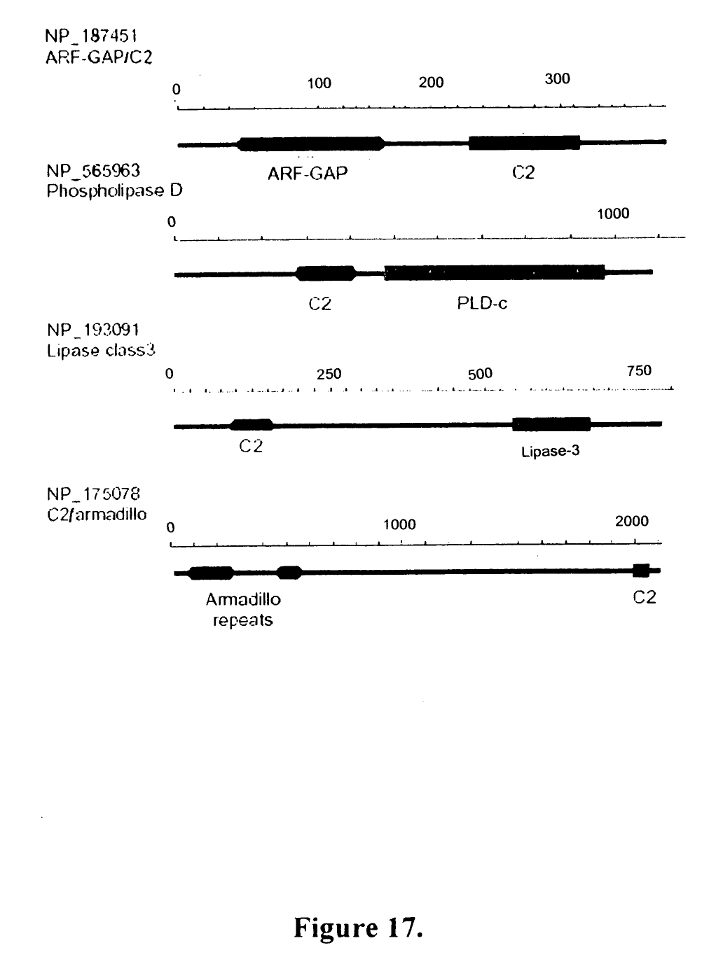

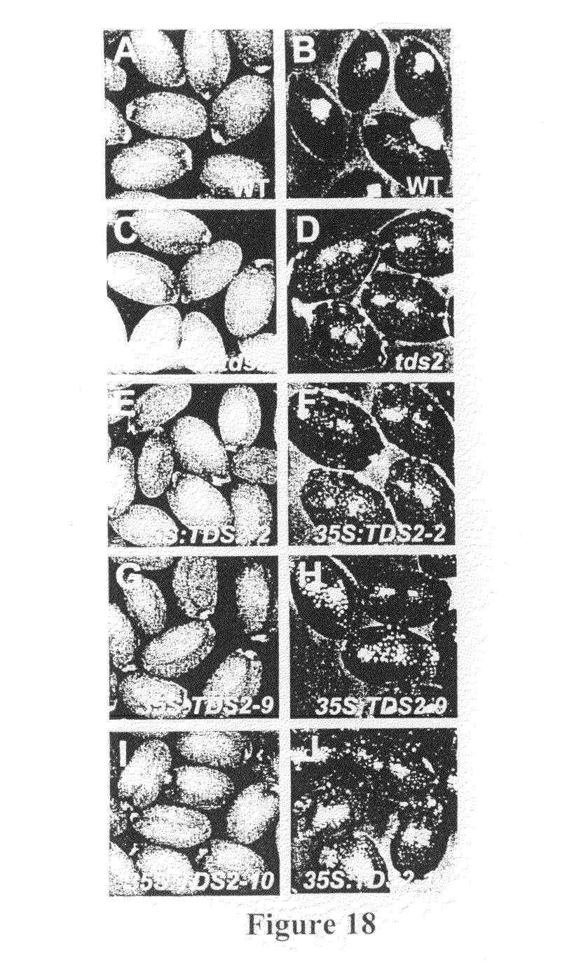

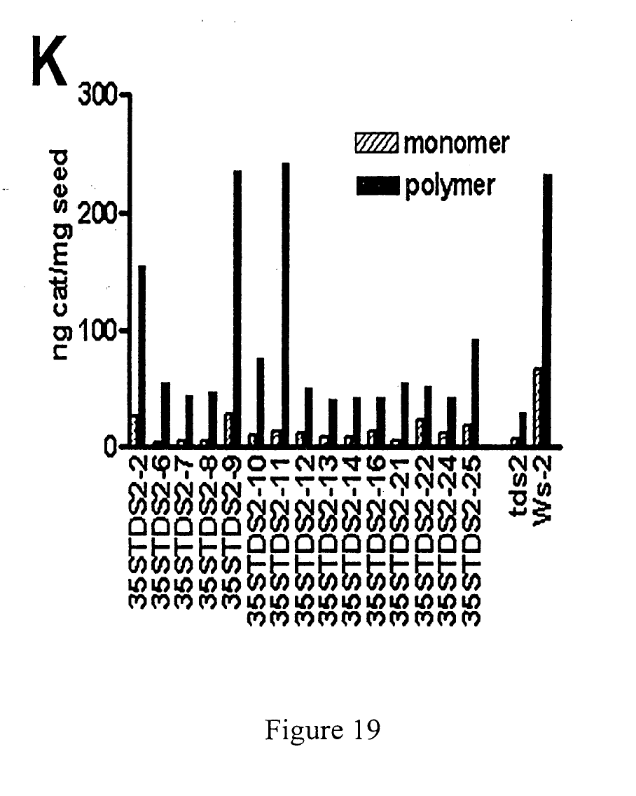

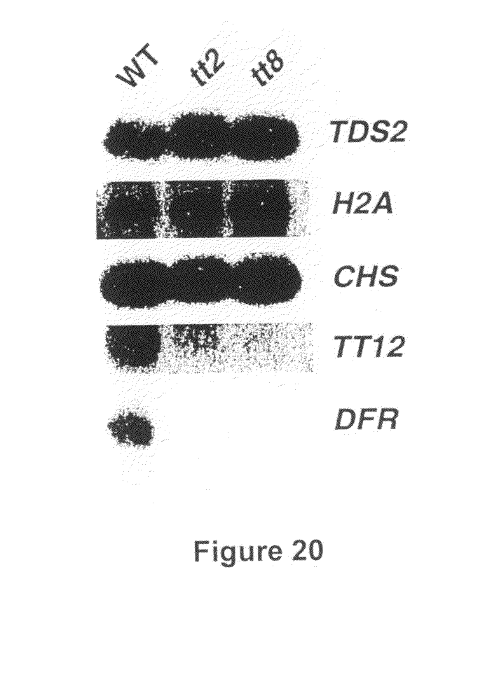

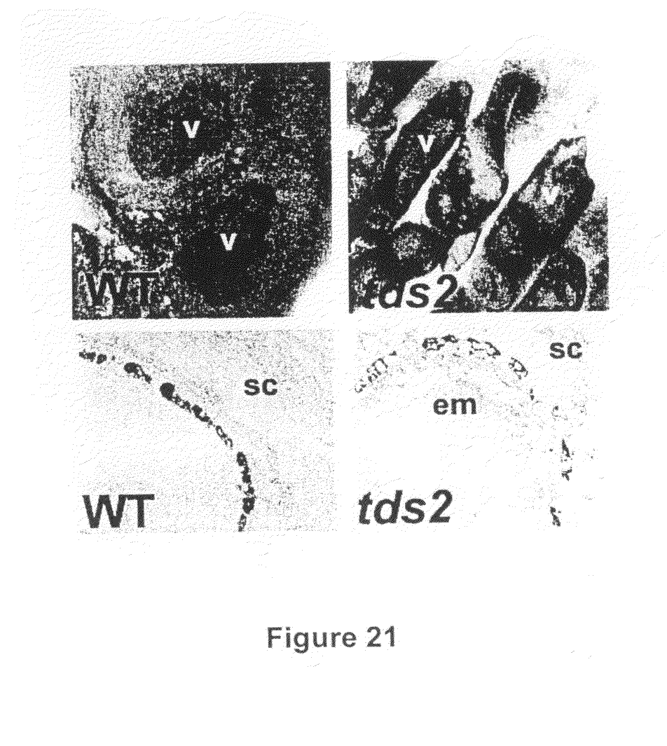

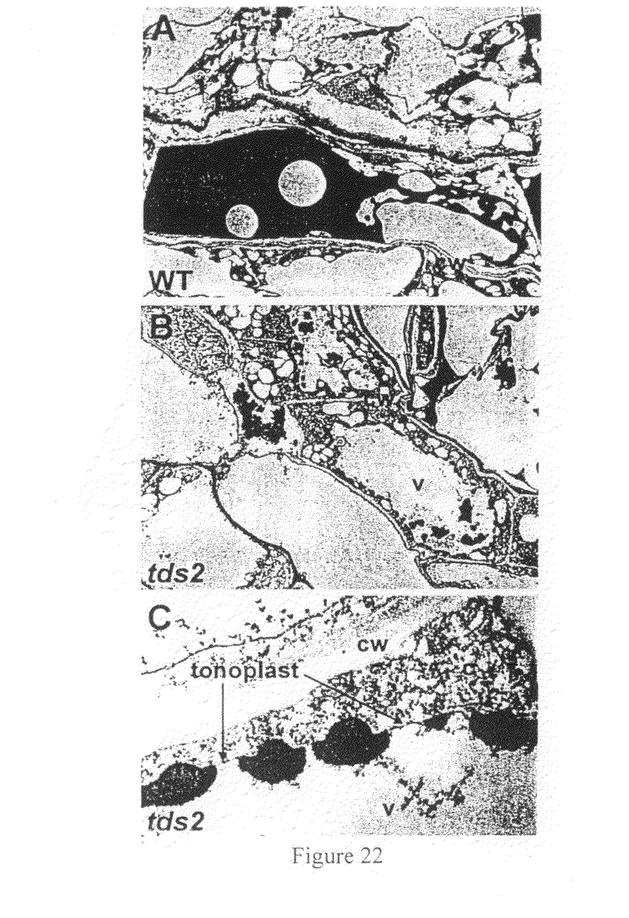

Much of the analysis of PA biosynthesis in Arabidopsis has been confined to analysis of the expression of genes such as LDOX and BAN, which are required for the formation of PA monomers, and their transcriptional control by TT2 and TT8 regulators. We were interested to correlate the gene expression patterns with PA metabolites themselves, namely the formation of monomer epicatechin, and to consider later steps in PA biosynthesis that involve the formation of PA polymer. Wild-type developing siliques from the torpedo stage of embryo development through to dehiscence were harvested, and analysed for the amounts of monomer and polymer present. Under the conditions used, the developing seeds were at the torpedo to upturned-U stage of development 6 to 8 days after flowering. Dehiscence was at about 16 to 18 days after flowering. Therefore, each of the ten samples shown in FIG. 14A represents approximately one day in the maturation of the seed. 70% acetone can be used to extract PA precursors (monomers) and PA (polymers). Extraction with ethyl acetate separates monomers (and some dimers) from polymers, and subsequent removal of the remaining acetone yields the polymers in aqueous solution. The monomer epicatechin was present throughout all stages of development (FIG. 14A), with a peak in its concentration at the upturned-U stage of development, after which a decline was observed. The concentration of polymer also increased until a peak at the upturned-U stage of development, after which a decline in extractable polymer was observed (FIG. 14A). A decrease in polymer might indicate an inability to extract polymer due to oxidation, cross-linking to insoluble cell components or further polymerisation.

Epicatechin and PA were separated using TLC and detected with 0.1% DMACA. FIG. 14B shows the time course of epicatechin and PA formation during seed maturation. The ethyl acetate fractions of PA extracts contained one major intermediate that reacted with DMACA, which has the same mobility as epicatechin (FIG. 14B). This intermediate was shown to be epicatechin by HPLC-mass spectrometry and also HPLC and TLC retention times (see above). However, when the aqueous fractions of PA extracts were separated by TLC, a number of DMACA reacting intermediates were observed, representing PA polymers of differing length, all of which would have been measured in the DMACA plate assay as polymer (FIG. 14A). The series of catech in monomer, dimer and trimer standards were shown to demonstrate the relative mobility of these intermediates, indicating that the solvent used could separate these compounds, and therefore other related series of isomers. Longer oligomers and the corresponding series of epicatechin dinners and trimers were not available for analysis. The standard PA was isolated from the leaves of the legume Onobrychis viciifolia and purified using an LH-20 column (Tanner et al., 1994), which tended to preferentially purify PA polymers depleted of short oligomers. Consequently, Onobrychis PA did not move from the origin using this solvent system. From the TLC of PA oligomers (FIG. 14B), it seemed that longer PA polymers that remain at the origin in younger samples were decreased in older samples, particularly dehiscent seed samples, indicating that longer polymers were becoming less extractable during seed development. This accounted for the apparent decrease in PA after the upturned-U stage of embryo development observed in FIG. 14A.

Although 70% acetone was not an optimal solvent for the extraction of anthocyanins, the aqueous fraction of the PA extractions also contained anthocyanins, which appeared as pink bands on the TLC. Interestingly, the amount of extractable anthocyanin increased steadily during seed development, reaching a maximum at dehiscence.

In the RT-PCR experiments described above, we had shown that the TDS6 gene was expressed at least until the upturned-U stage of development. We were therefore interested to examine later stages of development and, given the position of TDS6 in the PA biosynthesis pathway, to correlate TDS6 gene expression with the formation of epicatechin monomer or PA polymer. FIG. 14C shows that expression of TDS6 occurred throughout seed development, with the steady state level of TDS6 mRNA slowly declining until dehiscence, relative to the expression of the histone H2A gene.

### Example 9

### Wild-Type and tds6-1 Developing Seedcoat Endothelial Cells have Double Membrane Vesicles

TEM was used to study the pattern of PA deposition in wild-type and tds6-1 developing seeds. FIG. 15 shows sections of wild-type and tds6-1 developing endothelial cells stained with osmium tetroxide, which reacts with PA and related intermediates. At the torpedo stage of development, the wild-type cell was almost entirely occupied by the PA containing vacuole (FIGS. 15B and D). Within the vacuole, in addition to the uniformly grey areas of accumulated PA, there were spherical and irregular shaped regions that did not react with osmium. Sections of tds6-1 seeds at the same stage of development (FIGS. 15A and C) showed that the vacuole had developed to the same size as in wild-type cells, but the osmium reacting material was confined to the periphery of the vacuole and to regions surrounding smaller vesicles within the lumen of the vacuole. At higher magnifications of tds6-1 (FIGS. 15E and F), it was evident that the vesicles that fuse with the tonoplast did not contain osmium reacting material, so they probably did not contain PA related intermediates. The content of the vesicles was similar in appearance to that of the cytoplasm. It was also clear that the vesicles were surrounded by two membranes (FIG. 15E), the outer membrane apparently fusing with the tonoplast, leaving the single membrane bound vesicle within the lumen of the vacuole (FIG. 15F). These vesicles associated with each other and were surrounded by an accumulation of PA.

Due to the differences in the stage of PA formation evident in WT and tds6-1 seeds at the torpedo stage of development (FIG. 15), we were interested to observe earlier stages of PA development in wild-type seeds. At the two terminal cell stage of embryo development, the developing vacuole was smaller in size, more amorphous in shape, and its contents appeared more granular than at later stages of embryo development. There were numerous small vacuoles within the cell, that did not contain PA-related intermediates, some of which appear to be engulfed by the main vacuole. PA was present as a dark region around the periphery of the vacuole, and adjacent to internalised vesicles, similar in appearance to, but smaller than, the tds6-1 vacuole at the torpedo stage of development.

The evidence indicated that TDS6 was an enzyme involved in the formation of PA, downstream of BAN, which suggested it might be involved in polymerisation or the formation of PA extension units. Polymerisation is thought to occur in the vacuole, but TDS6 did not contain an N-terminal or C-terminal signal peptide that would direct the TDS6 protein to the secretory pathway that is usually a prerequisite for transport to the vacuole. However, vacuolar targeting signals may also be located in an exposed region of the mature protein, such as the signals used by phytohemagglutinin and legumin (Marty, 1999). This type of vacuolar signal sequence cannot be defined by homology and needs to be identified experimentally for each protein. At present there is no clearly definable general consensus sequence for plant vacuolar targeting (Vitale and Raikhel, 1999).

Although the secretory pathway from the ER to the vacuole has been well documented in plants, the CVT route has only been described in detail in mammalian cells (Reggiori and Klionsky, 2002) and in the yeast Saccharomyces cerevisiae (Abeliovich and Klionsky, 2001). The CVT pathway is an alternative route to the vacuole that does not require the protein to have a vacuolar targeting sequence and it is possible that TDS6 could be localised to the vacuole using this alternative route. The CVT pathway involves an enwrapping membrane sequestering a region of the cytosol, forming a double membrane vesicle, which is then targeted to the vacuole. The outer membrane of the vesicle then fuses with the tonoplast, allowing the release of a single membrane vesicle into the vacuolar lumen. The contents of the single membrane vesicle are eventually released into the vacuole lumen by vacuolar hydrolases. The yeast vacuolar hydrolase aminopeptidase I is an example of a protein that is transported to the yeast vacuole using this route (Klionsky, 1998). A characteristic of the CVT route is the observation of double membrane vesicles. The TEM sections of tds6-1 indicated that this process might be occurring in the PA accumulating endothelial cells in Arabidopsis. Vesicles with a double membrane were observed making contact with the tonoplast membrane in tds6-1 and wild-type sections, and numerous single membrane vesicles within the vacuolar lumen were also observed. The region surrounding the single membrane vesicles reacted strongly with osmium, suggesting it was a site for PA formation within the vacuole, perhaps being required to increase the surface area to volume ratio of the developing vacuole. The double membrane vesicles did not appear to contain PA or related intermediates. Rather, the contents of the vesicles were similar to that of the cytoplasm in appearance, suggesting that it may originate from the cytoplasm. Autophagic processes involving recycling of the cytoplasm and its contents overlap with some of the steps in the CTV pathway and may be required as the vacuole that accumulates PA occupies an increasing proportion of the cell volume as it develops. Recently, Smertenko et al. (2003) documented the progressive expansion of the vacuole in embryonic cells as an integral part of programmed cell death (PCD). The large central vacuole formed normally lyses in the death phase of PCD. It is possible that the Arabidopsis endothelial cells are utilising only part of the PCD process, to create the PA containing vacuole.

### Example 10

### Isolation of a White Clover TDS6 Orthologue

Nucleotide sequences of TDS6 or orthologues were obtained from analysis of ESTs from the legumes soybean, Lotus japonicus and Medicago truncatule. From an alignment of these sequences, degenerate oligonucleotides were designed to amplify the full amino acid coding region of other legume TDS6 orthologues. The sequences of the 5′ and 3′ oligonucleotides were

| | |
|---|---|
| LegTDS6-5′: | GGATCCATGGCIASTGAAAIIGTTTTGGTTGATG; |
| and | |
| LegTDS6-3′: | CGGATCCTTCACTTGGACAAYTCCTSYGAGA; |

respectively.

RNA was isolated from the tannin-containing flowers of white clover and reverse transcribed to cDNA using an oligo dT primer. The white clover TDS6 orthologue was then amplified with the LegTDS6-5′ and LegTDS6-3′ primers. The DNA product was cloned and sequenced. The sequence of the amplified product [SEQ ID NO: 5] was most closely related to the TDS6 orthologues from other legumes and contained all four conserved motifs found in TDS6 proteins.

### Example 11

### Cloning of the TDS2 Gene

The tds2 mutant was created by insertion of a T-DNA from the vector pGV3850:1003 encoding the Nptll gene (Velten and Schell, 1985; Feldmann and Marks, 1987) and was identified from pool No. 2540 of the Feldmann collection of mutants as described above. The mutant plant possessed multiple copies of the T-DNA insert at two different insertion sites. A tds2 mutant plant was crossed to a corresponding wild-type plant (ecotype Ws-2), and the PA biosynthesis phenotype of seed from 39 F2 plants was analysed with DMACA. The F2 plants segregated 26 wild-type: 13 tds2 mutant. All 13 of the F₂ plants with the tds2 mutant genotype were resistant to kanamycin, showing co-segregation of the tds2 mutation with the T-DNA conferring kanamycin resistance.

To Identify the plant DNA sequences flanking the T-DNA insertion, DNA was isolated from tds2 leaves, digested with NdeI or Bst11071, circularised by ligation with T4 DNA ligase, and used for inverse PCR as described by Ponce et al. (1998). Sequence analysis showed that one T-DNA in the tds2 mutant plant was inserted in the third exon of a gene designated At3g17980 on chromosome III. The gene At3g17980 and protein shall be referred to as 7DS2 and TDS2, respectively, hereafter.

### Example 12

### Structural Analysis of the TDS2 Gene and Molecular Complementation of the TDS2 Mutation

The gene annotation in The Institute for Genomic Research (TIGR) database suggested that the TDS2 gene consisted of three exons and two introns. This is the gene structure shown in FIG. 16A. The TDS2 gene encodes a predicted protein of 177 amino acids, which includes a C2 conserved domain, also known as a phospholipid-binding domain (Nalefski and Faike, 1996). The C2 domain was identified by cDART (conserved Domain Architecture Retrieval Tool), available from the NCBI (National Centre for Biotechnology Information) web site http://www.ncbi.nlm.nih.gov/. The relative position of the C2 domain in the TDS2 protein is shown in FIG. 16B. The sequence encoding the C2 domain extends across two Introns and into the third exon. The T-DNA is inserted in exon three, approximately 63 nucleotides (21 amino acids) from the end of the gene/protein. This is sufficient to cause the tds2 mutant phenotype, even though the C2 domain is intact. FIG. 16C shows the homology of the TDS2 C2 domain with the protein kinase C (PKC) C2 domain, spanning 84 amino acids towards the N-terminal region of the protein. Aspartate residues involved in the binding of Ca²⁺ by PKC oil (Edwards and Newton, 1997) are conserved in the TDS2 protein.

The NCBI database was searched using the program PSI BLAST to search for proteins having homology to the predicted TDS2 protein. The Arabidopsis genome encodes nine other proteins of approximately 160 to 180 amino acids in length that share from 84 to 50% amino acid identity with TDS2, encoded by genes Artg48590, At1g73580, At5g37740, At1g66360, At1g70790, At1g70810, At2g01540, At1g23140 and At1g70800, all of undefined function. A number of other C2 domain containing proteins share some homology with TDS2, only within the C2 domain, and include three ARF-GAP proteins of 370 amino acids At4g05330, At4g21160 and At3g07940 (100.86 and 52% identity within C2 domain). These are annotated as GTPase activating proteins. These proteins have an N-terminal 160 amino acid ARF-GAP domain and single C-terminal C2 domain.

There are few TDS2 ESTs available in the TIGR Arabidopsis database, so to check the annotation of the TDS2 gene, we amplified TDS2 cDNAs from Col-7 silique RNA. When the PCR products were cloned and sequenced, we found that of thirteen cDNAs analysed, eleven had the same sequence as the annotated sequence. The remaining two cDNAs showed evidence of incorrect splicing of the first intron, creating a truncated TDS2 protein.

C2 domains are common to a number of classes of proteins and are functional protein units that can appear in any part of a protein, in combination with other conserved domains, which together define the function of the protein (Johnson et al., 2000). FIG. 17 shows the relative positions of C2 domains in other proteins, obtained from cDART, indicating that TDS2 is unlike any other C2 domain-containing proteins, since it appears to have only a single C2 domain. A search for TDS2 homologs in the NCBI database shows that TDS2-like proteins of 160-180 amino acids in length are unique to plants, being identified for example in Arabidopsis, (Accession Nos. At1g48590. At1g73580, At5g37740, At1g66360, At1g70790. At1g70810, At2g01540. At1g23140, At1g70800. At5g47710), Oryza sativa (BAC79554.1) and the resurrection grass Sporobolus stapfianus (CAA71759.1).

Molecular Complementation of the tds2 Mutation

To confirm the role of TDS2 in PA biosynthesis, a 35S-TDS2 construct was made that might complement the mutant phenotype in tds2 plants. The 35S-TDS2 construct was made by PCR amplifying the coding region of the TDS2 gene from A. thaliana ecotype Col7 genomic DNA using primers TDS2 ATG EcoRI (5′-GCTCTAGAGTGAGTAAGGAAGAAATAATCAGGAAC-3′) and TDS2-3′ (5′-CCGAGCTCCCATGCTGTTACTTGGTTTAGTTC-3′) and inserting the coding region into the binary vector pSAN1 (Watson, unpublished). The vector pSAN1 carries the BAR gene that confers phosphinothricin (PPT) resistance and therefore could be used to transform the tds2 plants that were kanamycin resistant. After sequencing demonstrated the cloned sequence was correct, the 35S-TDS2 construct was transformed into the tds2 mutant background using Agrobacterium and a modified vacuum infiltration method (Bechtold et al., 1993). Resulting T₁ seed were germinated on MS media containing PPT (10 mg/ml) and kanamycin (50 mg/ml). Resistant T₁ plants were grown, T₂ seed collected and stained using 1.0% (w/v) DMACA (Abrahams et al., 2002). FIG. 18 shows the phenotype of wild-type Ws-2 compared with that of tds2 seed, without DMACA stain (FIG. 18A, C) and with DMACA stain (FIG. 18B, D). FIGS. 18F, H and J show pools of DMACA stained T₂ seed from three of the complemented tds2 progeny, namely ³⁵S-TDS2-2, 35S-TDS2-9 and ³⁵S-TDS2-10. Because the seed coat is maternal tissue, we expected to observe that the complemented phenotype conferred by the transgene in the T₂ seed, not the T₁ seed. The T₂ pools of seed showed differing levels of staining with DMACA, with some patches of staining evident, presumably due to differing levels of expression of the 35S-TDS2 gene. To demonstrate that the level of staining paralleled the production of PA, both PA precursor (monomer) and PA were extracted from pools of T₂ seed. Wild-type seed produced approximately 230 ng of PA polymer (catechin equivalents per mg seed), whereas tds2 produced 30 ng (catechin equivalents per mg seed) of PA polymer. The 35S-TDS2 T2 seed produced between 40 and 240 ng (catechin equivalents per mg seed) PA polymer, which could account for the differences in staining observed with DMACA (FIG. 19). These data demonstrated that loss of TDS2 activity in the mutant plants caused the PA-deficient phenotype.

Southern blot analysis of the ³⁵S:TDS2 complemented lines showed that the plants contained both BAR and Nptll genes associated with each of the two T-DNA's present in their genomic DNA, one from the mutating T-DNA (Nptll) and one from the complementing T-DNA (BAR). T₂ seed from lines 35S:TDS2-10, -14 and -25, which each appeared to have a single copy of the complementing T-DNA by Southern blot analysis, were germinated on MS media containing both PPT (10 mg/L) and kanamycin (50 mg/L), and were found to segregate for PPT resistance:sensitive in a 3:1 ratio. As expected, the control tds2 seed germinated only in the absence of PPT. T₂ seed from line 35S:TDS2-10, selected for further analysis because K appeared to have a single copy of the BAR gene by Southern analysis, was germinated on MS media without selection and individual plants grown to produce populations of T3 seed, each of which was stained with DMACA to observe segregation of complemented WT and tds2 phenotypes. Of 36 T2 plants, 31 were PPT resistant and 5 PPT sensitive. The 5 sensitive plants all produced uniform tds2 mutant seed. Moreover, the PPT sensitive T2 plants did not have BAR hybridising bands on Southern blot analysis. We concluded from the complementation analysis that we had cloned the TDS2 gene involved in the synthesis of PA.

### Example 13

### Expression of the TDS2 Gene

Since PA accumulates only in the endothelial cell layer of Arabidopsis seeds, the pattern of expression of the TDS2 gene was determined in tissues including leaf, stem, flowers and developing siliques. The expression of the 7TJS2 gene was analysed by reverse transcription-PCR (RT-PCR) using RNA harvested from Col-7 plants. To determine the stage of embryo development in developing siliques, the length of each developing silique was measured when harvested, and the extent of embryo development later confirmed by microscopic examination of stained sections. Primers for the RT-PCR analysis were designed to amplify across intron 2 of the TDS2 gene (primers TDS2-for 5′-1-3′ and TDS2-3′ above), giving products of 291 bp derived from RNA or 372 bp from genomic DNA templates, to allow for discrimination between amplification products derived from RNA or residual genomic DNA in the RNA samples. The number of rounds of amplification was limited to fifteen, based on optimised conditions. Primer annealing temperatures were determined using a gradient PCR block (Hybaid). As a control, histone H2A sequences were amplified using primers as in Devic et al. (1999). Products were separated on an agarose gel, the gel blotted onto N⁺ membrane (Hybond) using 0.4 M NaOH, and probed with DNA fragments of 7DS2 and H2A amplified from genomic DNA, which were sequenced to confirm their identity.

TDS2 mRNA was detected in flowers and young siliques (FIG. 20A). TDS2 expression was not detected in leaves, stems or older siliques. The expression of TDS2 was compared with that of TT12, which encodes a MATE transporter involved in PA synthesis (Debeaujon et al., 2001). TT12 expression was confined to developing siliques, between the two terminal cell and torpedo stages of development, with no expression detected in older samples, as shown in FIG. 20A. TDS2 expression was much more restricted than 7T72, being detectable only in siliques at the two terminal cell stage of embryo development (FIG. 20A).

To determine whether TDS2 expression was regulated by TT2 or TT8, RT-PCR was used to monitor the expression of TDS2 mRNA in tt8 and tt2 mutant backgrounds. FIG. 20B shows expression of CHS, DFR, TT12, TDS2 and histone H2A in wild-type, tt2 and tt8 developing siliques. The CHS gene was included because it was not regulated by TT2 or TT8, as indicated by similar levels of CHS product in wild-type, tt2 and tt8 samples. Both DFR and TT12 genes were not expressed in the tt2 and tt8 mutants, indicating that their expression was regulated by both TT2 and TT8 proteins, as had previously been observed (Nesi et al., 2001). In contrast, the TDS2 gene was equally expressed in wild-type, tt2 and tt8 siliques, indicating that TDS2 was not regulated by either TT2 or TT8, in a similar fashion to TDS6 as described above.

### Example 14

### Microscopic Examination of the Tds2 Seed Phenotype

Light microscopy was used to examine tds2 and wild-type developing seed stained with DMACA. FIGS. 21A and B show seeds dissected to remove the embryo, stained with DMACA. The endothelial cell layer was easily recognized by its staining with DMACA. In endothelial cells of wild-type seeds, the DMACA appeared to be contained within the vacuole, whereas in tds2 endothelial cells, DMACA stain appeared to be excluded from the vacuole, or around its periphery. Sections of osmium tetroxide treated developing seeds were also analysed for PA. In sections of wild-type seeds, the PA in endothelial cells reacted with osmium tetroxide to produce a dark precipitate that occupied a large proportion of the cell, with some very dark and contrasting light areas observed within the same cell (FIG. 20C). The osmium-reacting material in the endothelial cells of tds2 seed, however, appeared to be confined to the periphery of the vacuole. Thus, two different staining methods for PA and related intermediates suggest that PA intermediates were not being transported to the vacuole in the tds2 mutant.

Transmission electron microscopy was used to study the ultra-structural detail of PA deposition in wild-type and tds2 developing seeds. FIG. 22 shows sections of wild-type and tds2 developing endothelial cells stained with osmium tetroxide. At the torpedo stage of embryo development, the wild-type cell was almost entirely occupied by the PA containing vacuole (FIG. 22A). Within the vacuole, in addition to the uniformly grey areas of accumulated PA, there were spherical and irregular shaped regions that did not react with osmium tetroxide. Sections of tds2 seeds at the same stage of development showed a large central vacuole that had osmium reacting regions around the edge of the vacuole. Higher magnifications of the same section (FIG. 22C) showed distinctive structures located at the tonoplast, which appeared to have fused with the membrane, but not released the contents into the vacuole. A small amount of osmium-reacting material was also located within the lumen of the vacuole in the tds2 mutant.

### Discussion.

### The TDS2 Protein is Necessary for the Accumulation of PA in Arabidopsis Seed Coat Endothelial Cells.

The results presented here demonstrate that the TDS2 gene encodes a protein necessary for the accumulation of PA within the vacuole of Arabidopsis endothelial cells. The tds2 mutant phenotype was complemented using a 35S:TDS2 construct, as shown by DMACA staining of mature T₂ seed from transformed plants, and extraction and quantitation of PA and monomer from mature T₂ seed. The co-segregation of the complementing T-DNA with the restored wild type phenotype in T seed was also demonstrated. The TDS2 protein encodes a C2 domain, which has all of the conserved amino acids required for Ca²⁺ binding, which is a prerequisite for the binding of phospholipids or membrane associations (Stahelin and Cho, 2001). The expression of the TDS2 gene in developing siliques correlates with the synthesis of PA in the seed coat but not other tissues in Arabidopsis. Using TEM, we have shown that PA related intermediates accumulate around the periphery of the vacuole in discrete vesicles. The vesicles appear to have fused with the tonoplast, but not released the contents of the provacuole into the lumen of the vacuole, suggesting that TDS2 is involved in this process.

### The TDS2 Protein Encodes a C2 Domain Found in Membrane-Associated Proteins

The C2 domain, or phospholipid binding domain, is a membrane targeting domain found in a number of types of protein involved in signal transduction and membrane trafficking, being present in synaptotagmin, protein kinases, GTPases and phospholipases (Nalefski and Falke, 1996). Many C2 domains bind phospholipid signal molecules in a Ca²⁺ dependent manner, and thereby play an important role in Ca²⁺ dependent targeting of proteins to membranes, such as classical PKCs (Verdaguer et al., 1999). More recently, the binding of phospholipids to a novel class of protein kinase C has been shown to lack the calcium dependence of classical PKCs (Ochoa et al., 2001). These novel PKCs have structural changes in the Ca²⁺ binding pocket and maintain only two of the aspartate residues present in other C2 domains (Ochoa et al., 2001).

The Role of Ca²⁺ and Phospholipids in the Association of C2 Domains with Membranes.

Perception of Ca²⁺ signals requires interaction of Ca²⁺ with proteins in a concentration-dependent manner. Ca²⁺ regulatory motifs include EF hands, annexin folds and C2 domains (Kopka et al., 1998). The C2 domain can be divided into three subdomains, A, B and C. The A subdomain consists of a D-P—Y—V-K motif located on the N-terminal side of the core region, the B subdomain contains a K-X-K(R)-T motif, and the C subdomain, represented by the segment L-N—P—X—W—N—(X)-E-X—F—X—F, which is C-terminal to the basic core (Kopka et al., 1998). The crystal structure of synaptotagmin IA (Sutton et al., 1995) defined four amino acids D172/D178 and D230/D232 which create a Ca²⁺ binding sphere. NMR spectroscopy revealed that D238 also contributes to Ca²⁺ binding (Shao et al., 1996). The predicted TDS2 protein has all of the amino acid residues present in the C2 domain necessary for Ca²⁺ binding. In yeast, fusion of the prevacuole with the lysosome involves the association of v-SNAREs, t-SNAREs, GTPase and SNAP proteins in a complex, to bring about the fusion of the vesicle with the lysosome (Lodish et al. 2000). Since TDS2 has all of the conserved amino acids required for Ca²⁺ binding, and appears to be involved in the fusion or release of PA-related intermediates into the vacuole, TDS2 probably plays a role in the fusion events associated with the accumulation of PA-related intermediates in the vacuole. Proteins with homology with TDS2 might therefore play a role in similar processes involved in the accumulation and trafficking of proteins or other secondary metabolites, including the vacuole or secretion from the cell at the plasma membrane. Blatt and Thlel (2003) suggest that at least three different pools of vesicles are available for delivery to the plasma membrane alone.

Structural and binding analyses of C2 domains have determined the Ca²⁺ binding stoichiometry, geometry and affinity for various C2 domains (Stahelin and Cho, 2001). However the precise role of C2 bound Ca²⁺ ions in membrane targeting is unknown. Ca²⁺ may associate directly with C2 domains in proteins to modulate activity, and binding could induce a conformational change (Zheng et al., 2000) to initiate membrane binding, or a proteins' activation for catalysis (e.g. GTPases). Alternatively, Ca²⁺ binding may change the surface charge, potential or shape of a membrane. The binding of phospholipids to C2 domains is often dependent on the binding of Ca²⁺ to the C2 domain (Davletov and Sudhof, 1993; Edwards and Newton, 1997; Verdaguer et al., 1999; Johnson et al., 2000). Biochemical studies have localised the phospholipid-binding site to the area surrounding the Ca²⁺ binding site (Edwards and Newton, 1997; Medkova and Cho, 1998; Nalefski and Falke, 1998).

### Vesicles are Involved in the Transport of Secondary Metabolites to the Vacuole.

Early cytological studies of the formation of PA-related provacuoles derived from the ER suggested that the intermediate being transported by the provacuoles was PA polymer (Baur and Wilkinshaw, 1974; Chafe and Durzan, 1973; Parham and Kaustinen, 1977). However, biochemical analyses of the intermediates in tds2 indicate that tds2 makes small amounts of the monomer epicatechin (Abrahams et al., 2002) and polymer, suggesting that the provacuoles may transport epicatechin, and not polymers. If tds2 mutants presumably have all of the enzymes necessary for the formation of PA, and yet do not make wild type amounts of PA, suggesting that spatial separation of intermediates is sufficient to prevent the synthesis of PA. An advantage of the spatial separation of intermediates is that it offers another level of control over the polymerisation of PA, apart from regulation of gene expression (Nesi et al., 2000; Nesi et al., 2001), by maintaining intermediates in discrete compartments until fusion with the vacuole. A characteristic feature of the secretory system is the recycling of vesicles back to the Golgi and ER after the transport of the vesicle contents. The vesicles present in tds2, which appear to be caught at the vacuolar membrane, are unavailable for reuse. It is possible that the inappropriate accumulation of PA related intermediates in the cytoplasm due to a lack of vesicles causes feedback inhibition of enzymes such as DFR, LDOX or BAN. This might explain the observation by Nesi et al (2001) that ectopic expression of TT2, and therefore induction of DFR, LDOX and BAN gene expression, and presumably protein synthesis, did not lead to the formation of epicatechin as expected. RT-PCR analysis indicates that expression of TDS2 is not dependent on TT2 or TT8, so there are at least two genes, TDS2 and TDS6 (Abrahams et al., 2003b) involved in the synthesis of PA in Arabidopsis that are regulated by proteins other than TT2 and TT8.

This work demonstrates that in addition to the established role of vesicles in the transport of proteins to the vacuole (Lodish et al., 2000), vesicles are also Involved in the transport of PA intermediates, and possibly other secondary metabolites, to the vacuole. A likely sequence of events involves the synthesis of epicatechin in the cytoplasm side of the ER membrane by enzymes such as DFR, LDOX and BAN, transport of epicatechin to a localised region of the ER lumen, possibly by TT12 (Debeaujon et al., 2001), and budding of a vesicle from the ER which contains epicatechin. The vesicle is relocated to the main vacuole, where vesicle fusion and release of its contents into the lumen of the vacuole occurs. Extension units might be transported to the vacuole via a related series of events. Presumably, polymerisation then takes place in the vacuole by a polymerising enzyme. Alternatively, polymerisation may take place within the vesicle, with subsequent deposition of PA into the vacuole. This sequence of events is consistent with a number of lines of evidence. Double mutant analysis indicates that TDS2 is required after LDOX, BAN and TDS3 in the PA pathway (Abrahams et al., 2003). The ban tt12 double mutant is reported to resemble ban seeds but with a paler appearance (Debeaujon et al., 2001), so it is not clear if TT12 transports the epicatechin product of BAN or not it is not yet known where TT12 is placed in relation to TDS3 or TDS2 in the pathway. Early immuno-cytological and biochemical experiments provide evidence that the phenylpropanoid enzyme chalcone synthase, an enzyme common to the synthesis of PA and related monomeric flavonoids, localises to the cytoplasmic side of the ER (Hrazdina et al., 1987) and that the ER is the site of phenylpropanoid and flavonoid metabolism in petals of Hippeastrum (Wagner and Hrazdina, 1984; Hrazdina and Wagner, 1985). More recent studies indicate that the Arabidopsis enzymes chalcone synthase, chalcone isomerase and DFR are Involved in specific protein-protein interactions thought to be necessary for substrate channeling (Burbulis and Winkel-Shirley, 1999).

Membrane fusion events are characterised by being specific, rapid and reversible, so that proteins can be reused in another cycle of vesicle trafficking. Since the provacuole fusion events occur in fractions of seconds (Lodish et al., 2000), the usual methods used for the preparation of sections often fail to detect vesicles in the process of fusing, particularly methods that involve osmium tetroxide treatment and dehydration of the seed before sectioning. The appearance of prevacuoles in the tds2 TEM sections and the observation that tds2 is unable to make PA, provides evidence that prevacuoles do indeed fuse with the tonoplast, and of the role of TDS2 in trafficking of PA intermediates to the vacuole. There is cytological evidence that PA intermediates accumulate in the ER lumen, regions of which bud off to form provacuolar compartments which then fuse with the vacuole (Chafe and Durzan, 1973; Baur and Walkinshaw, 1974; Amelunxen and Heinze, 1984; Hilling and Amelunxen, 1985). Other secondary metabolites such as the alkaloid berberine have also been observed in various stages of the same process (Amann et al., 1986; Bock et al., 2002). The flavonoid 3-deoxyanthocyanidin, a phytoalexin synthesized by Sorghum bicolour in response to infection by a fungal pathogen, also appears to be transported to the cell membrane via vesicles (Snyder and Nicholson, 1990).

### TDS2-Related Proteins.

Most proteins containing C2 domains also contain other protein regulatory modules, for example those involved in enzyme catalysis in PKCs, phospholipases, and GTPases. TDS2 is unusual among C2 domain containing proteins in that it has only a C2 domain, suggesting that TDS2 interacts with another protein during the vesicle fusion process. The Arabidopsis protein BAP1 (BON1 ASSOCIATED PROTEIN) is similar in structure to TDS2, in that it has a single C2 domain and is 192 amino acids in length (Hua et al., 2001), but it is not one of the Arabidopsis homologs of TDS2 previously discussed. BAP1 was identified in a yeast two-hybrid screen for proteins that interact with BON1 (BONZAI 1), a member of the copine family of proteins that function in the pathway of membrane trafficking in response to external stimuli (Hua et al., 2001). The association of BON1 with BAP1, and the tendency of C2 domain containing proteins to regulate trafficking processes, suggest that TDS2 is likely to function in association with another protein. Studies of the membrane-binding affinity of the C2 domain from PCK (Johnson et al., 2000) show that the isolated C2 domain itself has a higher affinity for membranes than the C2 domain within the context of the full-length protein. The existence of the TDS2 protein with only a C2 domain further demonstrates the independence of functional domains within proteins.

In many cases, for a single yeast vesicle transport gene, multiple genes exist in Arabidopsis. Arabidopsis orthologs of yeast proteins have been characterised by complementation of yeast mutants (Zheng et al., 1999; Sato et al, 1997). Unfortunately, Arabidopsis mutants such as vacuoleless 1 (Rojo et al., 2001) may only be maintained in the heterozygous state, indicating that they are essential to plant cell function (Bassham and Raikhel, 2000; Bassham et al., 2000). In contrast, the tds2 mutant phenotype is observed in the homozygous state, and does not appear to interrupt seed development or viability, presumably because the formation of a PA specific vacuole and PA are not essential to plant cell function. The endothelial cell layer itself is dispensable, since mutants of TT16 have partially ablated endothelial cells, which does not appear to adversely affect seed development or viability (Nesi et al., 2002). The TDS2 gene appears to be expressed only in PA producing tissues and the tds2 phenotype confined to the PA vacuole of endothelial cells. Although there are nine other TDS2-like genes/proteins in Arabidopsis, they are either not expressed in the endothelial cells or may be unable to substitute for TDS2 function.

Study of secondary plant metabolites such as PA, being non-essential for the survival of the plant, can provide insights into the later stages of vesicle trafficking and fusion, and reveal the function of a class of proteins unique to plants. The involvement of vesicles formed by the endomembrane system used for the transport of metabolites, in addition to proteins, demonstrates the versatility of this type of transport pathway. It offers another level of control and degree of specificity to the formation of PA in Arabidopsis endothelial cells, beyond the transcriptional control of genes encoding enzymes involved in PA synthesis.

TABLE 1
Characteristics of the tds mutants

| | | | | | | Flavonols | |
|---|---|---|---|---|---|---|---|
| Back- | Muta- | Seed | | | | (seed) | |
| ground | tion | colour | Leafant | Seed PA | Flavanol | K | Q |
| Ws-2 | tds1 | tan | + | −^{b} | + | + | + |
| Ws-2 | tds2 | tan | +^{a} | −^{b} | + | + | + |
| Ws-2 | tds3-1 | tan | + | − | + | + | + |
| Ws-4 | tds3-2 | | | | | | |
| Ws-4 | tds4 | yellow | −/+ | spotty | +^{a} | + | + |
| Col-7 | tds5 | tan | + | −^{b} | + | + | + |
| Col-7 | tds6 | tan | + | +/−^{b} | + | + | + |
| Col-7 | tt8-4 | yellow | + | − | − | + | + |
| | tt8-5 | | | | | | |
| Col-7 | tt7-3 | tan | +^{a} | spotty | +^{a} | + | − |
| En | ban | brown | + | − | − | + | + |
| Ler | tt3 | yellow | − | − | − | + | + |
| (dfr⁻) | | | | | | | |
| Ler | tt4 | yellow | − | − | − | − | − |
| (chs⁻) | | | | | | | |
| Wild | | tan/dark | + | + | + | + | + |
| type | | tan | | | | | |

^{a}present but not WT.
^{b}positive at the basal end of the seed.
K kaempferol;
Q quercetin.
Col-7 Columbia;
Ws Wassilewskija;
Ler Landsberg erecta;
En Enkheim.

TABLE 2
Similarities of Arabidopsis TDS6 amino acid sequence with
orthologue TDS6 sequences and chalcone isomerase
sequences.

| | % amino acid identity to |
|---|---|
| | Arabidopsis TDS6 |
| Plant species | (Entire protein) |
| Clover | 64 |
| Cotton | 70 |
| Grape | 67 |
| Medicago | 62 |
| Poplar | 68 |
| Potato | 62 |
| Soybean | 61 |
| Tomato | 62 |
| Sorghum | 54 |
| Maize | 54 |
| Rice | 60 |
| Barley | 56 |
| Wheat | 58 |
| Lobiolly Pine | 59 |
| Verified Chalcone Isomerases | |
| Arabidopsis defined by mutation | 23 |
| Pueraria defined by CHI activity | 24 |
| Lucerne defined by 3D structure | 24 |
| Petunia A defined by mutation | 23 |
| Petunia B | 25 |
| At5g66220 | 23 |
| Maize defined by mutation | 24 |
| Chloroplast CHI-homologue | |
| Atlg53520 | 16 |

### REFERENCES

Abeliovich, H. and Klionsky, D. L. (2001). Autophagy in yeast: mechanistic insights and physiological function. Microbiol. Mol. Biol. Rev. 65, 463-479.
Abrahams, S., Lee, E., Walker, A. R., Tanner, G. J., Larkin, P. J. and Ash ton, A. R. (2003) The Arabidopsis TDS4 gene encodes leucoanthocyanidin dioxygenase (LDOX) and is essential for proanthocyanidin synthesis and vacuole development. Plant J. 35, 624-636.
Abrahams, 8., Tanner, G. J., Larkin, P. J. and Ashton, A. R. (2002) Identification and biochemical characterization of mutants in the proanthocyanidin pathway in Arabidopsis. Plant Physiol. 130, 561-576.
Albert, S., Delseny, M. and Devic, M. (1997) BANYULS, a novel negative regulator of flavonoid biosynthesis in the Arabidopsis seed coat Plant J. 11, 289-299.
Aubert, S., Gout, E., Bligny, R., Marty-Mazars, D., Barrleu, F., Alabouvette, J., Marty, F. and Douce, R. (1996) Ultrastructural and biochemical characterisation of autophagy in higher plant cells subjected to carbon deprivation: control by the supply of mitochondria with respiratory substrates. J. Cell Biol. 133, 1251-1263.
Ausubel, F. M., et al. 1987) Current Protocols in Molecular Biology, Wiley Interscience (ISBN 047140338).
Baba, M., Takeshige, K., Baba, N. and Ohsumi, Y. (1994) Ultrastructural analysis of the autophagic process in yeast detection of autophagosomes and their characterisation. J. Cell Biol. 124, 903-913.
Barry, T. N. and Reid C. S. W. (1985) Nutritional effects attributable to condensed tannins, cyanogenic glycosides and oestrogenic compounds in New Zealand forages. In Forage Legumes for Energy-Efficient Animal Production. Eds Barnes R F, Ball P R, Brougham R W, Martin G C, Minson D J.—251-259. USDA, ARS. Washington D.C.
Bassham, D. C. (2002). Golgi-independent trafficking of macromolecules to the plant vacuole. In Advances in Botanical Research 38, J. A. Callow, ed. (London: Academic Press), pp. 65-92.
Bassham, D. C. and Raikhel, N. V. (2000) Unique features of the plant vacuolar sorting machinery. Curr. Opin. Cell Biol. 12, 491-5.
Bassham, D. C, Sanderfoot, A. A., Kovaleva, V., Zheng, H. and Raikhel, N. V. (2000) AtVPS45 complex formation at the trans-Gokji network. Mol. Biol. Cell 11, 2251-65.
Baur, P. S, and Walfrinshaw, C. H. (1974) Fine structure of tannin accumulations in callus cultures of Pinus elliotti (slash pine). Can. J. Bot 52, 615-619.
Bechtold, N., Ellis, J., Pelletier, G. (1993) In planta Agrobacterium mediated gene transfer by infiltration of adult Arabidopsis thalian plants. CR Acad. Sci. 316, 1194-1199.
Beers, E. P. and McDowell, J. M. (2001) Regulation and execution of programmed cell death in response to pathogens, stress and developmental cues. Curr. Opin. Plant Biol. 4, 561-567.
Bethke, P. C. and Jones, R. L. (2000) Vacuoles and prevacuolar compartments. Curr. Opin. Plant Biol. 3, 469-475.
Blatt, M. R. and Thiel, G. (2003) SNARE components and mechanisms of exocytosis in plants. in, The Golgi apparatus and the plant secretory pathway. D G Robinson Ed. Sheffield, Sheffield Academic press. pp 208-237.
Bourque, J. E. (1995). Antisense strategies for genetic manipulations in plants. Plant Science 105, 125-149.
Burbulis, I. E. and Winkel-Shirley, B. (1999) Interactions among enzymes of the Arabidopsis flavonoid biosynthetic pathway. PNAS 96, 12929-12934.
Chafe, S. C. and Durzan, D. J. (1973) Tannin inclusions in cell suspension cultures of white spruce. Planta 113, 251-262.
Cole et al. (1965) in Monoclonal antibodies in cancer therapy, Alan R. Bliss Inc., pp 77-96.
Davletov, B., Perisic, O. and Williams, R. L. Calcium-dependent membrane penetration is a hallmark of the C2 domain of cytosolic phospholipase A2 whereas the C2A domain of synaptotagmin binds membranes electrostatically. J. Biol. Chem. 273, 19093-6.
Davletov, B. A. and Sudhof, T. C. (1993) A single C2 domain from synaptotagmin I is sufficient for high affinity Ca²⁺/phospholipid binding. J Biol. Chem. 268, 26386-90.
Debeaujon, I., Peeters, A. J. M., Leon-Kloosterziel, K. M. and Koornneef, M. (2001). The TRANSPARENT TESTA 12 gene of Arabidopsis encodes a multidrug secondary transporter-like protein required for flavonoid sequestration in vacuoles of the seed coat endothelium. Plant Cell 13, 853-871.
Delcour, J. A., Ferrelra, D., Roux, D. G. (1983) Synthesis of condensed tannins. Part 9. The condensation sequence of leucocyanidin with (+)catechin and the resultant procyanidin. J Chem Soc Perkin Trans 1, 1711-1717.

Devic, M., Guilleminot, J., Debeaujon, I., Bechtold, N., Bensaude, E., Koornneef, M., Pelletier, G. and Delseny, M. (1999) The BANYULS gene encodes a DFR-like protein and is a marker of early seed coat development. Plant J. 19, 387-398.
Dixon, R. A. and Steele, C. L. (1999). Flavonoids and isoflavonoids—a gold mine for metabolic engineering. Trends in Plant Sci. 4, 394-400.
Downey, M., Harvey, J. and Robinson, S. P. (2003) Analysis of tannins in seeds and skins of Shiraz grapes throughout berry development. Aust. J. Grape Wine Res. 9, 15-27.
Feinbaum, R. L. and Ausubel, F. M. (1988). Transcriptional regulation of the Arabidopsis thaliana chalcone synthase gene. Mol. Cell. Biol. 8, 1985-1992.
Feldmann, K. A. and Marks, D. (1987) Agrobacterium-mediated transformation of germinating seeds of Arabidopsis thaliana: a non-tissue culture method. Mol. Gen. Genet. 208, 1-9.
Filonova. L. H., Bozhkov, P. V., Brukhin, V. B., Daniel, G. Zhivotovsky, B. and von Arnold, S. (2000) Two waves of programmed cell death occur during formation and development of somatic embryos in the gymnosperm, Norway spruce.
Gleave, A. P. (1992) A versatile binary vector system with a T-DNA organisational structure conducive to efficient Integration of cloned DNA into the plant genome. Plant Mol. Biol. 20, 1203-7.
Gruber, M. Y., Ray, H., Auser, P., Skadhauge, B., Falk, J., Thomson, K. K., Stougaard, J., Muir, A., Lees, G., Coulman, B., McKersie, B., Bowley, S, and von Wettstein, D. (1999). Genetic systems for condensed tannin biotechnology. In Plant Polyphenols 2: chemistry, biology, pharmacology and ecology, G. G. Gross, R. W. Hemingway and T. Yoshida, eds. (London: Kluwer Academic), pp. 315-341.
Hadlington, J. L. and Denecke, J. (2000) Sorting of soluble proteins in the secretory pathway of plants. Curr. Opin. Plant Biol. 3, 461-468.
Hagerman, A. E. and Butler, L. G. (1981) The specificity of proanthocyanidin-protein interactions. J. Biol. Chem. 256, 4494-7.
Haseloff, J. and Gerlach, W. L. (1988) Nature 334, 586-594.
Hilling, B. and Amelunxen, F. (1985) On the development of the vacuole. II Further evidence for endoplasmic reticulum origin. Euro. J. Cell Biol. 38, 195-200.
Hrazdina, G. and Wagner, G. J. (1985) Metabolic pathways as enzyme complexes: evidence for the synthesis of phenylpropanoids and flavonoids on membrane associated enzyme complexes. Arch. Biochem. Biophys. 237.88-100.
Hrazdina, G., Zobel, A. M. and Hoch, H. C. (1987) Biochemical, immunological, and immunocytochemicai evidence for the association of chalcone synthase with endoplasmic reticulum membranes. Proc. Natl. Acad. Sci. USA. 84, 8966-70.
Huse et al. (1989) Science 246, 1725-1281.
Jauh, G. Y., Phillips, T. E. and Rogers, J. C. (1999) Tonoplast intrinsic protein isoforms as markers for vacuolar functions. Plant Cell 11, 1867-1882.
Jez, J. M., Bowman, M. E. and Noel, J. P. (2002). Role of hydrogen bonds in the reaction mechanism of chalcone isomerase. Biochemistry 41, 5168-76.
Jez, J. M., Bowman, M. E., Dixon, R. A. and Noel, J. P. (2000). Structure and mechanism of the evolutionarily unique plant enzyme chalcone isomerase. Nature Structural Biol. 7, 786-791.
Klionsky, D. L. (1998). Nonclassical protein sorting to the yeast vacuole. J. Biol. Chem. 273, 10807-10810.
Kohler and Milstein (1975) Nature 2546, 495-499.
Kohout S. C., Corbalan-Garcla, S., Gomez-Fernandez, J. C. and Falke, J. J. (2003) C2 domain of protein kinase C alpha: elucidation of the membrane docking surface by site-directed fluorescence and spin labeling. Biochemistry. 42, 1254-65.
Koornneef, M. (1990). Mutations affecting the testa colour in Arabidopsis. Arabld. Inf. Serv. 27, 1-4.
Kopka, J., Pical, C, Hetherington, A. M. and Muller-Rober, B. (1998) Ca²⁺/phospholipid-binding (C2) domain in multiple plant proteins: novel components of the calcium-sensing apparatus. Plant Mol. Biol. 36, 627-37.
Koupai-Abyazani, M. R., McCallum, J., Mulr, A. D., Lees, G. L., Bohm, B. A., Towers, G. H. N., Gruber, M. Y. (1993a) Purification and characterisation of a proanthocyanidin polymer from seed alfalfa (Medicago sativa Cv. Beaver). J. Agric. Food Chem. 41, 565-569.
Koupai-Abyazani, M. R., McCallum, J., Muir, A. D., Lees, G. L., Bohm, B. A., Towers, G. H. N, and Gruber, M. Y. (1993b). Developmental changes in the composition of proanthocyanidins from leaves of Sanfoin (Onobrychus viciifolia Scop.) as determined by HPLC analysis. J. Agric. Food Chem. 41, 1066-1070.
Kozbor et al (1983) Immunol. Today 4, 72.
Kristiansen, K. (1984). Biosynthesis of procyanidins in barley: genetic control of the conversion of dihydroquercetin to catechin and procyanidins. Carlsberg Res. Comm. 49, 503-524.
Kubasek, W. L., Shirley, B. W., McKillop. A., Goodman, H. M., Briggs, W. and Ausubel F. M. (1992). Regulation of flavonoid biosynthetic genes in germinating Arabidopsis seedlings. Plant Cell 4, 1229-1236.
Kuriyama, H. (1999) Loss of Tonoplast Integrity Programmed in Tracheary Element Differentiation. Plant Physiol. 121, 763-774.
Kuriyama, H. and Fukuda, H. (2002) Developmental programmed cell death in plants Curr Opin Plant Biol. 5, 568-573.
Larkin P. J. et al., (1996). Transgenic White Clover. Studies with the auxin responsive promoter, GH3, in root gravitropism and lateral root development. Transgenic Res. 5, 325-335.
Lazo, G. R. et al. (1991) A transformation-competent Arabidopsis genomic library in Agrobacterium. Bio/Technology 9, 963-967.
Lee J., et al. (1995). Sulphur amino acid metabolism and protein synthesis in yound sheep fed ryegrass pasture and two lotus cultivars containing condensed tannins. Aust J. Agric. Res. 46, 1598-1600.
Levanony, H., Rubin, R., Altschuler, Y., and Galili, G. (1992) Evidence for a novel route of wheat storage proteins to vacuoles. J. Cell Biol. 119, 1117-1128
Li, C, Davletov, B. A. and Sudhof, T. C. (1995) Distinct Ca²⁺ and Sr²⁺ binding properties of synaptotagmins. Definition of candidate Ca²⁺ sensors for the fast and slow components of neurotransmitter release. J. Biol. Chem. 270, 24898-902.
Li Y-G, et al. (1996) The DMACA-HCl protocol and the threshold proanthocyanidin content for bloat safety in forage legumes. J. Sci. Food Agric. 70, 89-101.
Lin, Y., Seals, D. F., Randall, S. K. and Yang, Z. (2001) Dynamic localization of Rop GTPases to the tonoplast during vacuole development. Plant Physiol. 125, 241-51.
Lister, C. E. and Lancaster, J. E. (1996). Developmental changes in enzymes of flavanoid biosynthesis in the skins of red and green apple cultivars. J. Sci. Food Agric. 71, 313-320.
Lodish, H., Berk, A., Zipursky, S. L., Matsudaira, P., Baltimore, D., and Darnell, J. E. (2000) Protein sorting: organelle biogenesis and protein secretion. In S. Tenney, ed. Molecular Cell Biology, 4^{th} Edition. WH Freeman and Company, New York, available online at www.ncbi.nlm.nih.gov/books.
Lyttleton (1971) NZ J. Agric. Res. 14, 101-107.
McNabb W. C., et al. (1993). The effect of condensed tannins in Lotus pedunculatus on the digestion and metabolism of methionine, cysteine, and inorganic sulphur in sheep. Brit. J. Nutrition 70, 647-661.
McPherson, M. J., et al. (1991) PCR A Practical Approach. IRL Press, Oxford University press, Oxford, United Kingdom.
Mansfield, S. G. (1994) Embryogenesis: Introduction. In J Bowman, ed. Arabidopsis: an atlas of morphology and development, Springer, New York, pp 351-361.
Marty, F. (1997) The biogenesis of vacuoles: insights from microscopy. In R A Leigh and D Sanders eds. Advances in Botanical Research. Academic Press, San Diego, Calif. 25, 1-42.
Marty, F. (1999) Plant vacuoles. Plant Cell 11, 587-599.
Matile, P. (1997). The vacuole and cell senescence. In Advances in Botanical Research 25, R. A. Leigh and D. Sanders, eds (San Diego: Academic Press), pp. 87-112.
Nalefski, E. A. and Falke, J. J. (1996) The C2 domain calcium-binding motif: structural and functional diversity. Protein Sci. 5, 2375-90.
Nesi, N., Debeaujon, I., Jond, C, Pelletier, G., Caboche, M. and Lepiniec, L (2000). The TT8 gene encodes a basic helix-loop-helix domain protein required for expression of DFR and BAN genes in Arabidopsis siliques. Plant Cell 12, 1863-1878.
Nesi, N., Jond, C, Debeaujon, I., Caboche, M. and Lepiniec, L. (2001). The Arabidopsis 772 gene encodes an R2R3 domain protein that acts as a key determinant for proanthocyanidin accumulation in developing seed. Plant Cell 13, 2099-2114.
Nielson, A. J. and Griffith, W. P. (1978) Tissue fixation and staining with osmium tetroxide: the role of phenolic compounds. J. Histochem. Cytochem. 26, 138.
Niezen J. H., et al. (1995). Growth and gastrointestinal nematode parasitism in lambs grazing either lucerne (Medicago sativa) or Sulla (Hedyserum coronarium) which contains condensed tannins. J. Agric. Sci. 125, 281-289.
Nonaka, G., Muta, M., Nishioka, I. (1983) Myricatin, a galloylflavanonol sulfate and prodelphinidin gallates from Myrica rubra. Phytochemistry 22, 237-241.
Ochoa, W. F., Garcia-Garcia. J., Fita, I., Corbalan-Garcia, S., Verdaguer, N. and Gomez-Fernandez, J. C. (2001) Structure of the C2 domain from novel protein kinase CO. A membrane binding model for Ca²⁺-independent C2 domains. J. Mol. Biol. 311, 837-849.
Oishi, H., Sasaki, T. and Takai, Y. (1996) Interaction of both the C2A and C2B domains of rabphillin3 with Ca²⁺ and phospholipid. Biochem. Biophys. Res. Commun. 229, 498-503.
Parham, R. A. and Kaustinen. H. M. (1977) On the site of tannin synthesis in plant cells. Bot. Gazette 138, 465-467.
Paris, N., Stanley, C. M., Jones, R. L. and Rogers. J. C. (1996) Plant cells contain two functionally distinct vacuolar compartments. Cell 85, 563-572.
Pavelka, M. and Robinson, D. G. (2003) The Golgi apparatus in mammalian and higher plants cells: a comparison. In, The Golgi apparatus and the plant secretory pathway. D G Robinson Ed. Sheffield, Sheffield Academic press. pp 16-35
Plant Physiol. 121, 929-38.
Reggiori, F. and Klionsky, D. L. (2002). Autophagy in the eukaryotlc cell. Eukaryotic Cell 1, 11-21.
Rojo, E., Gillmor, C. S., Kovaleva, V., Somerville, C. R. and Raikhel, N. V. VACUOLELESS1 is an essential gene required for vacuole formation and morphogenesis in Arabidopsis. Dev. Cell. 1, 303-10.
Rojo, E., Zouhar, J., Kovaleva, V., Hong, S, and Raikhel, N. V. (2003) The AtC-VPS Protein Complex is Localized to the Tonoplast and the Prevacuolar Compartment in Arabidopsis. Mol. Biol. Cell. 14, 361-9.
Sanderfoot, A. A. and Raikhel, N. V. (1999) The specificity of vesicle trafficking: coat proteins and SNAREs. Rant Cell. 11, 629-42.
Sanderfoot, A. A., Kovaleva, V., Zheng, H, and Raikhel, N. V. (1999) The t-SNARE AtVAM3p resides on the prevacuolar compartment in Arabidopsis root cells.
Sato, M. H., Nakamura, N., Ohsumi, Y., Kouchi, H., Kondo, M., Hara-Nishimura, I., Nishimura, M. and Wada, Y. (1997) The AtVAM3 encodes a syntaxin-related molecule implicated in the vacuolar assembly in Arabidopsis thaliana. J. Biol. Chem. 272, 24530-5.
Schoenbohm, C, Martens, S., Eder, C, Forkmann, G. and Weisshaar, B. (2000). Identification of the Arabidopsis thaliana flavanoid-3′-hydroxylase gene and functional expression of the encoded P450 enzyme. Biol. Chem. 381, 749-753.
Senior, I. J. (1998). Uses of plant gene silencing. Biotech. Genet. Eng. Revs. 15, 79-119.
Shirley, B. W. (1996). Flavonoid biosynthesis: “new” functions for an “old” pathway. Trends Plant Sci. 1, 377-382.
Shirley, B. W., Hanley, S. and Goodman, H. M. (1992). Effects of ionising radiation on a plant genome: analysis of two Arabidopsis transparent testa mutants. Plant Cell 4, 333-347.
Singh, S., McCallum, J., Gruber, M., Towers, G. H. N., Muir, A. D., Bonn, B. A., Koupai-Abyazani. M. R., Glass, A. D. M. (1997) Biosynthesis of flavan-3-ols by leaf extracts of Onobrychis vicifolia. Phytochem. 44, 425-432.
Smertenko, A. P., Bozhkov, P. V., Filanova, L. H., von Arnold, S. and Hussey, P. J. (2003) Reorganisation of the cytoskeleton during developmental programmed cell death in Picea ables embryos. Plant J. 33, 813-824.
Smith, N. A., Singh, S. P., Wang. M. B., Stoutjesdijk, P. A., Green, A. G. and Waterhouse, P. M. (2000). Total silencing by intron-spliced hairpin RNAs. Nature 407, 319-320.
Snyder, B. A. and Nicholson, R. L. (1990) Synthesis of phytoalexins in sorghum as a site-specific response to fungal ingress. Science 248, 1637-39.
Stafford, H. A. (1989) The enzymology of proanthocyanidin biosynthesis. In R W Hemingway and J J Karchesy, eds, Chemistry and significance of condensed tannins, Plenum Press, New York, pp 345-368.
Stafford, H. A. (1990) Flavonoid Metabolism. Florida: CRC Press.
Stahelin, R. V. and Cho, W. (2001) Roles of calcium ions in the membrane binding of C2 domains. Biochem. J. 359, 679-685.
Sutton, R. B., Davletov, B. A., Berghuls, A. M., Sudhof, T. C. and Sprang, S. R. (1995) Structure of the first C2 domain of synaptotagmin I: a novel Ca²⁺/phospholipid-binding fold. Cell. 80, 929-38.
Swanson, S. J., Bethke, P. C. and Jones, R. L. (1998) Barley aleurone cells contain two types of vacuoles: characterisation of lytic organelles by use of fluorescent probes. Plant Cell, 10, 685-698.
Tanner, G. and Kristiansen, K. N. (1993) Synthesis of 3,4-c/s-[3H]-leucocyanidin and enzymatic reduction to catechin. Anal. Biochem. 209, 274-277.
Tanner, G. J., Francki, K. T., Abrahams, S., Watson, J. M., Larkin, P. J. and Ashton, A. R., (2003). Proanthocyanidin biosynthesis in plants. Purification of legume leucoanthocyanidin reductase and molecular cloning of its cDNA. J. Biol. Chem. In press.
Tanner, G. J., Moate, P. J., Davis, L. H., Laby, R. H., Yuguang, L. and Larkin, P. A. (1995) Proanthocyanidins (condensed tannin) destabilise plant protein foams in a dose dependent manner. Aust J Agric Res 46:1101-1109.
Tanner, G. J., Moore, A. M. and Larkin, P. J. (1994). Proanthocyanidins inhibit hydrolysis of leaf proteins by rumen microflora in vitro. Br. J. Nutrition 71, 947-958.
Terai, Y., Fujii, I., Byun, S. H., Nakajima, O., Hakamatsuka, T., Ebizuka, Y. and Sankawa, U. (1996). Cloning of chalcone-flavanone isomerase cDNA from Pueraria lobata and its overexpression in Escherichia coli. Prot. Express. Purif. 8, 183-190.
Terril T. H., et al. (1992) The effect of condensed tannins upon body growth, wool growth and rumen metabolism in sheep grazing Sulla (Hedysarum coronarium) and perennial pasture. J. Agric. Sci. 119, 265-273.
Thompson, J. D., et al. (1994) Nucl. Acids Res. 22, 4673-4680.
Ubach, J., Garcia, J., Nittler, M. P., Sudhof, T. C. and Rizo, J. (1999) Structure of the Janus-faced C2B domain of rabphillin. Nature Cell Biol. 1, 106-12.
Velten, J. and Schell, J. (1985) Selection expression plasmid vectors for use in genetic transformation of higher plants Nucl. Acids Res. 13, 6981-6999.
Verdaguer, N., Corbalan-Garcia, S., Ochoa W. F., Fita I. and Gomez-Fernandez, J. C. (1999) Ca²⁺ bridges the C2 membrane-binding domain of protein kinase C directly to phosphatidyl serine. EMBO J. 18, 6329-6338.
Vitale. A. and Raikhel, N. V. (1999). What do proteins need to reach different vacuoles Trends Plant Sci. 4, 149-155.
Voisey, C. R., et al. (1994) Agrobacterium-mediated transformation of white clover using direct shoot organogenesis. Plant Cell Rep 13, 309-314.
Wagner, G. J. and Hrazdina, G. (1984) Endoplasmic reticulum as a site of phenylpropanoid and flavonol metabolism in Hippeastrum. Plant Phys. 74, 901-906.
Wang, M. B., Li, Z., Matthews, P. R., Upadhyaya, N. M. and Waterhouse, P. M. Improved vectors for Agrobacterium tumefaciens mediated transformation of monocot plants ISHS ACTA Horticulture 461: international symposium on biotechnology of tropical and subtropical species part2.
Wang Y., et al. (1994) The effects of condensed tannin in Lotus corniculatus upon nutrient metabolism and upon body and wool growth in grazing sheep. Proc. 1994 NZAPS Conference.
Weigel, D., Ahn, J. H., Blázquez, M. A. Borevitz, J. O., Christensen, S. K., Fankhauser, C, Ferrandiz, C, Kardailsky, I., Malancharuvil, E. J., Neff, M. M., Nguyen, J. T., Sato, S., Wang, Z. Y., Xia, Y., Dixon, R. A., Harrison, M. J., Lamb, C. J., Yanofsky. M. F. and Chory, J. (2000). Activation Tagging in Arabidopsis. Plant Physiol. 122, 1003-1014.
White, D. L., Andrews, S. B., Faller, J. W. and Barrnett, R. J. (1976) The chemical nature of osmium tetroxide fixation and staining of membranes by x-ray photoelectron spectroscopy. Biochim. Biophys. Acta 436, 577-592.
Winkel-Shirley, B. (2001) Flavonoid biosynthesis. A colourful model for genetics, biochemistry, cell biology and biotechnology. Plant Physiol. 126, 485-493.
Xie, D. Y, Sharma. S. B., Palva, N. L., Ferreira, D. and Dixon, R. A. (2003). Role of anthocyanidin reductase, encoded by BANYULS in plant flavonoid biosynthesis. Science 222, 396-399.
Young, H, Paterson, V. J. (1980) Condensed tannins from white clover seed diffusate. Phytochemistry 19, 159-160.

1.-48. (canceled)

49. A method for identifying a nucleic acid molecule which increases proanthocyanidin levels in a plant tissue or organ comprising:
a) transforming a plant cell with a nucleic acid molecule comprising a nucleic acid sequence that encodes an amino acid sequence which is at least 40% identical overall to SEQ ID NO:18 or a complement of the nucleic acid sequence;
b) producing a plant tissue or organ from the transformed plant cell of step a); and
c) selecting a tissue or organ of step b) having an increased proanthocyanidin level,
wherein an increase level of proanthocyanidin in the transformed tissue or organ relative to the level of proanthocyanidin in a corresponding tissue or organ not transformed with the nucleic acid molecule identifies the nucleic acid molecule which increases proanthocyanidin levels in a plant tissue or organ.

50. The method of claim 49, comprising a step of producing a transformed plant from the transformed plant cell of step a).

51. The method of claim 49, comprising a step of producing a progeny plant from the transformed plant.

52. The method of claim 49, wherein the tissue or organ of the plant is a leaf or stem.

53. The method of claim 49, wherein the tissue or organ of the plant is a seed.

54. A method for producing a plant with increased levels of proanthocyanidin, comprising:
a) transforming a plant cell with a nucleic acid molecule comprising a nucleic acid sequence that encodes an amino acid sequence which is at least 40% identical overall to SEQ ID NO:18 or a complement of the nucleic acid sequence;
b) producing a plant tissue or organ from the transformed plant cell of step a); and
c) selecting a tissue or organ of step b) having an increased proanthocyanidin level,
wherein the selected plant tissue or organ having an increased level of proanthocyanidin in the transformed tissue or organ relative to the level of proanthocyanidin in a corresponding tissue or organ not transformed with the nucleic acid molecule is used to produce the plant, or a progeny of the plant, with increased proanthocyanidin levels.


SEQUENCE LISTING (45 sequences)

SEQ ID NO: 1 - DNA, 1039 bp, Arabidopsis
aagctttggtagcaaaaaaattgaattcataaaaagaatacgtcgtcttttagatcacaagctttgaacgttagcaaaattatgttacctaccaccagtccctggcttattatatagagagagatattcacatttacaatttcgatattgtttctctaagatcgtaaaatagtacaccaagccatttataaatttcttcactctcagattcaaaataatataaattagaaatgggaacagagatggtcatggttcacgaggttccttttcctccacagatcatcacttccaagccactctctcttctgggccaagggatcacagacattgagatccactttcttcaagtgaagttcactgcgatcggagtttacttagatccttcagatgttaaaacacatcttgataactggaaaggcaaaaccggaaaagaactcgccggcgatgatgacttcttcgacgcccttgcctccgcggagatggagaaggttataagagtggtggtgataaaggagataaaaggagctcagtacggagtgcagctagagaatacggtgagagatcgtttggctgaggaggataagtacgaggaagaagaagaaactgagctcgagaaggtcgttggctttttccagtccaagtacttcaaagctaactccgttatcacttaccatttctcagccaaagatggcatttgcgagatcgggtttgagacggaaggtaaagaagaggagaaactgaaggtggagaatgcgaatgtggtgggaatgatgcagagatggtatttgtccggaagtcgtggagtctcaccgtcgactattgtctccatcgctgattcaatctccgcagttttaacctaagaaccatggccccttacataaaaatttgtatgtgtgtgtgttttatgtagtatttttttttttcctcttgtcaagttcctgcttttgtatcatcaaacttttactttggagtttgatatggccgaacaatatggtaattccttaatctcaatttaaatcagctttgctcgtttggattc

SEQ ID NO: 2 - protein, 209 aa, Arabidopsis
MGTEMVMVHEVPFPPQIITSKPLSLLGQGITDIEIHFLQVKFTAIGVYLDPSDVKTHLDNFKGKTGKELAGDDDFFDALASAEMEKVIRVVVIKEIKGAQYGVQLENTVRDRLAEEDKYEEEEETELEKVVGFFQSKYFKANSVITYHFSAKDGICEIGFETEGKEEEKLKVENANVVGMMQRFYLSGSRGVSPSTIVSIADSISAVLT

SEQ ID NO: 3 - DNA, 898 bp, Arabidopsis
agaaagtgagtaaggaagaaataatcaggaacattaataaaggaaatgacaacggcgtgtccggcgaggacaagctcactaatggacgatctcttgggcctccttagaatccgtatcaaacgtggcgtcaatttagccgtccgtgacatcagtagcagcgatccttacgtcgtcgtcaagatgggcaaacagaaattgaagactcgtgtcattaacaaagatgttaatccagaatggaatgaagatttgactctctccgtcacagactccaatcttaccgtcctcttgacggtgtatgatcacgacatgtttagcaaagacgacaagatgggcgatgctgagttcgagataaagccctatattgaggctttgaggatgcaactcgatggacttcctagtggaaccattgtgaccacggttaagccaagtcgtcgtaactgtctagccgaggagtcacgtgtcacctgggtcgacggcaagctcgtccaggatctcgttctcagattgcgacatgtggaatgcggagaggtcgaggctcagcttcaatggattgacctccctggctccaagggtctatgaatcttttttcttttcttatttaccgttactttgagttgttttctttcaaattgaactaaaccaagtaacagcatggaaaaaaacaagtaatttcaatcttgaaggatatagtgaactagaaacacgtattaatccatggaaatataaggattaagaacgttgagggaagttttgttcccatatggtgatcgagtccaagtcgagtcgtggtggtggatatgtaaatgtggtgtattgttgatgtgtatgtatatttgggctagctatgccttaaataaggcatttacacgaacaactaatacagatattttatttattt

SEQ ID NO: 4 - protein, 177 aa, Arabidopsis
MTTACPARTSSLMDDLLGLLRIRIKRGVNLAVRDISSSDPYVVVKMGKQKLKTRVINKDVNPEFNEDLTLSVTDSNLTVLLTVYDHDMFSKDDKMGDAEFEIKPYIEALRMQLDGLPSGTIVTTVKPSRRNCLAEESRVTFVDGKLVQDLVLRLRHVECGEVEAQLQFIDLPGSKGL

SEQ ID NO: 5 - DNA, 630 bp, Arabidopsis
atggcgagtgaaagggttttggttgatgaaatctcttatccttccaagatcactactaccaagcctctttctctccttggtcatgggattaccgacatggagatccattttctgcaagtgaaattttattcaattggggtttatttggaacctgaagtagtaagccacttgcaacaatggaaaggtaaacctgccaaggagttggaaggcagtgatgacttctttgatgctctcatttctgctcctgtggagaaggtaatcagacttgtggtgattaaagagattaagggtgcacagtatggtcttcaagttgaaactgctgtgagggaccgtttggcagctgatgacaaatacgaggatgaagaagaagaagctttggaaaaagtagttgaatttttccggtccaagtacttcaagaaaaactcagtcatcacatatcatttccctgcaaattctacaactgctgagattgcggtatctttggagggaaaagacgacacaaagtatgtaatagagaatgccaatgtggtagagacaatccagaaatggtaccttgctggctcaagggctatatctccttcaaccatttcatctttggctgcaaatctctcagaggaattgtccaagtga

SEQ ID NO: 6 - protein, 233 aa, Oryza sativa
MAAVSEVEVDGVVFPPVARPPGSGHAHFLAGAGVRGVEIAGNFIKFTAIGVYLEEGAAVPALAKKWAGKSADELAADAAFFRDVVTGDFEKFTRVTMILPLTGEQYSDKVTENCVAAWKAAGVYTDAEGAAADKLKEAFKPHSFPPGASILFTHSPAGVLTVAFSKDSSVPEGAVAAAAIENRALCEAVLDSIIGEHGVSPAAKRSIAARVSQLLKAESTGDVAAAEPAPVSA

SEQ ID NO: 7 - protein, 246 aa, Hordeum vulgare
MAVSELEVDGVVFPPLARPPGSAHAHFLAGAGVRGMEIGGHFIKFTAIGVYLQADAAVSALAAKWAGKPAADLASDAAFFRDVVTGEFEKFTRVTMILPLTGAQYSDKVTMILPLTGAQYSDKVTENCVAYWKAAGVYTDAEAAAVDKFKEAFGPHSFAPGASILFTHSPAGVLTVAFSKDSSVPESGGVAIENARLCEAVLESIIGEHGVSPAAKLSLANRVAELLKGAAHAGGEPAAEPVPVSV

SEQ ID NO: 8 - protein, 231 aa, Zea mays
MACRRWWSTAVVFPPVARPPGSAGSHFLGGAGVRGVEIGGNFIKFTAIGVYLEDAAVPALAKKWGGKTADELASDAAFFRDVVTGDFEKFTRVTMILPLTGEQYAEKVTENCVAFWKAAGLYTDAEGVAVEKFREVFKPETFAPGRSILFTHSPAGVLTVAFSKDSSVPAAGGVAIENKRLCEAVLESIIGERGVSPAAKLSLAARVSELLAKETAAAADAPQAEPVSITA

SEQ ID NO: 9 - protein, 248 aa, Arabidopsis thaliana
MSSSNACASPSPFPAVTKLHVDSVTFVPSVKSPASSNPLFLGGAGVRGLDIQGKFVIFTVIGVYLEGNAVPSLSVKWKGKTTEELTESIPFFREIVTGAFEKFIKVTMKLPLTGQQYSEKVTENCVAIWKQLGLYTDCEAKAVEKFLEIFKEETFPPGSSILFALSPTGSLTVAFSKDDSIPETGIAVIENKLLAEAVLESIIGKNGVSPGTRLSVAERLSQLMMKNKDEKEVSDHSVEEKLAKENRE

SEQ ID NO: 10 - protein, 222 aa, Citrus sinensis
MNPSPSVTELQVENVTFTPSVQPPGSTKSHFLGGAGERGLEIEGKFVKFTAIGVYLEDDAVPLLAGKWKGKTAEELTESVEFFRDVVTGPFEKFMKVTMILPLTGAQYSEKVAENCIAIWKFFGIYTDAEAKAIEKFTEVFKDEIFPPGSSILFTQSPGSLTISFSKDGSIPKDGVABIESNLLSEAVLESMIGKNGVSPAAKKSLAERLSALLNVTSDKMK

SEQ ID NO: 11 - protein, 234 aa, Vitus vinifera
MSQVPSVTAVQVENVLFPPSVKPPGSTNDLFLGGAGVRGLEIQGKFVKFTAIGVYLENSAVPTLAVKWKGKTVEELADSVDFFRDVVTGPFEKFTKVTTILPLTGRQYSDKVSENCVAFWKSVGIYTDAEAKAIEKFNEVLKDETFPPGNSILFTHSPLGALTMSFSKDGSLPEVGNAVIENKLLTEAVLESIIGKHGVSPEAKKSLAARLSELFCKEAGDEKIEAEKVAPVAC

SEQ ID NO: 12 - protein, 256 aa, Elaegnus umbellate
MAPFTKSVTEVQVESVIFPPEVKPPGSSKTLFLGGAGVRGIEIQGKFIKFTAIGVYLEDNAVPSLAVKWKGKSAQELTESVEFFRDIVTGPMEKFTRVTTILPLTGQQYSEKVSENCVAAWKSLGIYSDAEAKAIEKFIEIFKDQTFPPAASNLFTQSPLGSLTMSFSKDGSIPEVGNAVLENKLLSEABLESIIGKHGVSPEAKQNLATRLVQLLNENSTTDLNESENEKLNSNEVSKEEKPLQVEKSAFKEVEV

SEQ ID NO: 13 - protein, 233 aa, Saussurea medusa
MAPPPSSTSIQVESIVFPPSVKPPGATTTLFLGGAGVRGMEIQGNFVKFTGIGVYLEDKAIPSLAVKWKGKTAAELTDSVQRYRDIVTGPFEKFSQVTMILPLTGKQYSEKVSEMCIGVWKAQGTYTDADTATIEKFLEVFKDENFLPGSSILFTTSPTGSLTISFSKDGNIPEAATVVLENRKLAQTVIESVIGEHGVSPEAKQSLASRLSDFMTQFDEAKATANVESQIGL

SEQ ID NO: 14 - protein, 240 aa, Petunia hybrida
MSPPVSVTKMQVENYAFAPTVNPAGSTNTLFLAGAGHRGLEIEGKFVKFTAIGVYLEESAIPFLAEKWKGKTPQELTDSVEFFRDVVTGPFEKFTRVTMILPLTGKQYSEKVAENCVAHWKGIGTYTDDEGRAIEKFLDVFRSETFPPGASIMFTQSPLGLLTISFAKDSVTGTANAVIENKQLSEAVLESIIGKHGVSPAAKCSVAERVAELLKKSYAEEASVFGKPETEKSTIPVIGV

SEQ ID NO: 15 - protein, 218 aa, Glycine max
MATISAVQVEFLEFPAVVTSPASGKTYFLGGAGERGLTIEGKFIKFTGIGVYLEDKAVPSLAAKWKGKTSEELVHTLHFYRDIISGPFEKLIRGSKILPLAGAEYSKKVMENCVAHMKSVGTYGDAEAAAIEKFAEAFKNVNFAPGASVFYRQSPDGILGLSFSEDATIPEKEAAVIENKAVSAAVLETMIGEHAVSPDLKRSLASRLPAVLSHGIIV

SEQ ID NO: 16 - protein, 221 aa, Phaseolus vulgaris
MATAPTITDVQVEFLHFPAVVTSPATAKTYFLGGAGERGLTIEGKFIKFTAIGVYLEDKAVASLATKWKGKPSEELINTLDFYRDIISGPFEKLIRGSKILQLSGTEYSRKVMENCVAHLKSVGTYGDAEAKGIEEFAEAFKKVNFPPGASVFYRQSPDGILGLSFSEDATIPGEEAVVIENKAVSAAVLETMIGEHAVSPDLKRSLASRLPAVLNGGIIV

SEQ ID NO: 17 - protein, 223 aa, Medicago sativa
MAASITAITVENLEYPAVVTSPVTGKSYFLGGAGERGLTIEGNFIKFTAIGVYLEDIAVASLAAKWKGKSSEELLETLDFYRDIISGPFEKLIRGSKIRELSGPEYSRKVMENCVAHLKSVGTYGDAEAEAMQKFAEAFKPVNFPPGASVFYRQSPDGILGLSFSPDTSIPEKEAALIENKAVSSAVLETMIGEAHAVSPDLKRCLAARLPALLNEGAFKIGN

SEQ ID NO: 18 - protein, 209 aa, A.thaliana
MGTEMVMVHEVPFPPQIITSKPLSLLGQGITDIEIHFLQVKFTAIGVYLDPSDVKTHLDNWKGKTGKELAGDDDFFDALASAEMEKVIRVVVIKEIKGAQYSVQLENTVRDRLAEEDKYEEEEETELEKVVGFFQSKYFKANSVITYHFSAKDGICEIGFETEGKEEEKLKVENANVVGMMQRWYLSGSRGVSPSTIVSIADSISAVLT

SEQ ID NO: 19 - protein, 31 aa, Arabidopsis
  MISC_FEATURE (1)..(1): X=KN
XXLXLXXXGXTDXEIHFXXXKXXXXBVYXXX

SEQ ID NO: 20 - protein, 17 aa, Arabidopsis
  MISC_FEATURE (1)..(1): X=VI
XVVXKEXKGXQYGVQLE

SEQ ID NO: 21 - protein, 32 aa, Arabidopsis
  MISC_FEATURE (1)..(1): X=VI
XRDRXXXXDXXXXXEEXXLEKXXXFFQXKYFX

SEQ ID NO: 22 - protein, 6 aa, Arabidopsis
ENANVV

SEQ ID NO: 23 - protein, 31 aa, Arabidopsis
KPLSLLGQGITDIEIHFLQVKFTAIGVYLDP

SEQ ID NO: 24 - protein, 18 aa, Arabidopsis
RVVVIKEIKGAQYGVQLE

SEQ ID NO: 25 - protein, 32 aa, Arabidopsis
VRDRLAEEDKYEEEEETELEKVVGFFQSKYFK

SEQ ID NO: 26 - protein, 6 aa, Arabidopsis
ENANVV

SEQ ID NO: 27 - DNA, 29 bp, Arabidopsis
ccagtccaagtacttcaaagctaactccg

SEQ ID NO: 28 - DNA, 35 bp, Arabidopsis
tcccccgggatggttcttaggttaaaactgcggag

SEQ ID NO: 29 - DNA, 26 bp, Arabidopsis
cgtcacagactccaatcttaccgtcc

SEQ ID NO: 30 - DNA, 32 bp, Arabidopsis
ccgagctcccatgctgttacttggtttagttc

SEQ ID NO: 31 - DNA, 25 bp, Arabidopsis
cgttcctctactggtactcgggtcc

SEQ ID NO: 32 - DNA, 24 bp, Arabidopsis
cacaagcacgatgacacagagaac

SEQ ID NO: 33 - DNA, 34 bp, Arabidopsis
ggaattcgtgagtaaggaagaaataatcaggaac

SEQ ID NO: 34 - DNA, 33 bp, Arabidopsis
cccaagctttagacccttggagccagggaggtc

SEQ ID NO: 35 - DNA, 31 bp, Arabidopsis
cgggatccagagatggtcatggttcacgagg

SEQ ID NO: 36 - DNA, 29 bp, Arabidopsis
cccaagcttggttaaaactgcggagattg

SEQ ID NO: 37 - DNA, 32 bp, Arabidopsis
cgggatcccagagatggtcatggttcacgagg

SEQ ID NO: 38 - DNA, 35 bp, Arabidopsis; identical to SEQ ID NO: 28

SEQ ID NO: 39 - DNA, 34 bp, Arabidopsis
  misc_feature (12)..(12): N=I
ggatccatggcnastgaaanngttttggttgatg

SEQ ID NO: 40 - DNA, 31 bp, Arabidopsis
cggatccttcacttggacaaytcctsygaga

SEQ ID NO: 41 - DNA, 35 bp, Arabidopsis
gctctagagtgagtaaggaagaaataatcaggaac

SEQ ID NO: 42 - DNA, 32 bp, Arabidopsis; identical to SEQ ID NO: 30

SEQ ID NO: 43 - protein, 85 aa, Arabidopsis
LLRIRIKRGVNLAVRDISSSDPYVVVKMGKQKLKTRVINKDVNPEWNEDLTLSVTDSNLTVLLTVYDHDMFSKDDKMGDAEFEIK

SEQ ID NO: 44 - protein, 90 aa, Arabidopsis
TLTVKIISARNLPPKDKGGKSDPYVKVSLDGDPREKKKTKVVKNTLNPVWNETFEFEVPPPELSELEIEVYDKDRFSRDDFIGRVTIPLS

SEQ ID NO: 45 - DNA, 4 bp, Arabidopsis
tata